(19) United States
(12) Patent Application Publication
(10) Pub. No.: US 2022/0347292 A1
(43) Pub. Date: Nov. 3, 2022

(54) HUMAN CYTOMEGALOVIRUS VACCINE

(71) Applicant: ModernaTX, Inc., Cambridge, MA (US)

(72) Inventors: Lori Panther, Cambridge, MA (US); Shinu John, Cambridge, MA (US); Tal Zaks, Cambridge, MA (US)

(73) Assignee: ModernaTX, Inc., Cambridge, MA (US)

(21) Appl. No.: 17/641,967

(22) PCT Filed: Sep. 11, 2020
(86) PCT No.: PCT/US2020/050392

§ 371 Date: Mar. 10, 2022

Related U.S. Application Data
(60) Provisional application No. 62/958,623, filed on Jan. 8, 2020.
(60) Provisional application No. 62/899,624, filed on Sep. 12, 2019.
(60) Provisional application No. 62/899,129, filed on Sep. 11, 2019.

(51) Int. Cl. A61K 39/245; A61P 31/22

(52) U.S. Cl. CPC A61K 39/245; A61P 31/22; A61K 2039/545

(57) ABSTRACT

Aspects of the invention relate to methods for producing an antigen-specific immune response to human cytomegalovirus (hCMV) in a subject by administering mRNA vaccines comprising hCMV antigenic polypeptides gH, gL, UL128, UL130, UL131 A and gB formulated in lipid nanoparticles, wherein the antigen-specific immune response to hCMV results in neutralizing antibodies that have i) a geometric mean titer of at least 3-fold against epithelial cell infection or ii) a geometric mean ratio of 9-41 against epithelial cell infection or iii) a geometric mean ratio of 4-8-fold against fibroblast infection.

### CROSS REFERENCE TO RELATED APPLICATIONS

This application claims the benefit under 35 U.S.C. § 119(e) of U.S. Provisional Application No. 62/899,129, filed Sep. 11, 2019, entitled “Human Cytomegalovirus Vaccine,” U.S. Provisional Application No. 62/899,624, filed Sep. 12, 2019, entitled “Human Cytomegalovirus Vaccine,” and U.S. Provisional Application No. 62/958,623, filed Jan. 8, 2020, entitled “Human Cytomegalovirus Vaccine,” the entire disclosure of each of which is hereby incorporated by reference in its entirety.

### REFERENCE TO A SEQUENCE LISTING SUBMITTED AS A TEXT FILE VIA EFSWEB

The instant application contains a Sequence Listing which has been submitted in ASCII format via EFS-Web and is hereby incorporated by reference in its entirety. The ASCII file, created on Sep. 4, 2020, is named M137870131WO00-SEQ-OMJ.txt and is 55 kilobytes in size.

### BACKGROUND

Cytomegalovirus (CMV) is a member of the Herpesviridae family of viruses. CMV is primarily acquired through contact with infectious mucosal secretions or in utero, and establishes latency after primary infection. Overall, CMV seroprevalence in the United States is 50.4%, but rates of 60% to 100% have been reported in resource-poor areas.

CMV is the most common congenital viral infection, as it affects 30,000 to 40,000 infants in the United States annually (0.6% to 2% of live births). Although congenital CMV infection in the first trimester is associated with the most adverse pregnancy outcomes, symptomatic congenital CMV can result from infection at any time during pregnancy. Approximately 30% to 35% of mothers with primary CMV infection during pregnancy will transmit the virus to the fetus; 12% of these newborns will have symptomatic disease, and approximately 4% will die in the first year of life. In addition, approximately half of CMV-infected infants who are symptomatic at birth will develop late complications such as intellectual disability, sensorineural hearing loss, and developmental delay. Due to the significant effect that congenital CMV infection has on pediatric health, a 2017 Institute of Medicine Report places development of a CMV vaccine for the prevention of congenital CMV infection in its highest priority category.

In individuals on chronic immunosuppressive medications after solid organ or hematopoietic stem cell transplantation, CMV infection that leads to graft rejection or end-organ disease is associated with high mortality. In the United States, approximately 30,000 adults receive solid organ transplants and 22,000 receive hematopoietic cell transplants annually. Overall, 8% to 40% of solid organ transplants and 3% to 6% of hematopoietic cell transplant patients who receive antiviral prophylaxis will develop post-transplant complications due to CMV. Major complications of CMV infection in transplant recipients include acute or chronic rejection of the transplanted tissue and invasive diseases such as colitis, hepatitis, and encephalitis.

### SUMMARY

A significant unmet medical need is a safe and effective method for the prevention of congenital CMV infection. Another unmet medical need is the prevention of CMV infection in individuals on chronic immunosuppressive medications after solid organ or hematopoietic stem cell transplantation.

A messenger ribonucleic acid (mRNA)-based vaccine platform has been developed based on the principle and observations that target viral antigens can be produced in vivo by delivery and cellular uptake of the corresponding mRNA. The mRNA then undergoes intracellular ribosomal translation to endogenously express the protein antigens encoded by the vaccine mRNA. These mRNA-based vaccines do not enter the cellular nucleus or interact with the human genome, are nonreplicating, and are expressed transiently. mRNA vaccines thereby offer a mechanism to stimulate the endogenous production of structurally intact protein antigens in a manner that mimics wild-type viral infection and is able to induce highly targeted immune responses against infectious pathogens such as CMV.

Described herein, in some aspects, is a messenger ribonucleic acid (mRNA)-based prophylactic vaccine (designated herein as hCMV mRNA vaccine A) comprising mRNA encoding full length CMV glycoprotein B (gB) and mRNA encoding the pentameric gH/gL/UL128/UL130/UL131A glycoprotein complex.

Some aspects of the present disclosure provide methods for producing an antigen-specific immune response to human cytomegalovirus (hCMV) in a human subject comprising administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle, thereby inducing an antigen-specific immune response to hCMV in the subject, wherein the geometric mean titer (GMT) of neutralizing antibodies against epithelial cell infection increases in the human subject at least 3-fold relative to baseline following administration of the immunogenic composition.

Further aspects of the present disclosure provide methods for producing an antigen-specific immune response to human cytomegalovirus (hCMV) in a human subject comprising administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle, thereby inducing an antigen-specific immune response to hCMV in the subject, wherein the geometric mean ratio (GMR) of neutralizing antibodies against epithelial cell infection in the human subject is about 9-41 following administration of the immunogenic composition.

Further aspects of the present disclosure provide methods for producing an antigen-specific immune response to human cytomegalovirus (hCMV) in a human subject comprising administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle, thereby inducing an antigen-specific immune response to hCMV in the subject, wherein the geometric mean ratio (GMR) of neutralizing antibodies against fibroblast infection in the human subject is about 4-8 following administration of the immunogenic composition.

Further aspects of the present disclosure provide compositions for use in producing an antigen-specific immune response to human cytomegalovirus (hCMV) in a human subject wherein the use comprises administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle, thereby inducing an antigen-specific immune response to hCMV in the subject, wherein the geometric mean titer (GMT) of neutralizing antibodies against epithelial cell infection increases in the human subject at least 3-fold relative to baseline following administration of the immunogenic composition.

Further aspects of the present disclosure provide compositions for use in producing an antigen-specific immune response to human cytomegalovirus (hCMV) in a human subject wherein the use comprises administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle, thereby inducing an antigen-specific immune response to hCMV in the subject, wherein the geometric mean ratio (GMR) of neutralizing antibodies against epithelial cell infection in the human subject is about 9-41 following administration of the immunogenic composition.

Further aspects of the present disclosure provide compositions for use in producing an antigen-specific immune response to human cytomegalovirus (hCMV) in a human subject wherein the use comprises administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle, thereby inducing an antigen-specific immune response to hCMV in the subject, wherein the geometric mean ratio (GMR) of neutralizing antibodies against fibroblast infection in the human subject is about 4-8 following administration of the immunogenic composition.

In some embodiments, the immunogenic composition is administered at a dose of 30 μg. In some embodiments, the immunogenic composition is administered at a dose of 90 μg. In some embodiments, the immunogenic composition is administered at a dose of 180 μg. In some embodiments, the immunogenic composition is administered at a dose of 300 μg.

In some embodiments, at least two doses or at least three doses of the immunogenic composition are administered. In some embodiments, three doses of the immunogenic composition are administered. In some embodiments, doses of the immunogenic composition are administered on: Day 1; around the beginning of month 2; and around the beginning of month 6. In some embodiments, administration of a single dose of the immunogenic composition elicits serum neutralizing antibody titers against hCMV.

In some embodiments, the GMT of neutralizing antibodies against epithelial cell infection increases in the human subject at least 3-fold relative to baseline following a single dose, following two doses, or following three doses of the immunogenic composition. In some embodiments, the GMT of neutralizing antibodies against epithelial cell infection increases in the human subject at least 3-fold relative to baseline following two doses or following three doses of the immunogenic composition. In some embodiments, the GMT of neutralizing antibodies against epithelial cell infection increases in the human subject 9-20 fold relative to baseline following two doses of the vaccine composition. In some embodiments, the GMT of neutralizing antibodies against epithelial cell infection increases in the subject 20-40-fold relative to baseline following three doses of the vaccine composition.

In some embodiments, the GMR of neutralizing antibodies against epithelial cell infection in seropositive subjects administered at least 2 doses of ≥30 μg of the immunogenic composition is in the range of 14-26. In some embodiments, the GMR of neutralizing antibodies against epithelial cell infection in seropositive subjects administered at least 3 doses of ≥30 μg of the immunogenic composition is in the range of 14-26. In some embodiments, the GMR of neutralizing antibodies against epithelial cell infection in seropositive subjects administered at least 3 doses of ≥30 μg of the immunogenic composition is in the range of 14-41. In some embodiments, the GMR is in the range of 30-41. In some embodiments, at least 3 doses of about 180 μg are administered to the seropositive subjects.

In some embodiments, the lipid nanoparticle comprises: an ionizable cationic lipid; cholesterol; 1,2-distearoyl-sn-glycero-3-phosphocholine (DSPC); and 1,2 dimyristoyl-sn-glycerol, methoxypolyethyleneglycol (DMG-PEG). In some embodiments, the ionizable cationic lipid comprises Compound I:

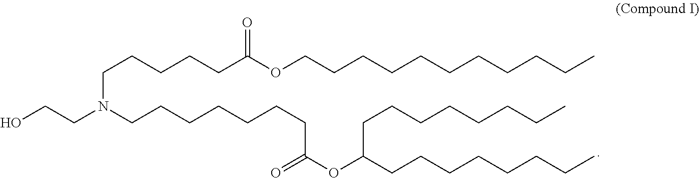 [SMILES: CCCCCCCCCCCOC(=O)CCCCCN(CCO)CCCCCCCC(=O)OC(CCCCCCCC)CCCCCCCC]

In some embodiments, the lipid nanoparticle comprises a mixture of lipids comprising 20-60 mol % ionizable cationic lipid, 25-55 mol % cholesterol, 5-25 mol % DSPC, and 0.5-15 mol % DMG-PEG. In some embodiments, the lipid nanoparticle comprises a mixture of lipids comprising 45-55 mol % ionizable cationic lipid, 35-40 mol % cholesterol, 5-15 mol % DSPC, and 1-2 mol % DMG-PEG. In some embodiments, the lipid nanoparticle comprises a mixture of lipids comprising 50 mol % ionizable cationic lipid, 38.5 mol % cholesterol, 10 mol % DSPC, and 1.5 mol % DMG-PEG.

In some embodiments, the weight ratio of the mRNA encoding hCMV gH, gL, UL128, UL130, UL131A, and gB proteins in the vaccine composition is 1:1:1:1:1:1. In some embodiments, the mRNA encoding hCMV gH, gL, UL128, UL130, UL131A, and gB proteins comprise a 1-methylpseudourine chemical modification.

In some embodiments, the mRNA encoding hCMV gH protein comprises a nucleotide sequence having at least 90% identity to the sequence of SEQ ID NO: 5. In some embodiments, the mRNA encoding hCMV gH protein comprises the nucleotide sequence of sequence of SEQ ID NO: 5.

In some embodiments, the mRNA encoding hCMV gL protein comprises a nucleotide sequence having at least 90% identity to the sequence of SEQ ID NO: 6. In some embodiments, the mRNA encoding hCMV gL protein comprises the nucleotide sequence of sequence of SEQ ID NO: 6.

In some embodiments, the mRNA encoding hCMV UL128 protein comprises a nucleotide sequence having at least 90% identity to the sequence of SEQ ID NO: 2. In some embodiments, the mRNA encoding hCMV UL128 protein comprises the nucleotide sequence of sequence of SEQ ID NO: 2.

In some embodiments, the mRNA encoding hCMV UL130 protein comprises a nucleotide sequence having at least 90% identity to the sequence of SEQ ID NO: 3. In some embodiments, the mRNA encoding hCMV UL130 protein comprises the nucleotide sequence of sequence of SEQ ID NO: 3.

In some embodiments, the mRNA encoding hCMV UL131A protein comprises a nucleotide sequence having at least 90% identity to the sequence of SEQ ID NO: 4. In some embodiments, the mRNA encoding hCMV UL131A protein comprises the nucleotide sequence of sequence of SEQ ID NO: 4.

In some embodiments, the mRNA encoding hCMV gB protein comprises a nucleotide sequence having at least 90% identity to the sequence of SEQ ID NO: 1. In some embodiments, the mRNA encoding hCMV gB protein comprises the nucleotide sequence of sequence of SEQ ID NO: 1.

In some embodiments, the mRNA encoding hCMV gH protein comprises the nucleotide sequence of sequence of SEQ ID NO: 5, the mRNA encoding hCMV gL protein comprises the nucleotide sequence of sequence of SEQ ID NO: 6, the mRNA encoding hCMV UL128 protein comprises the nucleotide sequence of sequence of SEQ ID NO: 2, the mRNA encoding hCMV UL130 protein comprises the nucleotide sequence of sequence of SEQ ID NO: 3, the mRNA encoding hCMV UL131A protein comprises the nucleotide sequence of sequence of SEQ ID NO: 4, and the mRNA encoding hCMV gB protein comprises the nucleotide sequence of sequence of SEQ ID NO: 1.

In some embodiments, the open reading frame encoding the hCMV gH polypeptide comprises a sequence having at least 90% identity to the sequence of SEQ ID NO: 11, the open reading frame encoding the hCMV gL polypeptide comprises a sequence having at least 90% identity to the sequence of SEQ ID NO: 12, the open reading frame encoding the hCMV UL128 polypeptide comprises a sequence having at least 90% identity to the sequence of SEQ ID NO: 8, the open reading frame encoding the hCMV UL130 polypeptide comprises a sequence having at least 90% identity to the sequence of SEQ ID NO: 9, the open reading frame encoding the hCMV UL131A polypeptide comprises a sequence having at least 90% identity to the of sequence of SEQ ID NO: 10, and/or the open reading frame encoding the hCMV gB polypeptide comprises a sequence having at least 90% identity to the sequence of SEQ ID NO: 7.

In some embodiments, the immunogenic composition is administered via intramuscular injection.

In some embodiments the human subject is CMV-seropositive prior to being administered the hCMV mRNA immunogenic composition. In some embodiments, the human subject is CMV-seronegative prior to being administered the hCMV mRNA immunogenic composition.

In some embodiments, the methods provided further comprise administering to a human subject a dose of 5 μg to 100 μg of a second immunogenic composition comprising at least one messenger ribonucleic acid (mRNA) polynucleotide comprising an open reading frame encoding a hCMV pp65 polypeptide, wherein the mRNA polynucleotide is formulated in at least one lipid nanoparticle. In some embodiments, the second immunogenic composition is administered at a dose of 10 μg. In some embodiments, the second immunogenic composition is administered at a dose of 40 μg. In some embodiments, the second immunogenic composition is administered at a dose of 80 μg.

In some embodiments, the mRNA encoding hCMV pp65 protein comprises a nucleotide sequence having at least 90% identity to the sequence of SEQ ID NO: 21. In some embodiments, the mRNA encoding hCMV pp65 protein comprises the nucleotide sequence of sequence of SEQ ID NO: 21.

In some embodiments, the open reading frame encoding the hCMV pp65 polypeptide comprises a sequence having at least 90% identity to the sequence of SEQ ID NO: 23. In some embodiments, the second vaccine composition is administered via intramuscular injection.

In some embodiments, the open reading frame encoding the hCMV gH polypeptide comprises SEQ ID NO: 11, the open reading frame encoding the hCMV gL polypeptide comprises SEQ ID NO: 12, the open reading frame encoding the hCMV UL128 polypeptide comprises SEQ ID NO: 8, the open reading frame encoding the hCMV UL130 polypeptide comprises SEQ ID NO: 9, the open reading frame encoding the hCMV UL131A polypeptide comprises SEQ ID NO: 10, and/or the open reading frame encoding the hCMV gB polypeptide comprises the sequence of SEQ ID NO: 7. In some embodiments, the open reading frame encoding the hCMV pp65 polypeptide comprises SEQ ID NO: 23.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 depicts an overview of the study design. Abbreviations: IST, Internal Safety Team; SMC, Safety Monitoring Committee.

FIG. 2 shows blood sampling time points in dose-escalation phase A. Abbreviations: A, blood sample for antibody-mediated immunogenicity; S, safety blood sample. “Visit” denotes clinic visit.

FIG. 3 shows blood sampling time points in dose escalation phase B, dose selection phase B, and sentinel-expansion phase C. Abbreviations: A, blood sample for antibody-mediated immunogenicity; C, blood sample for cell-mediated immunogenicity; S, safety blood sample. “Visit” denotes clinic visit.

FIG. 4 illustrates an overview of sequential enrollment and internal safety team safety evaluation in dose-escalation phases A and B. Abbreviations: IST, Internal Safety Team; SMC, Safety Monitoring Committee.

FIG. 5 illustrates an overview of sequential enrollment and safety evaluations in sentinel expansion phase C. Abbreviations: IST, Internal Safety Team; SMC, Safety Monitoring Committee.

FIG. 6 illustrates results of neutralizing antibodies for epithelial cell infection per-protocol set in phase A and phase B seronegative subjects. Solid grey reference line represents the baseline GMT of CMV-seropositive subjects in the study. Days from First Dose: Day 1=1st vaccination; Day 28=1 month after the 1st vaccination; Day 84=1 month after the 2nd vaccination; Day 168=Month 6, prior to the 3rd vaccination; Day 196=1 month after the 3rd vaccination; Day 336=Month 12, 6 months after the 3rd vaccination.

FIG. 7 illustrates results of neutralizing antibodies against epithelial cell infection per-protocol set in phase B by serostatus. Solid grey line represents the baseline GMT of CMV-seropositive subjects in the study. Days from First Dose: Day 1 =1st vaccination; Day 28=1 month after the 1st vaccination; Day 84=1 month after the 2nd vaccination.

FIG. 8 illustrates results of neutralizing antibodies against fibroblast infection per-protocol set in phase A and B seronegative subjects. Solid grey reference line represents the baseline GMT of CMV-seropositive subjects in the study. Days from First Dose: Day 1 =1st vaccination; Day 28=1 month after the 1st vaccination; Day 84=1 month after the 2nd vaccination; Day 168=Month 6, prior to the 3rd vaccination; Day 196=1 month after the 3rd vaccination; Day 336=Month 12, 6 months after the 3rd vaccination.

FIG. 9 illustrates results of neutralizing antibodies fibroblast by serostatus per-protocol set in phase B by serostatus. Solid grey reference line represents the baseline GMT of CMV-seropositive subjects in the study. Days from First Dose: Day 1=1st vaccination; Day 28=1 month after the 1st vaccination; Day 84=1 month after the 2nd vaccination.

FIG. 10 illustrates neutralizing antibody response against epithelial cell infection, by serostatus.

FIG. 11 illustrates neutralizing antibody response against fibroblast infection, by serostatus. *One CMV-seronegative Phase B placebo recipient had nAb titers consistent with seroconversion with onset between the Month 1 and Month 3 timepoints.

FIG. 12 illustrates neutralizing antibody response against epithelial cell infection in CMV-seronegative subjections, per-protocol set. *One CMV-seronegative Phase B placebo recipient had nAb titers consistent with seroconversion with onset between the Month 1 and Month 3 timepoints.

FIG. 13 illustrates neutralizing antibody response against fibroblast infection in CMV-seronegative participants, per protocol set. *One CMV-seronegative Phase B placebo recipient had nAb titers consistent with seroconversion with onset between the Month 1 and Month 3 timepoints.

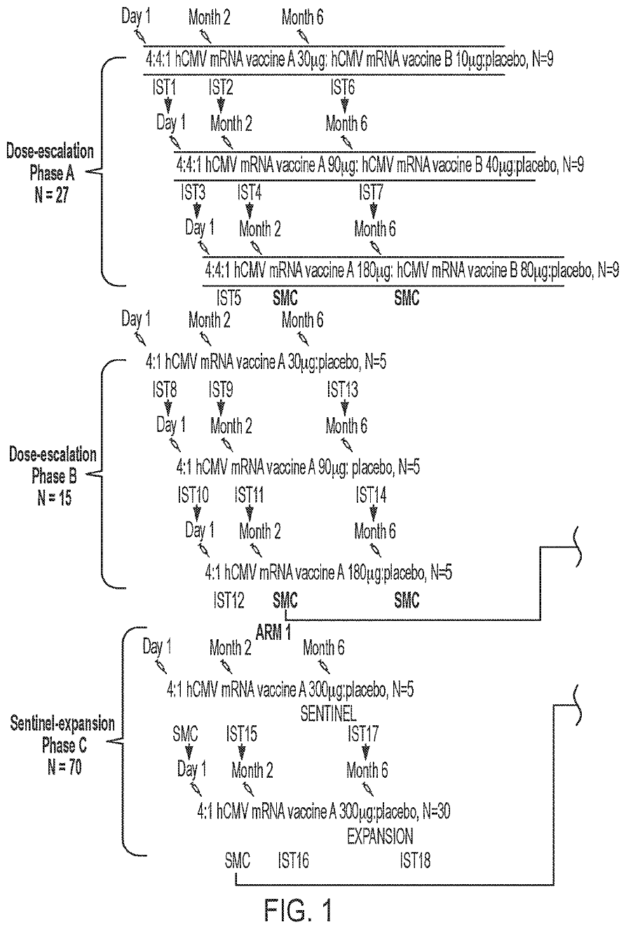

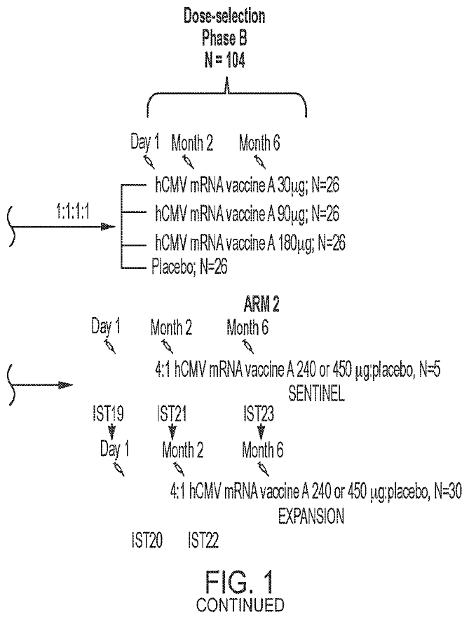

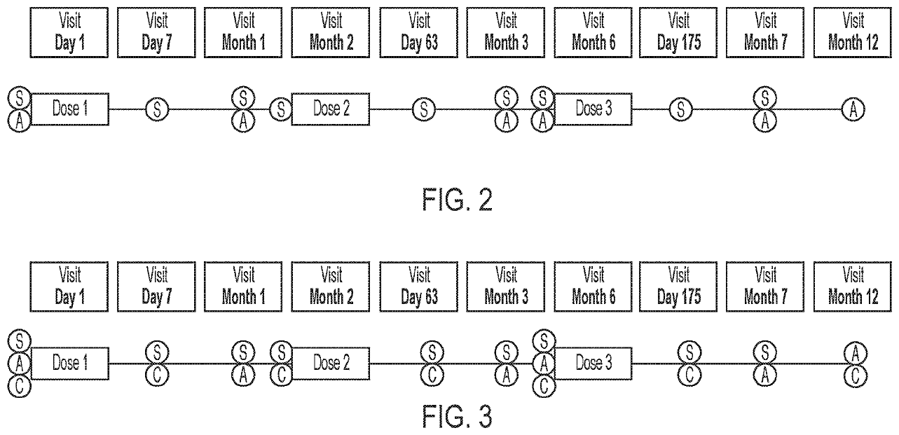

### DETAILED DESCRIPTION

Results from the clinical trial data provided herein demonstrate that hCMV mRNA vaccination of the present disclosure elicited a boost in serum neutralization titers against hCMV infection at all doses levels tested (30, 90, 180 μg, and 300 μg). Serum neutralizing antibody (nAb) geometric mean titer (GMT) increased after each vaccination in a dose-related manner. After the 2^{nd} vaccination in Phase B, nAb GMT against fibroblast infection approached the benchmark of natural CMV infection in the 90 μg and 180 μg treatment groups, and nAb GMT against epithelial cell infection exceeded the benchmark of natural CMV infection in all treatment groups. Neutralizing antibody GMTs were boosted in CMV-seropositive subjects after a single vaccination, which increased further after the second vaccination for nAb GMTs against epithelial cell infection. In Phase A and Phase B CMV-seronegative participants, seroresponses (percentage of subjects with GMTs≥4× baseline titer) were robust through the 2^{nd} vaccination, and continued to be robust through 12 months in Phase A, suggesting sustained antibody responses to hCMV mRNA vaccine A through at least 6 months after the 3^{rd} vaccination.

Further, the overall safety profile of the hCMV mRNA vaccines described herein was similar to that of licensed vaccines (e.g., Gardasil and Shingrix).

### Antigens

Antigens are proteins or polysaccharides capable of inducing an immune response (e.g., causing an immune system to produce antibodies against the antigens). Herein, use of the term antigen encompasses immunogenic proteins and immunogenic fragments that induce (or are capable of inducing) an immune response to hCMV, unless otherwise stated. It should be understood that the term “protein” encompasses peptides and the term “antigen” encompasses antigenic fragments.

HCMV includes several surface glycoproteins that are involved in viral attachment and entry into different cell types. The pentameric complex (PC), composed of gH/gL/UL128/UL130/UL131A (Hahn et al., 2004; Ryckman et al., 2008; Wang and Shenk, 2005b, each of which are incorporated herein by reference), mediates entry into endothelial cells, epithelial cells, and myeloid cells.

HCMV proteins UL128, UL130, and UL131A assemble with gH and gL proteins to form a heterologous pentameric complex, designated gH/gL/UL128-131A, found on the surface of the HCMV. Natural variants and deletion and mutational analyses have implicated proteins of the gH/gL/UL128-131A complex with the ability to infect certain cell types, including for example, endothelial cells, epithelial cells, and leukocytes.

HCMV enters cells by fusing its envelope with either the plasma membrane (fibroblasts) or the endosomal membrane (epithelial and endothelial cells). HCMV initiates cell entry by attaching to the cell surface heparan sulfate proteoglycans using envelope glycoprotein M (gM) or gB. This step is followed by interaction with cell surface receptors that trigger entry or initiate intracellular signaling. The entry receptor function is provided by gH/gL glycoprotein complexes. Different gH/gL complexes are known to facilitate entry into epithelial cells, endothelial cells, or fibroblasts. For example, while entry into fibroblasts requires gH/gL heterodimer, entry into epithelial and endothelial cells requires the pentameric complex gH/gL/UL128/UL130/UL131 in addition to gH/gL. Thus, different gH/gL complexes engage distinct entry receptors on epithelial/endothelial cells and fibroblasts. Receptor engagement is followed by membrane fusion, a process mediated by gB and gH/gL. Early antibody studies have supported critical roles for both gB and gH/gL in hCMV entry. gB is essential for entry and cell spread. gB and gH/gL are necessary and sufficient for cell fusion and thus constitute the “core fusion machinery” of HCMV, which is conserved among other herpesviruses. Thus, the four glycoprotein complexes play a crucial role in viral attachment, binding, fusion and entry into the host cell.

Studies involving the gH/gL/UL128-131A complex have shown that hCMV glycoproteins gB, gH, gL, gM, and gN, as well as UL128, UL130, and UL131A proteins, are immunogenic and involved in the immunostimulatory response in a variety of cell types. Moreover, UL128, UL130, and UL131A genes are relatively conserved among hCMV isolates and therefore represent an attractive target for vaccination. Furthermore, recent studies have shown that antibodies to epitopes within the pentameric gH/gL/UL128-131 complex neutralize entry into endothelial, epithelial, and other cell types, thus blocking the ability of hCMV to infect several cell types.

Without wishing to be bound by any theory, the majority of neutralizing antibodies may be directed against envelope glycoproteins (Britt et al., 1990; Fouts et al., 2012; Macagno et al., 2010; Marshall et al., 1992, incorporated herein by reference), whereas robust T cell responses may be directed against the tegument protein pp65 and nonstructural proteins such as IE1 and IE2 (Blanco-Lobo et al., 2016; Borysiewicz et al., 1988; Kern et al., 2002, incorporated herein by reference).

HCMV envelope glycoprotein complexes (e.g., gH/gL/UL128/UL130/UL131A) represent major antigenic targets of antiviral immune responses. Embodiments of the present disclosure provide RNA (e.g., mRNA) immunogenic compositions (e.g., mRNA vaccines) that include polynucleotides encoding an HCMV antigen, in particular an HCMV antigen from one of the HCMV glycoprotein complexes. Embodiments of the present disclosure provide RNA (e.g., mRNA) immunogenic compositions (e.g., mRNA vaccines) that include at least one polynucleotide encoding at least one hCMV antigenic polypeptide. The HCMV RNA immunogenic compositions (e.g., mRNA vaccines) provided herein may be used to induce a balanced immune response, comprising both cellular and humoral immunity, without many of the risks associated with DNA vaccines and live attenuated vaccines.

The entire contents of International Application No. PCT/US2015/027400 (WO 2015/164674), entitled “Nucleic Acid Vaccines,” International Application No. PCT/US2016/058310 (WO2017/070613), entitled “HUMAN CYTOMEGALOVIRUS VACCINE,” International Application No. PCT/US2017/057748 (WO2018/075980), entitled “HUMAN CYTOMEGALOVIRUS VACCINE,” U.S. Pat. No. 10,064,935, entitled “HUMAN CYTOMEGALOVIRUS VACCINE,” U.S. Pat. No. 10,383,937, entitled “HUMAN CYTOMEGALOVIRUS VACCINE,” U.S. Pat. No. 10,064,935, entitled “HUMAN CYTOMEGALOVIRUS VACCINE,” and U.S. Pat. No. 10,716,846, entitled “HUMAN CYTOMEGALOVIRUS VACCINE” are incorporated herein by reference.

The hCMV antigens of the immunogenic compositions (e.g., vaccines such as mRNA vaccines) of the present disclosure are provided in Table 5 herein. In some embodiments, the hCMV immunogenic composition (e.g., mRNA vaccine) comprises: (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB. In some embodiments, the weight ratio of the mRNA encoding hCMV gH, gL, UL128, UL130, UL131A, and gB proteins in the vaccine composition is 1:1:1:1:1:1. In some embodiments, the hCMV immunogenic composition (e.g., vaccine) components comprise the sequences provided in Table 5.

In some embodiments, the hCMV immunogenic composition (e.g., mRNA vaccine) comprises an mRNA polynucleotide comprising an open reading frame encoding a hCMV mutant pp65 polypeptide (designated herein as pp65mut).

In some embodiments, the hCMV immunogenic composition (e.g., mRNA vaccine) comprises: (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; (f) a mRNA polynucleotide comprising an open reading frame encoding a hCMV gB; and (g) a mRNA polynucleotide comprising an open reading frame encoding a hCMV mutant pp65 polypeptide.

In some embodiments, the hCMV gH polypeptide comprises the amino acid sequence of SEQ ID NO: 19. In some embodiments, the hCMV gL polypeptide comprises the amino acid sequence of SEQ ID NO: 20. In some embodiments, the hCMV UL128 polypeptide comprises the amino acid sequence of SEQ ID NO: 16. In some embodiments, the hCMV UL130 polypeptide comprises the amino acid sequence of SEQ ID NO: 17. In some embodiments, the hCMV UL131A polypeptide comprises the amino acid sequence of SEQ ID NO: 18. In some embodiments, the hCMV gB polypeptide comprises the amino acid sequence of SEQ ID NO: 15.

In some embodiments, the mRNA encoding the hCMV gH polypeptide comprises an open reading frame (ORF) of the nucleotide sequence of SEQ ID NO: 11. In some embodiments, the mRNA encoding the hCMV gL polypeptide comprises an open reading frame (ORF) of the nucleotide sequence of SEQ ID NO: 12. In some embodiments, the mRNA encoding the hCMV UL128 polypeptide comprises an open reading frame (ORF) of the nucleotide sequence of SEQ ID NO: 8. In some embodiments, the mRNA encoding the hCMV UL130 polypeptide comprises an open reading frame (ORF) of the nucleotide sequence of SEQ ID NO: 9. In some embodiments, the mRNA encoding the hCMV UL131A polypeptide comprises an open reading frame (ORF) of the nucleotide sequence of SEQ ID NO: 10. In some embodiments, the mRNA encoding the hCMV gB polypeptide comprises an open reading frame (ORF) of the nucleotide sequence of SEQ ID NO: 7.

In some embodiments, the mRNA encoding the hCMV gH polypeptide comprises the nucleotide sequence of SEQ ID NO: 5. In some embodiments, the mRNA encoding the hCMV gL polypeptide comprises an open reading frame (ORF) of the nucleotide sequence of SEQ ID NO: 6. In some embodiments, the mRNA encoding the hCMV UL128 polypeptide comprises the nucleotide sequence of SEQ ID NO: 2. In some embodiments, the mRNA encoding the hCMV UL130 polypeptide comprises the nucleotide sequence of SEQ ID NO: 3. In some embodiments, the mRNA encoding the hCMV UL131A polypeptide comprises the nucleotide sequence of SEQ ID NO: 4. In some embodiments, the mRNA encoding the hCMV gB polypeptide comprises the nucleotide sequence of SEQ ID NO: 1.

In some embodiments, the hCMV pp65mut polypeptide comprises the amino acid sequence of SEQ ID NO: 24. In some embodiments, the mRNA encoding the hCMV pp65mut polypeptide comprises an open reading frame (ORF) of the nucleotide sequence of SEQ ID NO: 23. In some embodiments, the mRNA encoding the hCMV pp65mut polypeptide comprises the nucleotide sequence of SEQ ID NO: 21.

In some embodiments, the aforementioned mRNAs may further comprise a 5′ cap (e.g., 7mG(5′)ppp(5′)NlmpNp), a polyA tail (e.g., ˜100 nucleotides), or a 5′ cap and a polyA tail.

It should be understood that the hCMV mRNA components of the immunogenic compositions (e.g., mRNA vaccines) of the present disclosure may comprise a signal sequence. It should also be understood that the hCMV mRNA components of the immunogenic compositions (e.g., mRNA vaccines) of the present disclosure may include any 5′ untranslated region (UTR) and/or any 3′ UTR. Exemplary UTR sequences are provided in Table 5; however, other UTR sequences (e.g., of the prior art) may be used or exchanged for any of the UTR sequences described herein. UTRs may also be omitted from the vaccine constructs provided herein.

### Nucleic Acids

The hCMV immunogenic compositions (e.g., mRNA vaccines) of the present disclosure comprise at least one (one or more) ribonucleic acid (RNA) having an open reading frame encoding at least one hCMV antigen. In some embodiments, the RNA is a messenger RNA (mRNA) having an open reading frame encoding at least one hCMV antigen. In some embodiments, the RNA (e.g., mRNA) further comprises a (at least one) 5′ UTR, 3′ UTR, a polyA tail and/or a 5′ cap.

Nucleic acids comprise a polymer of nucleotides (nucleotide monomers), also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an a-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any ribonucleic acid that encodes a (at least one) protein (a naturally-occurring, non-naturally-occurring, or modified polymer of amino acids) and can be translated to produce the encoded protein in vitro, in vivo, in situ or ex vivo. The skilled artisan will appreciate that, except where otherwise noted, nucleic acid sequences set forth in the instant application may recite “T”s in a representative DNA sequence but where the sequence represents

RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in a vaccine of the present disclosure.

### Variants

In some embodiments, the hCMV immunogenic composition (e.g., mRNA vaccine) of the present disclosure comprises mRNAs encoding an hCMV antigen variant. Antigen or other polypeptide variants refers to molecules that differ in their amino acid sequence from a wild-type, native or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, an hCMV immunogenic composition (e.g., mRNA vaccine) comprises an mRNA ORF having a nucleotide sequence identified by any one of the sequences provided herein (see e.g., Table 5), or having a nucleotide sequence at least 80%, at least 85%, at least 90%, at least 95%, at least 96%, at least 97%, at least 98%, or at least 99% identical (including all values in between) to a nucleotide sequence identified by any one of the sequence provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T.F. & Waterman, M.S. (1981) “Identification of common molecular subsequences.” J. Mol. Biol. 147:195-197). A general global alignment technique based on dynamic programming is the Needleman-Wunsch algorithm (Needleman, S.B. & Wunsch, C.D. (1970) “A general method applicable to the search for similarities in the amino acid sequences of two proteins.” J. Mol. Biol. 48:443-453). More recently a Fast Optimal

Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of hCMV antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the hCMV pathogen. In addition to variants that are identical to the reference protein but are truncated, in some embodiments, an antigen includes 2, 3, 4, 5, 6, 7, 8, 9, 10, or more mutations, relative to any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, the hCMV immunogenic composition (e.g., mRNA vaccine) includes at least one RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5¹)ppp(5′)G-2′-0-methyl. Cap 2 structure may be generated from the Cap 1 structure followed by the 2′-0-methylation of the 5′-antepenultimate nucleotide using a 2′-O methyl-transferase. Cap 3 structure may be generated from the Cap 2 structure followed by the 2¹-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, the hCMV immunogenic composition (e.g., mRNA vaccine) includes one or more stabilizing elements. Stabilizing elements may include for instance a histone stem-loop. A 32 kDa stem-loop binding protein (SLBP) has been reported. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the hCMV immunogenic composition (e.g., mRNA vaccine) includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine: guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, the hCMV immunogenic composition (e.g., mRNA vaccine) does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

The hCMV immunogenic composition (e.g., mRNA vaccine) may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, the hCMV immunogenic composition (e.g., mRNA vaccine) has one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

### Signal Peptides

In some embodiments, an hCMV immunogenic composition (e.g., mRNA vaccine) comprises an mRNA having an ORF that encodes a signal peptide fused to the hCMV antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane. A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than hCMV antigens in nature) are known in the art and can be tested for desired properties and then incorporated into a nucleic acid of the disclosure. In some embodiments, the signal peptide may comprise one of the following sequences: MDSKGSSQKGSRLLLLLVVSNLLLPQGVVG (SEQ ID NO: 25), MDWTWILFLVAAATRVHS (SEQ ID NO: 26); METPAQLLFLLLLWLPDTTG (SEQ ID NO: 22); MLGSNSGQRVVFTILLLLVAPAYS (SEQ ID NO: 27); MKCLLYLAFLFIGVNCA (SEQ ID NO: 28); MWLVSLAIVTACAGA (SEQ ID NO: 29).

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a hCMV antigen). In some embodiments, a codon optimized sequence shares less than 90% sequence identity to a naturally-occurring or wild-type sequence (e.g., a naturally-occurring or wild-type mRNA sequence encoding a hCMV antigen). In some embodiments, a codon optimized sequence shares less than 85% sequence identity to a naturally-occurring or wild-type sequence (e.g., a naturally-occurring or wild-type mRNA sequence encoding a hCMV antigen). In some embodiments, a codon optimized sequence shares less than 80% sequence identity to a naturally-occurring or wild-type sequence (e.g., a naturally-occurring or wild-type mRNA sequence encoding a hCMV antigen). In some embodiments, a codon optimized sequence shares less than 75% sequence identity to a naturally-occurring or wild-type sequence (e.g., a naturally-occurring or wild-type mRNA sequence encoding hCMV antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a hCMV antigen). In some embodiments, a codon optimized sequence shares between 65% and 75% or about 80% sequence identity to a naturally-occurring or wild-type sequence (e.g., a naturally-occurring or wild-type mRNA sequence encoding a hCMV antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a hCMV antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, at least one RNA (e.g., mRNA) of an hCMV mRNA vaccine of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The hCMV immunogenic compositions (e.g., mRNA vaccines) of the present disclosure comprise, in some embodiments, at least one nucleic acid (e.g., RNA) having an open reading frame encoding at least one hCMV antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art. In some embodiments, a naturally-occurring modified nucleotide or nucleoside of the disclosure is one as is generally known or recognized in the art. Non-limiting examples of such naturally occurring modified nucleotides and nucleosides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/U52014/058891; PCT/U52014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e 1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (w) substitutions at one or more or all uridine positions of the nucleic acid.

In some embodiments, a mRNA of the disclosure comprises pseudouridine (w) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 30), where R is a purine (adenine or guanine) three bases upstream of the start codon (AUG), which is followed by another ‘G’.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (US8278063; US9012219), human cytochrome b-245 a polypeptide, and hydroxysteroid (17b) dehydrogenase, and Tobacco etch virus (U.S. Pat. Nos. 8,278,063, 9,012,219). CMV immediate-early 1 (TE1) gene (US20140206753, WO2013/185069), the sequence GGGAUCCUACC (SEQ ID NO: 31) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a nucleotide sequence of SEQ ID NO: 13.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) (SEQ ID NO: 32) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

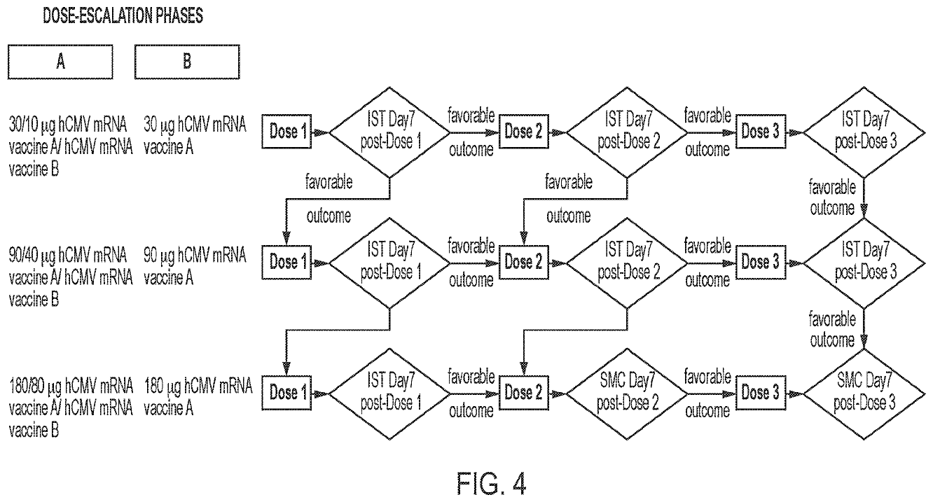

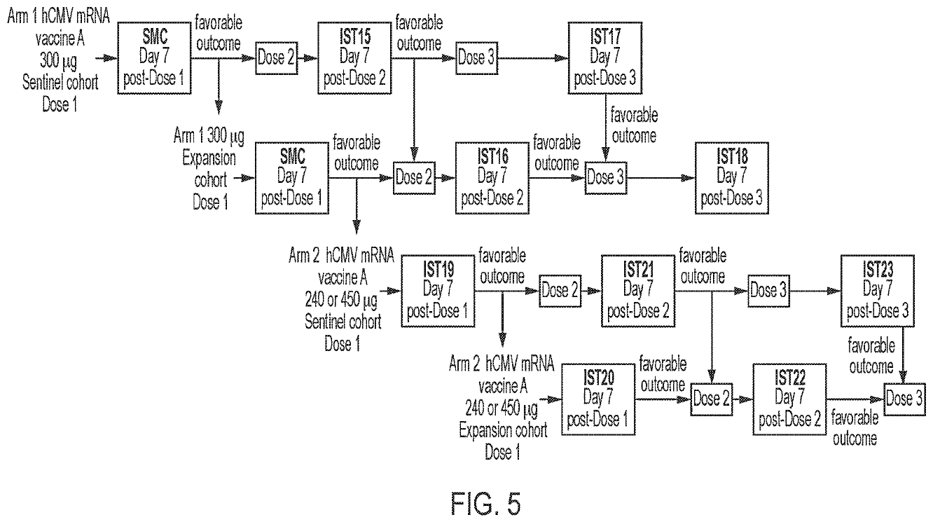

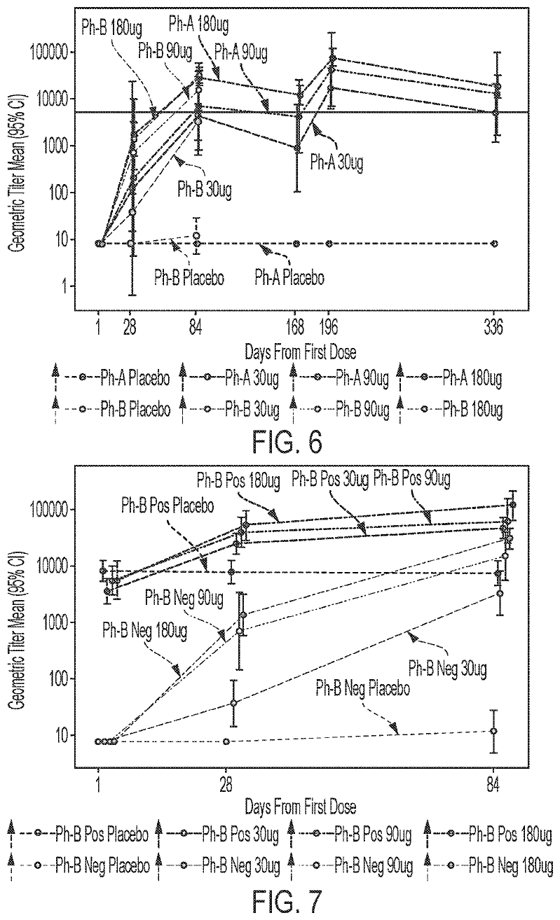

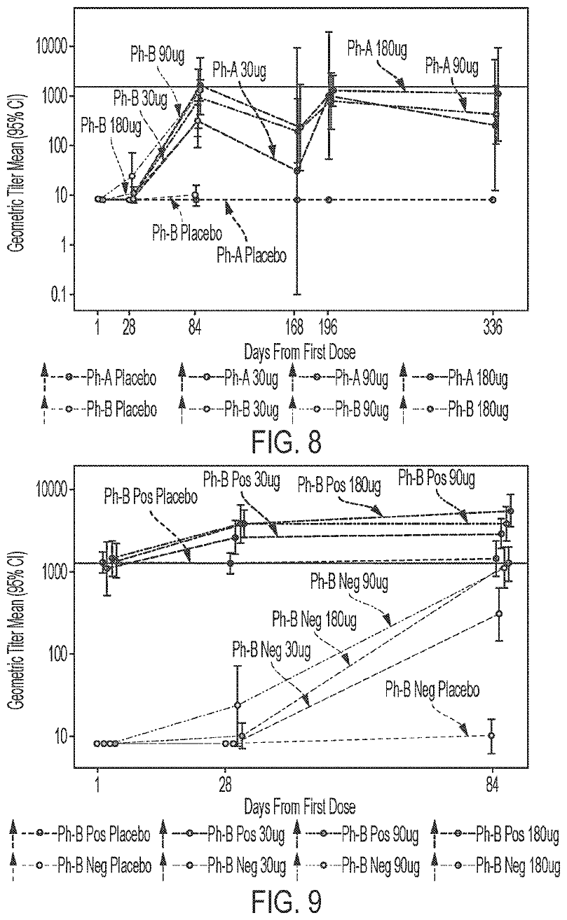

3′ UTRs may be heterologous or synthetic. With respect to 3′ UTRs, globin UTRs, including Xenopus (3-globin UTRs and human (3-globin UTRs are known in the art (U.S. Pat. Nos. 8,278,063, 9,012,219, US20110086907). A modified (3-globin construct with enhanced stability in some cell types by cloning two sequential human (3-globin 3′UTRs head to tail has been developed and is well known in the art (US2012/0195936, WO2014/071963). In addition a2-globin, a 1 -globin, UTRs and mutants thereof are also known in the art (WO2015101415, WO2015024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014152774), rabbit p globin and hepatitis B virus (HBV), a-globin 3′ UTR and Viral VEEV 3′ UTR sequences are also known in the art. In some embodiments, the sequence UUUGAAUU (WO2014144196) is used. In some embodiments, 3′ UTRs of human and mouse ribosomal protein are used. Other examples include rps9 3′UTR (WO2015101414), FIG 4 (WO2015101415), and human albumin 7 (WO2015101415).

In some embodiments, a 3′ UTR of the present disclosure comprises a nucleotide sequence of SEQ ID NO: 14.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No.20100293625 and PCT/US2014/069155, herein incorporated by reference in its entirety.

In vitro Transcription of RNA

cDNA encoding the polynucleotides described herein may be transcribed using an in vitro transcription (IVT) system. In vitro transcription of RNA is known in the art and is described in International Publication WO/2014/152027, which is incorporated by reference herein in its entirety.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to hCMV mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

When RNA transcripts are being generated, the 5′ UTR may comprise a promoter sequence. Such promoter sequences are known in the art. It should be understood that such promoter sequences will not be present in a vaccine of the disclosure.

A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

In some embodiments, the RNA transcript is capped via enzymatic capping. In some embodiments, the RNA comprises 5′ terminal cap, for example, 7mG(5′)ppp(5′)NlmpNp.

### Lipid Nanoparticles (LNPs)

In some embodiments, the hCMV immunogenic compositions (mRNA vaccines) of the disclosure are formulated in one or more lipid nanoparticles (LNPs). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entireties.

Vaccines of the present disclosure are typically formulated in lipid nanoparticles. In some embodiments, the lipid nanoparticle comprises at least one ionizable cationic lipid, at least one non-cationic lipid, at least one sterol, and/or at least one polyethylene glycol (PEG)-modified lipid.

The lipid nanoparticles of the present disclosure are comprised of a mixture of lipids and the amounts are measured according to the mole faction or the mole percent of each lipid component in the lipid nanoparticle. Mole percent is obtained by multiplying the mole fraction by 100%. The mRNA and any water are not represented where the lipid mixture is accounted for numerically.

In some embodiments, the lipid nanoparticle comprises a mixture of lipids comprising 20-60 mol % ionizable cationic lipid. For example, the lipid nanoparticle may comprise a mole percent of 20-50 mol %, 20-40 mol %, 20-30 mol %, 30-60 mol %, 30-50 mol %, 30-40 mol %, 40-60 mol %, 40-50 mol %, or 50-60 mol % ionizable cationic lipid. In some embodiments, the lipid nanoparticle comprises 20 mol %, 30 mol %, 40 mol %, 50 mol %, or 60 mol % ionizable cationic lipid.

In some embodiments, the lipid nanoparticle comprises a mixture of lipids comprising 5-25 mol % non-cationic lipid. For example, the lipid nanoparticle may comprise a non-cationic lipid comprising 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises a mixture of lipids comprising 5 mol %, 10 mol %, 15 mol %, 20 mol %, or 25 mol % non-cationic lipid.

In some embodiments, the lipid nanoparticle comprises a mixture of lipids comprising 25-55 mol % sterol. For example, the lipid nanoparticle may comprise a sterol comprising 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises a mole percent of 25 mol %, 30 mol %, 35 mol %, 40 mol %, 45 mol %, 50 mol %, or 55 mol % sterol.

In some embodiments, the lipid nanoparticle comprises a mixture of lipids comprising 0.5-15 mol % PEG-modified lipid. For example, the lipid nanoparticle may comprise a mole percent of 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol % PEG-modified lipid. In some embodiments, the lipid nanoparticle comprises a mole percent of 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the lipid nanoparticle comprises a mixture of lipids comprising 49 mol % ionizable cationic lipid, 38.5 mol % cholesterol, 10 mol % DSPC, and 2.5 mol % DMG-PEG. In some embodiments, the lipid nanoparticle comprises a mixture of lipids comprising 48 mol % ionizable cationic lipid, 38.5 mol % cholesterol, 11 mol % DSPC, and 2.5 mol % DMG-PEG. In some embodiments, the lipid nanoparticle comprises a mixture of lipids comprising 47 mol % ionizable cationic lipid, 38.5 mol % cholesterol, 11.5 mol % DSPC, and 3 mol % DMG-PEG.

In some embodiments, an ionizable cationic lipid of the disclosure comprises a compound having structure:

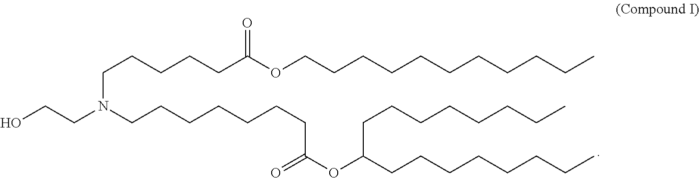 [SMILES: CCCCCCCCCCCOC(=O)CCCCCN(CCO)CCCCCCCC(=O)OC(CCCCCCCC)CCCCCCCC]

In some embodiments, an ionizable cationic lipid of the disclosure comprises a compound having structure:

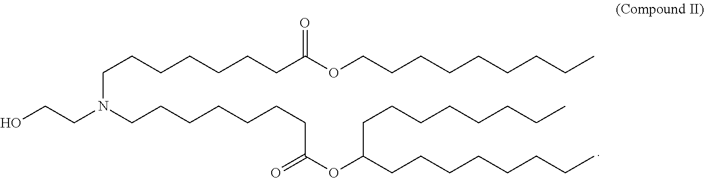 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC),1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is DMG-PEG.

In some embodiments, the lipid nanoparticle comprises 45-55 mole percent ionizable cationic lipid. For example, lipid nanoparticle may comprise 45, 46, 47, 48, 49, 50, 51, 52, 53, 54, or 55 mole percent ionizable cationic lipid.

In some embodiments, the lipid nanoparticle comprises 5-15 mole percent DSPC. For example, the lipid nanoparticle may comprise 5, 6, 7, 8, 9, 10, 11, 12, 13, 14, or 15 mole percent DSPC.

In some embodiments, the lipid nanoparticle comprises 35-40 mole percent cholesterol. For example, the lipid nanoparticle may comprise 35, 36, 37, 38, 39, or 40 mole percent cholesterol.

In some embodiments, the lipid nanoparticle comprises 1-2 mole percent DMG-PEG. For example, the lipid nanoparticle may comprise 1, 1.5, or 2 mole percent DMG-PEG.

In some embodiments, the lipid nanoparticle comprises 50 mole percent ionizable cationic lipid, 10 mole percent DSPC, 38.5 mole percent cholesterol, and 1.5 mole percent DMG-PEG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Multivalent Vaccines

The hCMV immunogenic compositions (e.g., mRNA vaccines), as provided herein, may include mRNA or multiple mRNAs encoding two or more antigens of the same or different hCMV species. In some embodiments, the hCMV immunogenic composition (e.g., mRNA vaccine) includes an RNA or multiple RNAs encoding two or more antigens. In some embodiments, the mRNA of a hCMV immunogenic composition (e.g., mRNA vaccine) may encode 1, 2, 3, 4, 5, 6, 7, 8, 9, 10, 11, 12, or more antigens.

In some embodiments, the hCMV immunogenic composition (mRNA vaccine) comprises at least one RNA encoding an hCMV gH, an hCMV gL, an hCMV UL128, an hCMV UL130, an hCMV UL131A, and an hCMV gB. In some embodiments, the hCMV immunogenic composition (e.g., mRNA vaccine) comprises at least one RNA encoding a hCMV pp65mut. In some embodiments, the hCMV immunogenic composition (e.g., mRNA vaccine) comprises at least one RNA encoding an hCMV gH, an hCMV gL, an hCMV UL128, an hCMV UL130, an hCMV UL131A, an hCMV gB, and a hCMV pp65mut.

In some embodiments, two or more different RNAs (e.g., mRNAs) encoding antigens may be formulated in the same lipid nanoparticle. In other embodiments, two or more different RNAs encoding antigens may be formulated in separate lipid nanoparticles (e.g., each RNA formulated in a single lipid nanoparticle). The lipid nanoparticles may then be combined and administered as a single vaccine composition (e.g., comprising multiple RNA encoding multiple antigens) or may be administered separately.

### Pharmaceutical Formulations

Provided herein are compositions (e.g., pharmaceutical compositions), methods, kits and reagents for prevention or treatment of hCMV in humans and other mammals, for example. hCMV mRNA vaccines can be used as therapeutic or prophylactic agents. They may be used in medicine to prevent and/or treat infectious disease.

In some embodiments, the hCMV immunogenic compositions (e.g., mRNA vaccines) containing mRNA as described herein can be administered to a subject (e.g., a mammalian subject, such as a human subject), and the RNA polynucleotides are translated in vivo to produce an antigenic polypeptide (antigen).

An “effective amount” of a hCMV immunogenic composition (e.g., mRNA vaccine) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a hCMV immunogenic composition (e.g., mRNA vaccine) provides an induced or boosted immune response as a function of antigen production in the cells of the subject. In some embodiments, an effective amount of the hCMV immunogenic composition (e.g., mRNA vaccine) containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, immunological compositions (e.g., RNA vaccines including polynucleotides and their encoded polypeptides) in accordance with the present disclosure may be used for treatment or prevention of hCMV infection. The hCMV immunological composition (e.g., mRNA vaccine) may be administered prophylactically or therapeutically as part of an active immunization scheme to healthy individuals or early in infection during the incubation phase or during active infection after onset of symptoms. In some embodiments, the amount of RNA vaccines of the present disclosure provided to a cell, a tissue or a subject may be an amount effective for immune prophylaxis.

The hCMV immunological composition (e.g., mRNA vaccine) may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 4 months, 5 months, 6 months or 1 year.

In some embodiments, the hCMV immunological compositions (e.g., mRNA vaccine) may be administered intramuscularly (e.g., to deltoid muscle), intranasally or intradermally, similarly to the administration of inactivated vaccines known in the art.

The hCMV immunogenic composition (e.g., mRNA vaccine) may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including the hCMV immunogenic composition (e.g., mRNA vaccine) and/or complexes optionally in combination with one or more pharmaceutically acceptable excipients.

The hCMV immunogenic composition (e.g., mRNA vaccine) may be formulated or administered alone or in conjunction with one or more other components. For instance, the hCMV immunogenic composition (e.g., mRNA vaccine) may comprise other components including, but not limited to, adjuvants.

In some embodiments, the hCMV immunogenic composition (e.g., mRNA vaccine) does not include an adjuvant (they are adjuvant free). In some embodiments, the hCMV immunogenic composition (e.g., mRNA vaccine) includes an adjuvant. Any known adjuvant suitable for use in vaccines may be used. In some embodiments, the hCMV immunogenic composition (e.g., mRNA vaccine) includes an MF59 adjuvant system (e.g., as described in O′Hagan et al., Expert Rev Vaccines. 2007 Oct; 6(5):699-710, incorporated herein by reference).

The hCMV immunogenic composition (e.g., mRNA vaccine) may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, the hCMV immunogenic compositions (e.g., mRNA vaccines) are administered to humans, such as human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single-or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, the hCMV immunogenic composition (e.g., mRNA vaccine) is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the hCMV immunogenic composition (e.g., mRNA vaccine) (e.g., for transplantation into a subject), hyaluronidase, nanoparticle mimics and combinations thereof.

### Dosing/Administration

Provided herein are compositions (e.g., pharmaceutical compositions), methods, kits and reagents for prevention and/or treatment of hCMV infection in humans and other mammals. The hCMV immunogenic composition (e.g., mRNA vaccine) can be used as therapeutic or prophylactic agents. In some aspects, the RNA vaccines of the disclosure are used to provide prophylactic protection from hCMV. In some aspects, the RNA vaccines of the disclosure are used to treat a hCMV infection. In some embodiments, the hCMV immunogenic composition (e.g., mRNA vaccine) of the present disclosure is used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, the hCMV immunogenic composition (e.g., mRNA vaccine) is administered to a subject (e.g., a mammalian subject, such as a human subject) in an effective amount to induce an antigen-specific immune response. The RNA encoding the hCMV antigen is expressed and translated in vivo to produce the antigen, which then stimulates an immune response in the subject. The subject may be hCMV seropositive (e.g., has previously had a natural hCMV infection) or hCMV seronegative (e.g., has not previously had a natural hCMV infection) prior of being administered the hCMV immunogenic composition (e.g., mRNA vaccine).

Prophylactic protection from hCMV can be achieved following administration of the hCMV immunogenic composition (e.g., mRNA vaccine) of the present disclosure. Vaccines can be administered once, twice, three times, four times or more but it is likely sufficient to administer the vaccine once (optionally followed by one or more boosters). It is possible, although less desirable, to administer the vaccine to an infected individual to achieve a therapeutic response. Dosing may need to be adjusted accordingly.

A method of eliciting an immune response in a subject against hCMV is provided in aspects of the present disclosure. The method involves administering to the subject a hCMV immunogenic composition (e.g., mRNA vaccine) comprising at least one RNA (e.g., mRNA) having an open reading frame encoding at least one hCMV antigen, thereby inducing in the subject an immune response specific to a hCMV antigen, wherein anti-antigen antibody titer in the subject is increased following vaccination relative to anti-antigen antibody titer in a subject vaccinated with a prophylactically effective dose of a traditional vaccine against the hCMV. An “anti-antigen antibody” is a serum antibody the binds specifically to the antigen.

In some embodiments, a prophylactically effective dose is an effective dose that prevents infection with the virus at a clinically acceptable level. In some embodiments, the effective dose is a dose listed in a package insert for the vaccine. In some embodiments, an effective dose is sufficient to produce detectable levels of hCMV antigen (e.g., gH, gL, UL128, UL130, UL131A and/or gB polypeptide) as measured in serum of the subject administered the hCMV immunogenic composition (e.g., mRNA vaccine) at 1-72 hours (e.g., 1-72 hours, 1-60 hours, 1-45 hours, 1-30 hours, 1-15 hours, 15-72 hours, 15-60 hours, 15-45 hours, 15-30 hours, 30-72 hours, 30-60 hours, 30-45 hours, 45-72 hours, 45-60 hours, or 60-72 hours) post administration. In some embodiments, the effective dose is sufficient to produce neutralization titer produced by neutralizing antibody against the hCMV antigen (e.g., gH, gL, UL128, UL130, UL131A and/or gB polypeptide) as measured in serum of the subject administered the hCMV immunogenic composition (e.g., mRNA vaccine) at 1-72 hours (e.g., 1-72 hours, 1-60 hours, 1-45 hours, 1-30 hours, 1-15 hours, 15-72 hours, 15-60 hours, 15-45 hours, 15-30 hours, 30-72 hours, 30-60 hours, 30-45 hours, 45-72 hours, 45-60 hours, or 60-72 hours) post administration.

A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the hCMV or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the hCMV or an unvaccinated subject.

A method of eliciting an immune response in a subject against hCMV is provided in other aspects of the disclosure. The method involves administering to the subject the hCMV mRNA vaccine comprising at least one RNA polynucleotide having an open reading frame encoding at least one hCMV antigen, thereby inducing in the subject an immune response specific to hCMV antigen, wherein the immune response in the subject is equivalent to an immune response in a subject vaccinated with a traditional vaccine against the hCMV at 2 times to 100 times the dosage level relative to the RNA vaccine.

In some embodiments, the immune response in the subject is equivalent to an immune response in a subject vaccinated with a traditional vaccine at twice the dosage level relative to the hCMV immunogenic composition (e.g., mRNA vaccine). In some embodiments, the immune response in the subject is equivalent to an immune response in a subject vaccinated with a traditional vaccine at three times the dosage level relative to the hCMV immunogenic composition (e.g., mRNA vaccine). In some embodiments, the immune response in the subject is equivalent to an immune response in a subject vaccinated with a traditional vaccine at 4 times, 5 times, 10 times, 50 times, or 100 times the dosage level relative to the hCMV immunogenic composition (e.g., mRNA vaccine). In some embodiments, the immune response in the subject is equivalent to an immune response in a subject vaccinated with a traditional vaccine at 10 times to 1000 times the dosage level relative to the hCMV immunogenic composition (e.g., mRNA vaccine). In some embodiments, the immune response in the subject is equivalent to an immune response in a subject vaccinated with a traditional vaccine at 100 times to 1000 times the dosage level relative to the hCMV immunogenic composition (e.g., mRNA vaccine).

In other embodiments, the immune response is assessed by determining [protein] antibody titer in the subject. In other embodiments, the ability of serum or antibody from an immunized subject is tested for its ability to neutralize viral uptake or reduce hCMV transformation of human B lymphocytes. In other embodiments, the ability to promote a robust T cell response(s) is measured using art recognized techniques.

Other aspects the disclosure provide methods of eliciting an immune response in a subject against hCMV by administering to the subject the hCMV immunogenic composition (e.g., mRNA vaccine) comprising at least one RNA polynucleotide having an open reading frame encoding at least one hCMV antigen, thereby inducing in the subject an immune response specific to hCMV antigen, wherein the immune response in the subject is induced 2 days to 10 weeks earlier relative to an immune response induced in a subject vaccinated with a prophylactically effective dose of a traditional vaccine against hCMV. In some embodiments, the immune response in the subject is induced in a subject vaccinated with a prophylactically effective dose of a traditional vaccine at 2 times to 100 times the dosage level relative to the RNA vaccine.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against a hCMV by administering to the subject the hCMV immunogenic composition (e.g., mRNA vaccine) having an open reading frame encoding a first antigen, wherein the RNA polynucleotide does not include a stabilization element, and wherein an adjuvant is not co-formulated or co-administered with the vaccine.

The hCMV immunogenic composition (e.g., mRNA vaccine) may be administered by any route which results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. The hCMV mRNA vaccine is typically formulated in dosage unit form for ease of administration and uniformity of dosage. It will be understood, however, that the total daily usage of the hCMV mRNA vaccine may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, the hCMV immunogenic composition (e.g., mRNA vaccine A) is administered at a dose of about 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg , 42 μg, 43 μg , 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg , 82 μg , 83 μg , 84 μg, 85 μg, 86 μg, 87 μg, 88 μg, 89 μg, 90 μg, 95 μg, 100 μg, 110 μg, 120 μg, 130 μg, 140 μg, 150 μg, 160 μg, 170 μg, 180 μg, 190 μg, 200 μg, 250 μg, 300 μg, 350 μg, 400 μg, 450 μg, or 500 μg, including all values in between. In some embodiments, only one dose is administered, while in other embodiments, multiple doses are administered. In embodiments wherein multiple doses are administered, the does between the first dose and a subsequent dose can be the same or different.

In some embodiments, the effective amount of the hCMV immunogenic composition (e.g., mRNA vaccine A, including mRNAs encoding gH/gL/UL128/UL130/UL131A/gB), as provided herein, may be as low as 90 μg, administered for example as a single dose or as three 30 μg doses. In some embodiments, the effective amount of hCMV immunogenic composition (e.g., mRNA vaccine A) is a single dose of 25 μg -500 μg or 30 μg -450 μg. For example, the effective amount of hCMV immunogenic composition (e.g., mRNA vaccine A) may be a single dose of 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μs, 23 μs, 24 μg 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg , 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μs, 65 μs, 66 μs, 67 μs, 68 μg, 69 μg, 70 μg, 71 μg, 72 μg, 73 μg, 74 μg, 75 μg, 76 μg, 77 μg, 78 μg, 79 μg, 80 μg, 81 μg , 82 μg, 83 μg , 84 μg, 85 μg, 86 μg, 87 μg, 88 μg, 89 μg, 90 μg, 95 μg, 100 μg, 110 μg, 120 μg, 130 μg, 140 μg, 150 μg, 160 μg, 170 μg, 180 μg, 190 μg, 200 μg, 250 μg, 300 μg, 350 μg, 400 μg, 450 μg, or 500 μg, including all values in between.

In some embodiments, the effective amount of hCMV immunogenic composition (e.g., mRNA vaccine A) is a single dose of 30 μg -180 μg. In some embodiments, the effective amount of hCMV immunogenic composition (e.g., mRNA vaccine A) is a single dose of 30 μg. In some embodiments, the effective amount of hCMV immunogenic composition (e.g., mRNA vaccine A) is a single dose of 90 μg. In some embodiments, the effective amount of hCMV immunogenic composition (e.g., mRNA vaccine A) is a single dose of 180 μg. In some embodiments, the effective amount of hCMV immunogenic composition (e.g., mRNA vaccine A) is a single dose of 300 μg -450 μg. In some embodiments, the effective amount of hCMV immunogenic composition (e.g., mRNA vaccine A) is a single dose of 300 μg. In some embodiments, the effective amount of hCMV immunogenic composition (e.g., mRNA vaccine A) is a single dose of 450 μg.

In some embodiments, the effective amount of hCMV immunogenic composition (e.g., mRNA vaccine A) is 2 doses of 25 μg -500 μg or 30 μg -450 μg. For example, the effective amount of hCMV immunogenic composition (e.g., mRNA vaccine A) may be 2 doses of 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg , 42 μg, 43 μg , 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg , 82 μg , 83 μg , 84 μg, 85 μg, 86 μg, 87 μg, 88 μg, 89 μg, 90 μg, 95 μg, 100 μg, 110 μg, 120 μg, 130 μg, 140 μg, 150 μg, 160 μg, 170 μg, 180 μg, 190 μg, 200 μg, 250 μg, 300 μg, 350 μg, 400 μg, 450 μg, or 500 μg, including all values in between.

In some embodiments, the effective amount of hCMV immunogenic composition (e.g., mRNA vaccine A) is 2 doses of 30 μg -180 μg. In some embodiments, the effective amount of hCMV immunogenic composition (e.g., mRNA vaccine A) is 2 doses of 30 μg. In some embodiments, the effective amount of hCMV immunogenic composition (e.g., mRNA vaccine A) is 2 doses of 90 μg. In some embodiments, the effective amount of hCMV immunogenic composition (e.g., mRNA vaccine A) is 2 doses of 180 μg. In some embodiments, the effective amount of hCMV immunogenic composition (e.g., mRNA vaccine A) is 2 doses of 300 μg -450 μg. In some embodiments, the effective amount of hCMV immunogenic composition (e.g., mRNA vaccine A) is 2 doses of 300 μg. In some embodiments, the effective amount of hCMV immunogenic composition (e.g., mRNA vaccine A) is 2 doses of 450 μg.

In some embodiments, the effective amount of hCMV immunogenic composition (e.g., mRNA vaccine A) is 3 or more doses of 25 μg -500 μg or 30 μg -450 μg. For example, the effective amount of hCMV immunogenic composition (e.g., mRNA vaccine A) may be 3 doses of 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg , 42 μg, 43 μg , 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg , 82 μg , 83 μg , 84 μg, 85 μg, 86 μg, 87 μg, 88 μg, 89 μg, 90 μg, 95 μg, 100 μg, 110 μg, 120 μg, 130 μg, 140 μg, 150 μg, 160 μg, 170 μg, 180 μg, 190 μg, 200 μg, 250 μg, 300 μg, 350 μg, 400 μg, 450 μg, or 500 μg, including all values in between.

In some embodiments, the effective amount of hCMV immunogenic composition (e.g., mRNA vaccine A) is 3 or more doses of 30 μg -180 μg. In some embodiments, the effective amount of hCMV immunogenic composition (e.g., mRNA vaccine A) is 3 or more doses of 30 pg. In some embodiments, the effective amount of hCMV immunogenic composition (e.g., mRNA vaccine A) is 3 or more doses of 90 μg. In some embodiments, the effective amount of hCMV immunogenic composition (e.g., mRNA vaccine A) is 3 or more doses of 180 μg. In some embodiments, the effective amount of hCMV immunogenic composition (e.g., mRNA vaccine A) is 3 or more doses of 300 μg -450 μg. In some embodiments, the effective amount of hCMV immunogenic composition (e.g., mRNA vaccine A) is 3 or more doses of 300 μg. In some embodiments, the effective amount of hCMV immunogenic composition (e.g., mRNA vaccine A) is 3 or more doses of 450 μg.

In some embodiments, the effective amount of the hCMV immunogenic composition (e.g., mRNA vaccine B, including mRNAs encoding pp65mut), as provided herein, may be around 30 μg, administered for example as a single dose or as three 10 μg doses. In some embodiments, the effective amount of hCMV immunogenic composition (e.g., mRNA vaccine B) is a single dose of 5 μg -100 μg or 10 μg -80 μg. For example, the effective amount of hCMV immunogenic composition (e.g., mRNA vaccine B) may be a single dose of 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, or 100 μg. In some embodiments, the effective amount of hCMV immunogenic composition (e.g., mRNA vaccine B) is a single dose of 10 μg-80 μg. In some embodiments, the effective amount of hCMV immunogenic composition (e.g., mRNA vaccine B) is a single dose of 10 μg. In some embodiments, the effective amount of hCMV immunogenic composition (e.g., mRNA vaccine B) is a single dose of 40 μg. In some embodiments, the effective amount of hCMV immunogenic composition (e.g., mRNA vaccine B) is a single dose of 80 μg.

In some embodiments, the effective amount of hCMV immunogenic composition (e.g., mRNA vaccine B) is 2 doses of 5 μg -100 μg or 10 μg-80 μg. For example, the effective amount of hCMV immunogenic composition (e.g., mRNA vaccine B) may be 2 doses of 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, or 100 μg. In some embodiments, the effective amount of hCMV immunogenic composition (e.g., mRNA vaccine B) is 2 doses of 10 μg-80 μg. In some embodiments, the effective amount of hCMV immunogenic composition (e.g., mRNA vaccine B) is 2 doses of 10 μg. In some embodiments, the effective amount of hCMV immunogenic composition (e.g., mRNA vaccine B) is 2 doses of 40 μg. In some embodiments, the effective amount of hCMV immunogenic composition (e.g., mRNA vaccine B) is 2 doses of 80 μg.

In some embodiments, the effective amount of hCMV immunogenic composition (e.g., mRNA vaccine B) is 3 or more doses of 5 -100 μg or 10 μg -80 μg. For example, the effective amount of hCMV immunogenic composition (e.g., mRNA vaccine B) may be 3 or more doses of 5 μg, 10 μg, 15 μg, 20 μg, 25 μg, 30 μg, 35 μg, 40 μg, 45 μg, 50 μg, 55 μg, 60 pg, 65 μg, 70 μg, 75 μg, 80 μg, 85 μg, 90 μg, 95 μg, or 100 μg. In some embodiments, the effective amount of hCMV immunogenic composition (e.g., mRNA vaccine B) is 3 or more doses of 10 μg-80 μg. In some embodiments, the effective amount of hCMV immunogenic composition (e.g., mRNA vaccine B) is 3 or more doses of 10 μg. In some embodiments, the effective amount of hCMV immunogenic composition (e.g., mRNA vaccine B) is 3 or more doses of 40 μg. In some embodiments, the effective amount of hCMV immunogenic composition (e.g., mRNA vaccine B) is 3 or more doses of 80 μg.

In some embodiments, one, two, three, or more than three doses (of any of the doses described herein) of the hCMV mRNA vaccine A and/or hCMV mRNA vaccine B are administered to a subject. In some embodiments, one, two, or three doses (of any of the doses described herein) of the hCMV mRNA vaccine A and hCMV mRNA vaccine B are administered to a subject. In some embodiments, the doses are administered on day 1, around the beginning of month 2 (e.g., day 29), and around the beginning of month 6 (e.g., day 169).

In some embodiments, a dose of hCMV mRNA vaccine A and/or hCMV mRNA vaccine B are administered to a subject on day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, day 31, day 32, day 33, day 34, day 35, day 36, day 37, day 38, day 39, day 40, day 41, day 42, day 43, day 44, day 45, day 46, day 47, day 48, day 49, day 50, day 51, day 52, day 53, day 54, day 55, day 56, day 57, day 58, day 59, day 60, day 61, day 62, day 63, day 64, day 65, day 66, day 67, day 68, day 69, day 70, day 71, day 72, day 73, day 74, day 75, day 76, day 77, day 78, day 79, day 80, day 81, day 82, day 83, day 84, day 85, day 86, day 87, day 88, day 89, day 90, day 91, day 92, day 93, day 94, day 95, day 96, day 97, day 98, day 99, day 100, day 101, day 102, day 103, day 104, day 105, day 106, day 107, day 108, day 109, day 110, day 111, day 112, day 113, day 114, day 115, day 116, day 117, day 118, day 119, day 120, day 121, day 122, day 123, day 124, day 125, day 126, day 127, day 128, day 129, day 130, day 131, day 132, day 133, day 134, day 135, day 136, day 137, day 138, day 139, day 140, day 141, day 142, day 143, day 144, day 145, day 146, day 147, day 148, day 149, day 150, day 151, day 152, day 153, day 154, day 155, day 156, day 157, day 158, day 159, day 160, day 161, day 162, day 163, day 164, day 165, day 166, day 167, day 168, day 169, day 170, day 171, day 172, day 173, day 174, day 175, day 176, day 177, day 178, day 179, day 180, day 181, day 182, day 183, day 184, day 185, day 186, day 187, day 188, day 189, day 190, day 191, day 192, day 193, day 194, day 195, day 196, day 197, day 198, day 199. In some embodiments, a dose of hCMV mRNA vaccine A and/or hCMV mRNA vaccine B are administered to a subject after day 199.

In some embodiments, the effective amount of an hCMV immunogenic composition (e.g., mRNA vaccine) is at least 1 dose (e.g., 1, 2, 3 doses at any of the dosages levels described herein, such as 30 μg, 90 μg, or 180 μg) of the hCMV immunogenic composition (e.g., mRNA vaccine). In some embodiments, the effective amount of an hCMV immunogenic composition (e.g., mRNA vaccine) is at least 1 dose (e.g., 1, 2, 3 doses at any of the dosages levels described herein, such as 10 μg, 40 μg, or 80 μg).

The hCMV immunogenic compositions (e.g., mRNA vaccines) described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the hCMV immunogenic composition (e.g., mRNA vaccine), wherein the hCMV immunogenic composition (e.g., mRNA vaccine) is formulated in an effective amount to produce an antigen specific immune response in a subject (e.g., production of antibodies specific to an anti-hCMV antigen). “An effective amount” is a dose of the hCMV immunogenic composition (e.g., mRNA vaccine) effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) hCMV protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+helper and/or CD8+T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function, and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+and CD8+T-cells.

In some embodiments, the antigen-specific immune response is characterized by measuring an anti-hCMV antigen antibody titer produced in a subject administered the hCMV mRNA vaccine as provided herein. An antibody titer is a measurement of the amount of antibodies within a subject, for example, antibodies that are specific to a particular antigen (e.g., an anti-hCMV antigen) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the hCMV mRNA vaccine.

In some embodiments, an anti-hCMV antigen antibody titer produced in a subject is increased by at least 1 log relative to a control. For example, anti-hCMV antigen antibody titer produced in a subject may be increased by at least 1.5, at least 2, at least 2.5, at least 3 log, at least 4 log, or at least 5 log , or more, relative to a control. In some embodiments, the anti-hCMV antigen antibody titer produced in the subject is increased by 1, 1.5, 2, 2.5, 3, 3.5, 4, 4.5, or 5 log relative to a control. In some embodiments, the anti-hCMV antigen antibody titer produced in the subject is increased by 1-5 log relative to a control. For example, the anti-hCMV antigen antibody titer produced in a subject may be increased by 1-1.5, 1-2, 1-2.5, 1-3, 1-4, 1-5, 1.5-2, 1.5-2.5, 1.5-3, 1.5-4, 1.5-5, 2-2.5, 2-3, 2-4, 2-5, 2.5-3, 2.5-4, 2.5-5, 3-4. 3-5, or 4-5 log relative to a control.

In some embodiments, the anti-hCMV antigen antibody titer produced in a subject is increased at least 2 times relative to a control. For example, the anti-hCMV antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-hCMV antigen antibody titer produced in the subject is increased 2, 3, 4, 5, 6, 7, 8, 9, or 10 times relative to a control. In some embodiments, the anti-hCMV antigen antibody titer produced in a subject is increased 2-10 times relative to a control. For example, the anti-hCMV antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to hCMV. A geometric mean titer (GMT) is the average antibody titer for a group of subjects calculated by multiplying all values and taking the nth root of the number, where n is the number of subjects with available data.

In some embodiments, administration of an effective amount of hCMV immunogenic composition (e.g., mRNA vaccine A) or an effective amount of hCMV immunogenic composition (e.g., mRNA vaccine B) elicits serum neutralizing antibody titers against hCMV. In some embodiments, administration a single dose (e.g., any of the doses described herein), or multiple doses, of hCMV mRNA vaccine A or a single dose (e.g., any of the doses described herein), or multiple doses, of hCMV mRNA vaccine B elicits serum neutralizing antibody titers against hCMV.

In some embodiments, the GMT of serum neutralizing antibodies to hCMV increases in the subject administered hCMV mRNA vaccine A by at least 3-fold or at least 4-fold, relative to baseline. For example, the GMT of serum neutralizing antibodies to hCMV may increase in the subject by at least 3-fold, at least 4-fold, at least 5-fold, at least 6-fold, at least 7-fold, at least 8-fold, at least 9-fold, or at least 10-fold, relative to baseline. In some embodiments, the serum neutralizing antibodies are antibodies against epithelial cell infection. In other embodiments, the serum neutralizing antibodies are antibodies against fibroblast infection. In some embodiments, the GMT of serum neutralizing antibodies to hCMV increases in the subject by 2-fold to 10-fold (e.g., at least 3-fold) after administering a single dose (e.g., a single dose of ≥30 μg, such as 30 μg, 90 μg, 180 μg, or 300 pig, or a single dose of 30-200 μg) of hCMV mRNA vaccine A, relative to baseline. In some embodiments, the serum neutralizing antibodies are antibodies against epithelial cell infection. In other embodiments, the serum neutralizing antibodies are antibodies against fibroblast infection.

In some embodiments, the GMT of serum neutralizing antibodies to hCMV increases in the subject by 2-fold to 10-fold (e.g., at least 3-fold) after administering two doses (e.g., two doses of ≥30 μg, such as 30 μg, 90 μg, 180 μg, or 300 μg, or two doses of 30-200 μg) of hCMV mRNA vaccine A, relative to baseline. In some embodiments, the GMT of serum neutralizing antibodies to hCMV increases in the subject by 2-fold to 10-fold after administering three doses (e.g., three doses of ≥30 μg, such as 30 μg, 90 μg, 180 or 300 μg, or three doses of 30-200 μg) of hCMV mRNA vaccine A, relative to baseline. In some embodiments, the serum neutralizing antibodies are antibodies against epithelial cell infection. In other embodiments, the serum neutralizing antibodies are antibodies against fibroblast infection.

In some embodiments, the GMT of serum neutralizing antibodies to hCMV increases in the subject administered hCMV mRNA vaccine A by 9-fold to 20-fold (e.g., 9-20, 10-20, 15-20, 9-15, 10-15, or 9-10 fold) after administering two doses (e.g., two doses of ≥30 μg, such as 30 μg, 90 μg, 180 μg, or 300 μg, or two doses of 30-200 μg) of hCMV mRNA vaccine A, relative to baseline. For example, the GMT of serum neutralizing antibodies to hCMV may increase in the subject by 9-fold, 10-fold, 11-fold, 12-fold, 13-fold, 14-fold, 15-fold, 16-fold, 17-fold, 18-fold, 19-fold, or 20-fold after administering two doses (e.g., two doses of ≥30 μg, such as 30 μg, 90 pz, 180 μg, or 300 μg, or two doses of 30-200 μg) of hCMV mRNA vaccine A, relative to baseline. In some embodiments, the serum neutralizing antibodies are antibodies against epithelial cell infection. In other embodiments, the serum neutralizing antibodies are antibodies against fibroblast infection.

In some embodiments, the GMT of serum neutralizing antibodies to hCMV increases in the subject administered hCMV mRNA vaccine A by up to 40-fold (e.g., up to 40, up to 35, up to 30, up to 25 fold) after administering three doses (e.g., three doses of ≥30 pig, such as 30 μg, 90 μg, 180 μg, or 300 μg, or three doses of 30-200 μg) of hCMV mRNA vaccine A, relative to baseline. For example, the GMT of serum neutralizing antibodies to hCMV may increase in the subject by 20-fold to 40-fold (e.g., 20-40, 20-35, 20-30, 20-25, 25-40, 25-35, 25-30, 30-40, 30-35, or 35-40 fold) after administering three doses (e.g., three doses of ≥30 μg, such as 30 μg, 90 μg, 180 μg, or 300 pig, or three doses of 30-200 μg) of hCMV mRNA vaccine A, relative to baseline. In some embodiments, the GMT of serum neutralizing antibodies to hCMV may increase in the subject by 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, or 40-fold after administering three doses (e.g., three doses of ≥30 μg, such as 30 μg, 90 μg, 180 μg, or 300 pig, or three doses of 30-200 μg) of hCMV mRNA vaccine A, relative to baseline. In some embodiments, the serum neutralizing antibodies are antibodies against epithelial cell infection. In other embodiments, the serum neutralizing antibodies are antibodies against fibroblast infection.

In some embodiments, the GMR for hCMV in subjects (e.g., seronegative subjects) administered at least one ≥30 μg dose (e.g., 1, 2, or 3 doses of 30 μg, 90 μg, or 180 μg, or 1, 2, or 3 doses of 30-200 μg) of hCMV mRNA vaccine A is in the range of 0.6-11. For example, the GMR for hCMV in subjects (e.g., seronegative subjects) administered at least one ≥30 μg dose (e.g., 1, 2, or 3 doses of 30 μg, 90 μg, or 180 μg, or 1, 2, or 3 doses of 30-200 μg) of hCMV mRNA vaccine A may be about 0.6, 0.8, 1, 2, 3, 4, 5, 6, 7, 8, 9, 10, or 11.

In some embodiments, the GMR for hCMV in subjects (e.g., seronegative subjects) administered at least one ≥30 μg dose (e.g., 1, 2, or 3 doses of 30 μg, 90 μg, or 180 μg, or 1, 2, or 3 doses of 30-200 μg) of hCMV mRNA vaccine A is in the range of 30-180. For example, the GMR for hCMV in subjects (e.g., seronegative subjects) administered at least one ≥30 μg dose (e.g., 1, 2, or 3 doses of 30 μg, 90 μg, or 180 μg, or 1, 2, or 3 doses of 30-200 μg) of hCMV mRNA vaccine A may be about 30, 35, 40, 45, 50, 55, 60, 65, 70, 75, 80, 85, 90, 95, 100, 110, 120, 130, 140, 150, 160, 170, or 180. In some embodiments, the average GMR for hCMV in subjects (e.g., seronegative subjects) administered at least one ≥30 .1,g dose (e.g., 1, 2, or 3 doses of 30 μg, 90 μg, or 180 μg doses, or 1, 2, or 3 doses of 30-200 μg) of hCMV mRNA vaccine A is about 120.

In some embodiments, the GMR for hCMV in subjects (e.g., seronegative subjects) administered at least one (e.g., 1 or 2) ≥30 μg dose of hCMV mRNA vaccine A is 30-40 (e.g., 31, 32, 33, 34, 35, 36, 37, 38, 39, or 40). In some embodiments, the GMR for hCMV in subjects (e.g., seronegative subjects) administered at least one (e.g., 1 or 2) ≥30 μg dose of hCMV mRNA vaccine A is about 38.15. In some embodiments, the GMR for hCMV in subjects (e.g., seronegative subjects) administered at least one (e.g., 1 or 2) ≥90 μg dose of hCMV mRNA vaccine A is 130-150 (e.g., 130, 135, 140, 145, or 150). In some embodiments, the GMR for hCMV in subjects (e.g., seronegative subjects) administered at least one (e.g., 1 or 2) ≥90 μg dose of hCMV mRNA vaccine A is about 142.57. In some embodiments, the GMR for hCMV in subjects (e.g., seronegative subjects) administered at least one (e.g., 1 or 2) ≥180 μg dose of hCMV mRNA vaccine A is 140-160 (e.g., 140, 145, 150, 155, or 160). In some embodiments, the GMR for hCMV in subjects (e.g., seronegative subjects) administered at least one (e.g., 1 or 2) ≥180 μg dose of hCMV mRNA vaccine A is about 157.96.

In some embodiments, the GMR for hCMV in subjects (e.g., seronegative subjects) administered at least one ≥30 μg dose (e.g., 1, 2, or 3 doses of 30 μg, 90 μg, 180 μg, or 300 μg, or 1, 2, or 3 doses of 30-200 μg) of hCMV mRNA vaccine A is in the range of 380-4000. For example, the GMR for hCMV in subjects (e.g., seronegative subjects) administered at least one ≥30 μg dose (e.g., 1, 2, or 3 doses of 30 90 μg, 180 μg, or 300 μg, or 1, 2, or 3 doses of 30-200 μg) of hCMV mRNA vaccine A may be about 380, 400, 450, 500, 550, 600, 650, 700, 750, 800, 850, 900, 1000, 1500, 2000, 2500, 3000, 3500, or 4000). In some embodiments, the average GMR for hCMV in subjects (e.g., seronegative subjects) administered at least one ≥30 μg dose (e.g., 1, 2, or 3 doses of 30 μg, 90 μg, 180 or 300 or 1, 2, or 3 doses of 30-200 μg) of hCMV mRNA vaccine A is about 2100.

In some embodiments, the GMR for hCMV in subjects (e.g., seronegative subjects) administered at least one (e.g., 1 or 2) ≥30 μg dose of hCMV mRNA vaccine A is 380-420 (e.g., 380, 390, 400, 410, or 420). In some embodiments, the GMR for hCMV in subjects (e.g., seronegative subjects) administered at least one (e.g., 1 or 2) ≥30 μg dose of hCMV mRNA vaccine A is about 407.86. In some embodiments, the GMR for hCMV in subjects (e.g., seronegative subjects) administered at least one (e.g., 1 or 2) ≥90 μg dose of hCMV mRNA vaccine A is 1800-2100 (e.g., 1800, 1900, 2000, or 2100). In some embodiments, the GMR for hCMV in subjects (e.g., seronegative subjects) administered at least one (e.g., 1 or 2) ≥90 μg dose of hCMV mRNA vaccine A is about 1913.17. In some embodiments, the GMR for hCMV in subjects (e.g., seronegative subjects) administered at least one (e.g., 1 or 2) ≥180 μg dose of hCMV mRNA vaccine A is 3600-4100 (e.g., 3600, 3700, 3800, 3900, 4000, or 4100). In some embodiments, the GMR for hCMV in subjects (e.g., seronegative subjects) administered at least one (e.g., 1 or 2) ≥180 μts dose of hCMV mRNA vaccine A is about 3842.87.

In some embodiments, the GMR for hCMV in subjects (e.g., seropositive subjects) administered at least one ≥30 μg dose (e.g., a single dose of 30 μg, 90 μg, 180 μg, or 300 μg, or a single dose of 30-200 μg) of hCMV mRNA vaccine A is in the range of 9-41. For example, the GMR for hCMV in subjects (e.g., seropositive subjects) administered at least one ≥30 μg dose (e.g., a single dose of 30 μg, 90 μg, 180 μg, or 300 μg, or a single dose of 30-200 μg) of hCMV mRNA vaccine A may be about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41. In some embodiments, the GMR is of neutralizing antibodies against epithelial cell infection.

In some embodiments, the GMR for hCMV in subjects (e.g., seropositive subjects) administered at least one ≥30 μg dose (e.g., a single dose of 30 μg, 90 μg, 180 μg, or 300 μg, or a single dose of 30-200 μg) of hCMV mRNA vaccine A is in the range of 4-8. For example, the GMR for hCMV in subjects (e.g., seropositive subjects) administered at least one ≥30 μg dose (e.g., a single dose of 30 μg, 90 μg, 180 μg, or 300 μg, or a single dose of 30-200 μg) dose of hCMV mRNA vaccine A may be about 4, 5, 6, 7, 8, 9, or 10. In some embodiments, the GMR is of neutralizing antibodies against fibroblast infection.

In some embodiments, the GMR for hCMV in subjects (e.g., seropositive subjects) administered at least one ≥30 μg dose (e.g., a single dose of 30 μg, 90 μg, 180 μg, or 300 μg, or a single dose of 30-200 μg) of hCMV mRNA vaccine A is in the range of 2-3. For example, the GMR for hCMV in subjects (e.g., seropositive subjects) administered at least one ≥30 μg dose (e.g., a single dose of 30 μg, 90 μg, 180 μg, or 300 μg, or a single dose of 30-200 μg) dose of hCMV mRNA vaccine A may be about 2, 2.1, 2.2, 2.3, 2.4, 2.5, 2.6, 2.7, 2.8, 2.9, or 3. In some embodiments, the average GMR for hCMV in subjects (e.g., seropositive subjects) administered at least one ≥30 gs dose (e.g., a single dose of 30 μg, 90 μg, 180 μs, or 300 μg, or a single dose of 30-200 μg) of hCMV mRNA vaccine A is about 2.7.

In some embodiments, the GMR for hCMV in subjects (e.g., seropositive subjects) administered a single dose of ≥30 μg of hCMV mRNA vaccine A is 2.3-2.5 (e.g., 2.3, 2.4, or 2.5). In some embodiments, the GMR for hCMV in subjects (e.g., seropositive subjects) administered a single dose of ≥30 μg of hCMV mRNA vaccine A is about 2.43. In some embodiments, the GMR for hCMV in subjects (e.g., seropositive subjects) administered a single dose of ≥90 μg of hCMV mRNA vaccine A is 2.5-2.8 (e.g., 2.5, 2.6, 2.7, or 2.8). In some embodiments, the GMR for hCMV in subjects (e.g., seropositive subjects) administered a single dose of ≥90 μg of hCMV mRNA vaccine A is about 2.66. In some embodiments, the GMR for hCMV in subjects (e.g., seropositive subjects) administered a single dose of ≥180 .1,g of hCMV mRNA vaccine A is 2.6-3 (e.g., 2.6, 2.7, 2.8, 2.9, or 3). In some embodiments, the GMR for hCMV in subjects (e.g., seropositive subjects) administered a single dose of ≥180 .1,g of hCMV mRNA vaccine A is about 2.83.

In some embodiments, the GMR for hCMV in subjects (e.g., seropositive subjects) administered at least one ≥30 μg dose (e.g., a single dose of 30 μg, 90 μg, 180 μg, or 300 μg, or a single dose of 30-200 μg) of hCMV mRNA vaccine A is in the range of 6-10. For example, the GMR for hCMV in subjects (e.g., seropositive subjects) administered at least one ≥30 μg dose (e.g., a single dose of 30 90 or 180 μg, or a single dose of 30-200 μg) dose of hCMV mRNA vaccine A may be about 6, 6.5, 7, 7.5, 8, 8.5, 9, 9.5, or 10. In some embodiments, the average GMR for hCMV in subjects (e.g., seropositive subjects) administered at least one ≥30 μg dose (e.g., a single dose of 30 μg, 90 μg, 180 μg, or 300 μg, or a single dose of 30-200 μg) of hCMV mRNA vaccine A is about 7.7.

In some embodiments, the GMR for hCMV in subjects (e.g., seropositive subjects) administered a single dose of ≥30 μg of hCMV mRNA vaccine A is 6-7 (e.g., 6.1, 6.2, 6.3, 6.4, 6.5, 6.6, 6.7, 6.8, 6.9, or 7). In some embodiments, the GMR for hCMV in subjects (e.g., seropositive subjects) administered a single dose of ≥30 μg of hCMV mRNA vaccine A is about 6.85. In some embodiments, the GMR for hCMV in subjects (e.g., seropositive subjects) administered a single dose of ≥90 μg of hCMV mRNA vaccine A is 6-7 (e.g., 6.1, 6.2, 6.3, 6.4, 6.5, 6.6, 6.7, 6.8, 6.9, or 7). In some embodiments, the GMR for hCMV in subjects (e.g., seropositive subjects) administered a single dose of ≥90 μg of hCMV mRNA vaccine A is about 6.93. In some embodiments, the GMR for hCMV in subjects (e.g., seropositive subjects) administered a single dose of ≥180 μg of hCMV mRNA vaccine A is 9-10 (e.g., 9.1, 9.2, 9.3, 9.4, 9.5, 9.6, 9.7, 9.8, 9.9, or 10). In some embodiments, the GMR for hCMV in subjects (e.g., seropositive subjects) administered a single dose of ≥180 μg of hCMV mRNA vaccine A is about 9.26.

In some embodiments, the GMR for hCMV in subjects (e.g., seropositive subjects) administered at least two ≥30 μg dose (e.g., a single dose of 30 μg, 90 μg, or 180 μg, or a single dose of 30-200 μg) of hCMV mRNA vaccine A is in the range of 2-5. For example, the GMR for hCMV in subjects (e.g., seropositive subjects) administered at least two ≥30 μg dose (e.g., a single dose of 30 μg, 90 μg, or 180 μg, or a single dose of 30-200 μg) of hCMV mRNA vaccine A may be about 2, 2.5, 3, 3.5, 4, 4.5, or 5. In some embodiments, the average GMR for hCMV in subjects (e.g., seropositive subjects) administered at least two ≥30 μg dose (e.g., a single dose of 30 μg, 90 μg, or 180 μg, or a single dose of 30-200 μg) of hCMV mRNA vaccine A is about 3.2.

In some embodiments, the GMR for hCMV in subjects (e.g., seropositive subjects) administered two doses of ≥30 .1,g of hCMV mRNA vaccine A is 12-14 (e.g., 12, 12.5, 13, 13.5, or 14). In some embodiments, the GMR for hCMV in subjects (e.g., seropositive subjects) administered two doses of ≥30 μg of hCMV mRNA vaccine A is about 13.15. In some embodiments, the GMR for hCMV in subjects (e.g., seropositive subjects) administered two doses of ≥90 μg of hCMV mRNA vaccine A is 8-10 (e.g.,8, 8.5, 9, 9.5, or 10). In some embodiments, the GMR for hCMV in subjects (e.g., seropositive subjects) administered two doses of ≥90 μg of hCMV mRNA vaccine A is about 9.91. In some embodiments, the GMR for hCMV in subjects (e.g., seropositive subjects) administered two doses of ≥180 μIs of hCMV mRNA vaccine A is 18-20 (e.g., 18, 18.5, 19, 19.5, or 20). In some embodiments, the GMR for hCMV in subjects (e.g., seropositive subjects) administered two doses of ≥180 μg of hCMV mRNA vaccine A is about 19.36.

In some embodiments, the GMR for hCMV in subjects (e.g., seropositive subjects) administered at least two ≥30 μg dose (e.g., at least two doses of 30 μg, 90 μg, 180 μIs, or 300 μg, or at least two doses of 30-200 μg) of hCMV mRNA vaccine A is in the range of 10-20. For example, the GMR for hCMV in subjects (e.g., seropositive subjects) administered at least two ≥30 μg dose (e.g., at least two doses of 30 μg, 90 μg, 180 μg, or 300 μg, or at least two doses of 30-200 μg) of hCMV mRNA vaccine A may be about 10, 11, 12, 13, 14, 15, 16, 17, 18, 19, or 20. In some embodiments, the average GMR for hCMV in subjects (e.g., seropositive subjects) administered at least two ≥30 μg dose (e.g., at least two doses of 30 μIs, 90 μg, 180 μg, or 300 μg, or at least two doses of 30-200 μg) of hCMV mRNA vaccine A is about 14.2.

In some embodiments, the GMR for hCMV in subjects (e.g., seropositive subjects) administered at least two ≥30 μg dose (e.g., at least two doses of 30 μg, 90 μg, 180 μg, or 300 μg, or at least two doses of 30-200 μg) of hCMV mRNA vaccine A is increased by at least 2.5-fold (e.g., at least 2.5-fold, at least 3-fold, at least 3.5 fold), relative to the baseline. For example, the GMR for hCMV in subjects (e.g., seropositive subjects) administered at least two ≥30 μg dose (e.g., at least two doses of 30 μg, 90 μg, 180 μIs, or 300 μIs, or at least two doses of 30-200 μg) of hCMV mRNA vaccine A may be may be increased by 2.5, 2.6, 2.7, 2.8, 2.9, 3, 3.1, 3.2, 3.3, 3.4, 3.5, 3.6, 3.7, or 3.8 fold relative to the baseline.

In some embodiments, the GMR for hCMV in subjects (e.g., seropositive subjects) administered at least three ≥30 μg dose (e.g., at least three doses of 30 μg, 90 μ,g, 180 μg, or 300 p,g, or at least three doses of 30-200 μg) of hCMV mRNA vaccine A is increased by at least 3.9-fold (e.g., at least 3.9-fold, at least 4-fold, at least 5 fold), relative to the baseline. For example, the GMR for hCMV in subjects (e.g., seropositive subjects) administered at least three ≥30 μg dose (e.g., at least three doses of 30 μg, 90 μg, 180 μg, or 300 Kg, or at least three doses of 30-200 μg) of hCMV mRNA vaccine A may be may be increased by 3.9, 4, 4.1, 4.2, 4.3, 4.4, 4.5, 4.6, 4.7, 4.8, 4.9, or 5-fold relative to the baseline.

A control/baseline, in some embodiments, is the anti-hCMV antigen antibody titer produced in a subject who has not been administered the hCMV mRNA vaccine. In some embodiments, a control/baseline is an anti-hCMV antigen antibody titer produced in a subject who has a natural hCMV infection, i.e., a subject who is hCMV seropositive prior to being administered the hCMV mRNA vaccine. In some embodiments, a control/baseline is an anti-hCMV antigen antibody titer produced in a subject who is hCMV seronegative prior to being administered the hCMV mRNA vaccine. In some embodiments, the GMT of serum neutralizing antibodies to hCMV increases in a dose-dependent manner.

In some embodiments, the ability of the hCMV immunogenic composition (e.g., mRNA vaccine) to be effective is measured in a murine model. For example, the hCMV immunogenic composition (e.g., mRNA vaccine) may be administered to a murine model and the murine model assayed for induction of neutralizing antibody titers. Viral challenge studies may also be used to assess the efficacy of a vaccine of the present disclosure. For example, the hCMV immunogenic composition (e.g., mRNA vaccine) may be administered to a murine model, the murine model challenged with hCMV, and the murine model assayed for survival and/or immune response (e.g., neutralizing antibody response, T cell response (e.g., cytokine response)).

In some embodiments, an effective amount of the hCMV immunogenic composition (e.g., mRNA vaccine) is a dose that is reduced compared to the standard of care dose of a recombinant hCMV protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified hCMV protein vaccine, or a live attenuated or inactivated hCMV mRNA vaccine, or a hCMV VLP vaccine, that a physician/clinician or other medical professional would administer to a subject to treat or prevent hCMV, or a hCMV-related condition, while following the standard of care guideline for treating or preventing hCMV, or a hCMV-related condition.

In some embodiments, the anti-hCMV antigen antibody titer produced in a subject administered an effective amount of the hCMV immunogenic composition (e.g., mRNA vaccine) is equivalent to an anti-hCMV antigen antibody titer produced in a control subject administered a standard of care dose of a recombinant or purified hCMV protein vaccine, or a live attenuated or inactivated hCMV mRNA vaccine, or a hCMV VLP vaccine.

Vaccine efficacy may be assessed using standard analyses (see, e.g., Weinberg et al., J Infect Dis. 2010 Jun. 1;201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun 1;201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of the hCMV mRNA vaccine is at least 60% relative to unvaccinated control subjects. For example, efficacy of the hCMV mRNA vaccine may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of an hCMV mRNA vaccine of the present disclosure is sufficient to provide sterilizing immunity in the subject for at least 1 year. For example, the effective amount of the hCMV mRNA vaccine of the present disclosure may be sufficient to provide sterilizing immunity in the subject for at least 2 years, at least 3 years, at least 4 years, or at least 5 years. In some embodiments, the effective amount of the hCMV mRNA vaccine of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of the hCMV mRNA vaccine of the present disclosure is sufficient to produce detectable levels of hCMV antigen as measured in serum of the subject at 1-72 hours post administration.

Titer. An antibody titer is a measurement of the amount of antibodies within a subject, for example, antibodies that are specific to a particular antigen (e.g., an anti-hCMV antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of the hCMV mRNA vaccine of the present disclosure is sufficient to produce a 1,000-10,000 neutralizing antibody titer produced by neutralizing antibody against the hCMV antigen as measured in serum of the subject at 1-72 hours post administration. In some embodiments, the effective amount is sufficient to produce a 1,000-5,000 neutralizing antibody titer produced by neutralizing antibody against the hCMV antigen as measured in serum of the subject at 1-72 hours post administration. In some embodiments, the effective amount is sufficient to produce a 5,000-10,000 neutralizing antibody titer produced by neutralizing antibody against the hCMV antigen as measured in serum of the subject at 1-72 hours post administration.

In some embodiments, the neutralizing antibody titer is at least 100 NT50. For example, the neutralizing antibody titer may be at least 200, 300, 400, 500, 600, 700, 800, 900 or 1000 NT₅₀. In some embodiments, the neutralizing antibody titer is at least 10,000 N₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-hCMV antigen antibody titer produced in the subject is increased by at least 1 log relative to a control. For example, an anti-hCMV antigen antibody titer produced in the subject may be increased by at least 2, 3, 4, 5, 6, 7, 8, 9 or 10 log relative to a control.

In some embodiments, an anti-hCMV antigen antibody titer produced in the subject is increased at least 2 times relative to a control. For example, an anti-hCMV antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

### EXAMPLES

In order that the invention described in this application may be more fully understood, the following examples are set forth. The examples described in this application are offered to illustrate the systems and methods provided in this application and are not to be construed in any way as limiting their scope.

### Example 1.

### A Phase I, Randomized, Observer-Blind, Placebo-Controlled, Dose-Ranging Study to Evaluate the Safety, Reactogenicity, and Immunogenicity of Cytomegalovirus Vaccines hCMV mRNA Vaccine A and hCMV mRNA Vaccine B when Administered to Healthy Adults

The purpose of this Phase I study was to assess the safety, reactogenicity, and immunogenicity of hCMV mRNA vaccine A and the safety and reactogenicity of hCMV mRNA vaccine B.

hCMV mRNA vaccine A consists of distinct mRNA molecules having sequences that encode the full length CMV glycoprotein B (gB) and the pentameric gH/gL/UL128/UL130/UL131A glycoprotein complex (Pentamer). hCMV mRNA vaccine B consists of an mRNA molecule having a sequence that encodes a phosphorylation mutant of the pp65 protein, which lacks amino acids 435-438, and is aimed at eliciting T-cell responses. The phosphorylation site has been deleted in order to mitigate any theoretical safety concerns of expressing wild type pp65 protein.

Both hCMV mRNA vaccine A and hCMV mRNA vaccine B have demonstrated non-clinical safety and immunogenicity and thus hold the potential for preventing human primary CMV infection and CMV re-infection/re-activation in CMV-positive individuals.

### Choice of Study Population

Immune response to investigational CMV vaccines in subjects who are seronegative may be different from responses in those who are seropositive. To explore the potential differences in safety and immunogenicity, this study enrolled approximately equal numbers of healthy CMV-seronegative and CMV-seropositive subjects in dose-selection phase B and the expansion cohorts of sentinel-expansion phase C of the study.

### Regimen and Dose

Five dose levels of hCMV mRNA vaccine A (30 μg, 90 μg, 180 μg, 300 μg, and either 240 μg or 450 μg [depending on safety review of Phase C Arm 1]) and 3 dose levels of hCMV mRNA vaccine B (10 μg, 40 μg, and 80 μg) are tested in this Phase I study.

The initial doses for both compounds are within the range for which non-clinical safety and immunogenicity data have been evaluated. There were no toxic effects, but some mild and expected local inflammatory reactions were observed. The dose levels up to 180 μg are also within the dose range that had a favorable safety profile and induced immune responses in a Phase 1/2 mRNA vaccine study (Bahl et al 2017).

Previous experience with other investigational CMV vaccines indicated the need for a 2-dose or 3-dose vaccination series to induce a robust and durable immune response (Pass et al 2009; Bernstein et al 2016). Thus, a 3-dose vaccination series was evaluated in this clinical study.

The planned dose schedule of administration (Day 1, Month 2, and Month 6) had previously been found to be the optimal vaccination schedule for recombinant protein vaccines. This schedule also allowed co-administration of the vaccine in the target population with human papillomavirus (HPV; Gardasil 2015) or hepatitis B virus (HBV; Engerix-B 2016) vaccines. The immune response was evaluated after each study vaccination as well as 4 months after the second dose and 6 months after the third dose.

### Safety Monitoring Plan

As hCMV mRNA vaccine A and hCMV mRNA vaccine B was administered for the first time to humans, safety precautions such as sequential enrollment, dose escalation, and continuous safety evaluations are taken. Study vaccines were initially administered to a small number of subjects and then, following the confirmation of acceptable tolerability, enrollment is expanded. Study pause rules were defined, and safety evaluation from this study are overseen by an Internal Safety Team (IST) and an unblinded, independent Safety Monitoring Committee (SMC). The study is conducted in multiple phases as described below.

### Use of Placebo

Because there are currently no licensed CMV vaccines available, a placebo group was used as a control for the safety, reactogenicity, and immunogenicity assessments.

### Study Blinding

Because the physical appearance of the placebo was different from hCMV mRNA vaccine A and hCMV mRNA vaccine B, and because hCMV mRNA vaccine A and hCMV mRNA vaccine B required dilution prior to administration, the study was conducted in an observer-blinded manner. In order to minimize bias, doses were administered by unblinded medical personnel in a manner that shielded both the subject and blinded site personnel from viewing the dose. The unblinded medical personnel did not participate in any per-protocol clinical evaluations.

### Risk/Benefit Assessment

Approximately 170 subjects in this study were exposed to hCMV mRNA vaccine A or hCMV mRNA vaccine B, and approximately 46 subjects received placebo.

### Study Objectives

### Primary Objective

The primary objective of this study was to evaluate the safety and reactogenicity of different dose levels of hCMV mRNA vaccine A and hCMV mRNA vaccine B, administered according to a 3-dose vaccination schedule.

### Secondary Objectives

Secondary objectives of the study were the following:
1. To evaluate neutralizing anti-CMV antibody responses against epithelial cell and fibroblast cell infection following vaccination with different doses of hCMV mRNA vaccine A.
2. To evaluate antigen-specific antibody responses against gB and Pentamer as measured by enzyme-linked immunosorbent assay (ELISA) following vaccination with different doses of hCMV mRNA vaccine A.
3. To compare the antibody-mediated immune responses elicited by different doses of hCMV mRNA vaccine A after the second and third vaccinations to inform selection of the optimal dose level of this vaccine for further evaluation.
4. To compare the antibody-mediated immune responses elicited by different doses of hCMV mRNA vaccine A at 4 months after the second vaccination and at 6 months after the third vaccination.
5. To evaluate the immunogenicity of hCMV mRNA vaccine A by baseline CMV serostatus.

### Exploratory Objectives

The exploratory objectives of the study were the following:
1. To explore the quality of antibody response following vaccination with different doses of hCMV mRNA vaccine A.
2. To evaluate antigen-specific T-cell responses to different doses of hCMV mRNA vaccine A as determined by enzyme-linked immunospot (ELISPOT) assay.
3. To assess antigen-specific memory B-cell responses to different doses of hCMV mRNA vaccine A, as detected by ELISPOT assay.
4. To assess gB-specific and Pentamer-specific plasmablasts following vaccination with different doses of hCMV mRNA vaccine A, as measured by flow cytometry.
5. To evaluate antigen-specific T cell responses to different doses of hCMV mRNA vaccine A as determined by intracellular cytokine staining analysis.
6. To assess the anti-gB and anti-Pentamer avidity index of different doses of hCMV mRNA vaccine A, as measured by ELISA.

### Study Methodology

### Experimental Design

This was a randomized observer-blind, placebo-controlled, dose-ranging, first-in-human study to evaluate the safety, reactogenicity, and immunogenicity of hCMV mRNA vaccine A and the safety and reactogenicity of hCMV mRNA vaccine B administered to healthy adults.

### Duration of the Study

The study duration was approximately 18 months for each subject.

### Phases of the Study

As hCMV mRNA vaccine A and hCMV mRNA vaccine B was administered for the first time to humans in this study, safety precautions were taken by utilizing enrollment into dose-escalation and dose-selection phases for the 3 lower dose levels and enrollment into sentinel-expansion cohorts for the other 2 dose levels.

Dose-escalation phase A: Sequential enrollment of 27 CMV-seronegative subjects into the 3 lower dose levels of the study vaccines or placebo. Nine subjects per dose level were randomly assigned in a 4:4:1 ratio to receive hCMV mRNA vaccine A (30, 90, or 180 μg), hCMV mRNA vaccine B (10, 40, or 80 μg), or placebo. Safety reviews by the Internal Safety Team (IST) permitted dose continuation within each dose level and dose escalation to the next dose level. The Safety Monitoring Committee (SMC) reviewed all safety and reactogenicity data through Day 63 (6 days after the second vaccination in the 180 μg/80 μg dose level) for hCMV mRNA vaccine A and hCMV mRNA vaccine B and confirmed the hCMV mRNA vaccine A dose levels evaluated in dose-selection phase B of the study, pending SMC safety review of dose-escalation phase B through Day 63. The SMC also reviews all available safety data through Day 175 (6 days after the third vaccination in the 180 μg/80 μg dose level) for hCMV mRNA vaccine A and hCMV mRNA vaccine B to permit administration of the third vaccination of hCMV mRNA vaccine A in dose-selection phase B.

Dose-escalation phase B: To implement hCMV mRNA vaccine A in dose-selection phase B, 15 CMV-seronegative subjects were enrolled sequentially into the 3 lower dose levels of hCMV mRNA vaccine A or placebo. Five subjects per dose level were randomly assigned in a 4:1 ratio to receive hCMV mRNA vaccine A or placebo. Safety reviews by the IST permit dose continuation within each dose level and escalation to the next dose level. Following review of all safety and reactogenicity data of all dose levels through Day 63 (6 days after the second vaccination in the 180 μg dose level) for hCMV mRNA vaccine A, the SMC confirms the hCMV mRNA vaccine A dose levels evaluated in dose-selection phase B of the study. The SMC also reviews all available safety data through Day 175 (6 days after the third vaccination of the 180 μg dose level) for hCMV mRNA vaccine A to permit administration of the third vaccination in dose-selection phase B.

Dose-selection phase B: Parallel enrollment of approximately 104 subjects (26 per study group) into the 3 lower dose levels of hCMV mRNA vaccine A or placebo. Subjects were randomly assigned in a 1:1:1:1 ratio to receive 30, 90, or 180 μg hCMV mRNA vaccine A or placebo. Approximately equal numbers of CMV-seronegative and CMV-seropositive subjects were enrolled at each dose level. Safety and reactogenicity are periodically reviewed by the unblinded SMC.

Sentinel-expansion phase C: To better understand the relationship between dose, tolerability, and immunogenicity, this phase will enroll up to 70 subjects (up to 2 arms, 35 subjects per arm) into 2 other dose levels of hCMV mRNA vaccine A or placebo. For each arm, enrollment is split into a sentinel cohort (5 CMV-seronegative subjects randomly assigned in a 4:1 ratio received hCMV mRNA vaccine A or placebo) and an expansion cohort (up to 30 subjects randomly assigned in a 4:1 ratio received hCMV mRNA vaccine A or placebo), with approximately equal numbers of CMV-seronegative and CMV-seropositive subjects.

Arm 1: Sentinel subjects are randomly assigned to receive 300 μg of hCMV mRNA vaccine A or placebo, based on safety data from dose-escalation phase B through Day 63 (6 days after the second vaccination). The SMC reviews all safety and reactogenicity data from the Arm 1 sentinel cohort through Day 7 (6 days after the first vaccination) to permit enrollment of the Arm 1 expansion cohort. The SMC then reviews safety and reactogenicity data from all Arm 1 subjects through Day 7 to permit enrollment into Arm 2.

Arm 2: Subjects are randomly assigned to receive hCMV mRNA vaccine A or placebo based on safety and tolerability data from Arm 1 through Day 7 (6 days after the first vaccination). The dose level of hCMV mRNA vaccine A in Arm 2 are determined in the following manner:

When SMC review of all Arm 1 subjects through Day 7 raises no safety concerns, Arm 2 subjects are randomly assigned to receive a dose level of 450 μg of hCMV mRNA vaccine A or placebo.

For safety concerns that arose during the enrollment of Arm 1 or after SMC review of all Arm 1 subjects through Day 7, Arm 2 subjects are randomly assigned to receive a dose level of 240 μg of hCMV mRNA vaccine A or placebo.

The IST reviews all safety and reactogenicity data from the Arm 2 sentinel cohort through Day 7 (6 days after the first vaccination) to permit enrollment of the Arm 2 expansion cohort.

Vaccination schedule: Three injections, given at Day 1, Month 2, and Month 6 (1 month=28 days)
Control: Saline placebo
Study groups:

### Dose-Escalation Phase A (CMV-Seronegative Subjects):

9 subjects randomly assigned in a 4:4:1 ratio received 30 μg hCMV mRNA vaccine A, 10 μg hCMV mRNA vaccine B, or placebo
9 subjects randomly assigned in a 4:4:1 ratio received 90 μg hCMV mRNA vaccine A, 40 μg hCMV mRNA vaccine B, or placebo
9 subjects randomly assigned in a 4:4:1 ratio received 180 μg hCMV mRNA vaccine A, 80 μg hCMV mRNA vaccine B, or placebo

### Dose-Escalation Phase B (CMV-Seronegative Subjects):

30 μg dose level: 5 subjects randomly assigned in a 4:1 ratio receive 30 μg hCMV mRNA vaccine A or placebo
90 μg dose level: 5 subjects randomly assigned in a 4:1 ratio receive 90 μg hCMV mRNA vaccine A or placebo
180 μg dose level: 5 subjects randomly assigned in a 4:1 ratio receive 180 μg hCMV mRNA vaccine A or placebo

### Dose-Selection Phase B:

104 subjects randomly assigned in a 1:1:1:1 ratio with approximately equal numbers of CMV-seropositive and CMV-seronegative subjects enrolled at each dose level.
Approximately 26 subjects received 30 μg hCMV mRNA vaccine A
Approximately 26 subjects received 90 μg hCMV mRNA vaccine A
Approximately 26 subjects received 180 μg hCMV mRNA vaccine A
Approximately 26 subjects received placebo

The hCMV mRNA vaccine A dose levels evaluated in dose-selection phase B of the study were confirmed by the SMC following review of the safety and reactogenicity data from dose-escalation phase B.

### Sentinel-Expansion Phase C:

Up to 70 subjects (up to 2 arms, 35 subjects per arm) are enrolled in a sentinel-expansion manner, randomly assigned in a 4:1 ratio to receive 2 other dose levels of hCMV mRNA vaccine A or placebo. Sentinel cohorts enroll CMV-seronegative subjects and expansion cohorts enroll approximately equal numbers of CMV-seronegative and CMV-seropositive subjects.
Arm 1: Approximately 35 subjects randomly assigned in a 4:1 ratio receive 300 μg of hCMV mRNA vaccine A or placebo.
Arm 2: Approximately 35 subjects randomly assigned in a 4:1 ratio receive either 240 or 450 μg hCMV mRNA vaccine A (depending on safety review of Phase C Arm 1) or placebo. The dose level for Arm 2 is based on safety data from all Arm 1 subjects through Day 7.

### Randomization

An Interactive Response Technology was used in the study. The number of arms and the randomization ratio for dose-selection phase B was adjusted according to SMC recommendations based on the review of the safety and reactogenicity data from the dose-escalation phases.

### Blinding

This was an observer-blind study.

### Blood Sample Schedule

Blood samples for screening laboratory testing were collected at the Screening visit. Blood samples for safety laboratory assessments are collected at Visits Day 1, Day 7, Month 1, Month 2, Day 63, Month 3, Month 6, Day 175, and Month 7. Blood samples for antibody-mediated immunogenicity are collected at Visits Day 1, Month 1, Month 3, Month 6, Month 7, and Month 12. Blood samples for assessment of cell-mediated immunogenicity are collected from subjects in dose-escalation phase B, dose-selection phase B, and sentinel-expansion phase C at Visits Day 1, Day 7, Month 2, Day 63, Month 6, Day 175, and Month 12.

Data Collection: Electronic Case Report Forms (eCRFs).

### Study Visits

Eleven clinic visits at Screening, Day 1, Day 7, Month 1, Month 2, Day 63, Month 3, Month 6, Day 175, Month 7, and Month 12.

Safety phone calls

Six safety phone calls are conducted approximately 24 and 48 hours after each study vaccination (Days 2, 3, 58, 59, 170, and 171) in all subjects in dose-escalation phases A and B and sentinel cohorts of sentinel-expansion phase C to collect solicited adverse events (AEs) and other safety information. In addition, 7 safety phone calls (Months 4 and 5, Months 8-11, and Month 18) are made to all subjects in the study to collect any medically-attended adverse effects (AEs), AEs leading to study withdrawal, serious AEs (SAEs), AEs of special interest (AESIs), information on concomitant medications associated with those events, and any vaccinations.

### Solicited AEs

Local (injection site pain, erythema, and swelling) and systemic (headache, fatigue, myalgia [muscle aches all over the body], arthralgia [aching in several joints], nausea, rash, fever, and chills) solicited AEs that occur from the time of each study vaccination through the following 6 days are recorded daily using Diary Cards for all subjects.

### Unsolicited AEs

All observed or reported AEs that occurred through 28 days after each study vaccination and were not included as part of the protocol-defined solicited AEs are recorded using Diary Cards for all subjects. In addition, qualified site personnel interview the subject during the site visit approximately 28 days after each vaccination (Visits Month 1, Month 3, and Month 7) to assess the occurrence of any unsolicited AEs.

### Medically-Attended AEs, AEs Leading to Study Withdrawal, AESIs and SAEs

Medically-attended AEs and AEs leading to study withdrawal, are collected from Day 1 and AESIs and SAEs are collected from the time the informed consent form is signed. These data are captured through the Diary Card, by interviewing subjects during site visits and safety phone calls, and by reviewing available medical records.

### Written Informed Consent

Written informed consent was obtained before conducting any study-specific procedures.

### Number of Subjects Planned

A total of approximately 216 subjects are enrolled into this study: 27 subjects in dose-escalation phase A, 15 subjects in dose-escalation phase B, 104 subjects in dose-selection phase B, and up to 70 subjects in sentinel-expansion phase C. Upon completion of all screening procedures, the Investigator reviews the inclusion/exclusion criteria for each subject to determine if the subject is eligible to enroll in the study. Their screening information is recorded on the appropriate eCRF page.

If the Investigator believes there is a reason to do so, some screening procedures are repeated once, with the exception of the laboratory parameters specified below.

Rescreening of an eligible subject is allowed if their originally intended dose level closes and their 28 day screening window was surpassed before another dose level opens. The subject is assigned a new screening number and all screening procedures are repeated. Subjects who did not meet all enrollment criteria at their first screening are not allowed to rescreen.

Screen failures were defined as subjects who signed the consent form but who were not subsequently randomly assigned to the study intervention or entered in the study. Information on eligibility, demographics, SAEs, and informed consent was collected for all screen failures.

### Subject Characteristics and Main Criteria for Inclusion and Exclusion

Subjects were included in the study if in good health as judged by physical examination and medical history and if they meet all specified eligibility criteria. Inclusion and exclusion criteria are provided below.

### Inclusion Criteria

Adult subjects (18 through 49 years of age) who were determined to be in good health, at the time of first vaccination, in the opinion of the Investigator, as determined by medical history, clinical laboratory assessments, vital sign measurements, and physical examination findings at Screening. Subjects who were available for all study visits, and had given written informed consent. Subjects who had a body mass index (BMI) from 18 through 35 kg/m². Subjects who understood and agreed to comply with the study procedures and provided written informed consent. In the opinion of the Investigator, could and would comply with the requirements of the protocol (e.g., complete Diary Cards, return for follow-up visits, be available for safety phone calls).

Female subjects of non-childbearing potential may be enrolled in the study. Non-childbearing potential is defined as bilateral tubal ligation >1 year prior to Screening, bilateral oophorectomy, or hysterectomy or menopause (refer to the Glossary of Terms). A follicle stimulating hormone level may be measured at the discretion of the Investigator to confirm menopausal status. Female subjects of childbearing potential must have a negative pregnancy test at Screening and the day of vaccination and must have practiced adequate contraception or abstaining from all activities which could lead to pregnancy for 30 days prior to the first vaccination, and must have agreed to continue adequate contraception through 3 months following the last vaccination. Male subjects must agree to practice adequate contraception for 30 days prior to the first vaccination and through 3 months following the last vaccination.

### Exclusion Criteria

Any acute or chronic disease determined to be clinically significant by the Investigator, including an immune-mediated disease or immunosuppressive condition. Asymptomatic conditions or findings (e.g., mild hypertension, dyslipidemia) were not exclusionary if they were being appropriately managed, are clinically stable, and were unlikely to progress within the study period, in the opinion of the Investigator. Has a diagnosis of malignancy within the previous 10 years (excluding nonmelanoma skin cancer). If female and of childbearing potential, was pregnant or lactating, had not adhered to an adequate contraception method from at least 30 days before study entry, or did not plan to do so for at least 3 months after the last vaccination. If female and of childbearing potential, was pregnant or lactating, had not adhered to an adequate contraception method from at least 30 days before study entry, or did not plan to do so for at least 3 months after the last vaccination. Abnormal screen blood tests including elevated liver function tests, defined as aspartate aminotransferase, alanine aminotransferase, or alkaline phosphatase, or elevated creatinine or reduced platelets, with a toxicity score of Grade ≥1 at Screening. Retesting of these parameters is not allowed. Has safety laboratory test results (hematology, chemistry, and coagulation) with a toxicity score of Grade ≥2 at Screening. The inclusion of subjects with non clinically significant (NCS) Grade 1 laboratory abnormalities was allowed based on the Investigator's discretion. Had been administered any investigational or non-registered product (drug or vaccine) other than the study vaccine within 30 days preceding the first dose of study vaccine or had plans for administration during the study period. Previously participated in an investigational study involving lipid nanoparticles (LNPs). Had a positive test result at Screening for hepatitis B surface antigen, hepatitis C virus antibody, or human immunodeficiency virus type 1 or 2 antibodies. At Screening, had a positive urine drug screen for any of the following nonprescription drugs of abuse: amphetamines, benzodiazepines, cocaine, methadone, opiates, and phencyclidine. A positive test result for any other drug required Investigator approval prior to inclusion of the subject. Positive urine drug screens for amphetamines, benzodiazepines, or opiates are not exclusionary if the positive result is due to a prescribed concomitant medication, in the opinion of the Investigator. Had chronic administration (defined as more than 14 days within 3 months before the first vaccination) of potentially hepatotoxic drugs or have other medical conditions that affect the liver (e.g., alcohol abuse). Had a history of idiopathic urticaria. Had plans for administration or had been administered a vaccine not foreseen by the study protocol within the period from 30 days before through 30 days after each study vaccination, except for any licensed influenza vaccine administered ≥15 days before or after any study vaccination. Had chronic administration (defined as more than 14 days in total) of immunosuppressants or other immune-modifying drugs within 6 months before the first vaccine dose (for corticosteroids: prednisone ≥20 mg/day or equivalent is not permitted). Inhaled, nasal, and topical steroids are allowed. Was administered immunoglobulins and/or blood products within the 3 months before the first study vaccine dose or had plans for administration during the study period. Had a history of hypersensitivity or severe reactions to previous vaccinations (e.g., anaphylaxis, urticaria, other significant reaction requiring medical intervention). Had a bleeding disorder that is considered a contraindication to IM injection or phlebotomy. Was acutely ill or febrile on presentation to the Screening visit. Fever is defined as a temperature ≥38.0° C/100.4° F. by the oral, axillary, or tympanic route. Subjects meeting this criterion may be rescheduled for Screening at a later date. Afebrile subjects with minor illnesses can be enrolled at the discretion of the Investigator. Any medical, psychiatric, or occupational condition that, in the opinion of the Investigator, might pose an additional risk to the subject due to participation in the study or can interfere with the evaluation of the study vaccines or the interpretation of study results. Subjects who were seropositive for CMV at Screening are excluded from dose-escalation phases A and B and sentinel cohorts of sentinel-expansion phase C. Was an immediate family member or household member of study personnel. Had donated greater than 450 mL of whole blood or blood products within 30 days of dosing. Reported a history of a seizure disorder for which anticonvulsants are currently prescribed. The following were not reasons for exclusion: a reported history of seizure disorder but no seizures and no prescribed anticonvulsants within the last 5 years and febrile seizures during childhood.

### Study Vaccine

hCMV mRNA vaccine A consists of 6 mRNA Drug Substances in a liquid nanoparticle (LNP) formulation. hCMV mRNA vaccine B consists of a single mRNA Drug Substance in an LNP formulation.

The LNP formulation for each vaccine includes 4 lipid excipients: an ionizable amino lipid, and the commercially-available lipids cholesterol, 1,2-distearoyl-sn-glycero-3-phosphocholine, and 1,2-dimyristoyl-sn-glycerol, methoxypolyethyleneglycol.

hCMV mRNA vaccine A and hCMV mRNA vaccine B are each provided as a sterile liquid for injection at concentrations of 1.0 and 2.0 mg/mL, respectively, in 93 mM Tris buffer, 7% propylene glycol, and 1 mM DTPA. The placebo is 0.9% sodium chloride.

Study vaccines were administered intramuscularly into the deltoid muscle, preferably in the non-dominant arm.

### Criteria for Evaluation

### Primary Safety and Reactogenicity Endpoints:

1. Occurrence of each solicited local and systemic AE during a 7-day follow-up period after each vaccination (i.e., the day of vaccination and 6 subsequent days).
2. Occurrence of any unsolicited AE during a 29-day follow-up period after each study vaccination (i.e., the day of vaccination and 28 subsequent days).
3. Occurrence of any laboratory test abnormality at Visits Day 1, Day 7, Month 1, Month 2, Day 63, Month 3, Month 6, Day 175, and Month 7.
4. Occurrence of any medically-attended AE from Day 1 to Month 18.
5. Occurrence of any AESI from Day 1 to Month 18.
6. Occurrence of any SAE from Day 1 to Month 18.

### Secondary Immunogenicity Endpoints:

1. Geometric mean titer (GMT) of neutralizing anti-CMV antibodies against epithelial cell infection on Visits Day 1, Month 1, Month 3, Month 6, Month 7, and Month 12.
2. Geometric mean titer of neutralizing anti-CMV antibodies against fibroblast cell infection on Visits Day 1, Month 1, Month 3, Month 6, Month 7, and Month 12.
3. Proportion of subjects with a ≥2-fold, 3-fold, and 4-fold increase in serum neutralizing anti-CMV antibodies against epithelial cell infection on Visits Month 1, Month 3, Month 6, Month 7, and Month 12 compared with Day 1.
4. Proportion of subjects with a ≥2-fold, 3-fold, and 4-fold increase in serum neutralizing anti-CMV antibodies against fibroblast cell infection on Visits Month 1, Month 3, Month 6, Month 7, and Month 12 compared with Day 1.
5. Proportion of subjects with serum neutralizing anti-CMV antibody titer against epithelial cell infection above cut-off level (i.e., neutralizing antibody titers associated with natural CMV infection) at Visits Day 1, Month 1, Month 3, Month 6, Month 7, and Month 12.
6. Proportion of subjects with serum neutralizing anti-CMV antibody titer against fibroblast cell infection above cut-off level (i.e., neutralizing antibody titers associated with natural CMV infection) at Visits Day 1, Month 1, Month 3, Month 6, Month 7, and Month 12.
7. Reverse cumulative distribution of serum neutralizing antibody titers against epithelial and fibroblast cell infection on Visits Day 1, Month 1, Month 3, Month 6, Month 7, and Month 12.
8. Geometric mean titer of anti-gB immunoglobulin G (IgG) as measured by ELISA on Visits Day 1, Month 1, Month 3, Month 6, Month 7, and Month 12.
9. Geometric mean titer of anti-Pentamer IgG as measured by ELISA on Visits Day 1, Month 1, Month 3, Month 6, Month 7, and Month 12.

### Exploratory Immunogenicity Endpoints

1. Anti-gB and anti-Pentamer avidity index as measured by ELISA on Visits Day 1, Month 1, Month 3, Month 6, Month 7, and Month 12.
2. Frequencies of gB-specific and Pentamer-specific CD4 and CD8 T cells secreting IFN-γ as determined by ELISPOT on Visits Day 1, Day 7, Month 2, Day 63, Month 6, Day 175, and Month 12.
3. gB-specific and Pentamer-specific IgG memory B-cell responses as measured by ELISPOT on Visits Day 1, Day 7, Month 2, Day 63, Month 6, Day 175, and Month 12.
4. gB-specific and Pentamer-specific plasmablasts as measured by flow cytometry on Day 1, Day 7, Month 2, Day 63, Month 6, Day 175, and Month 12.
5. Frequencies of gB-specific and Pentamer-specific CD4 and CD8 T cells secreting IFN-γ, IL-2, and/or TNF-a as determined by intracellular cytokine staining on Visits Day 1, Day 7, Month 2, Day 63, Month 6, Day 175, and Month 12.

### Sample Size Justification:

Sample size was not driven by statistical assumptions for formal hypothesis testing, as previous clinical study data are not available and clinically meaningful group differences have not been established. In addition, values for a correlate of protection are not known at present; however, some data from the neutralization assay for human serum are available. The number of proposed subjects was sufficient to provide a descriptive summary of the safety and immunogenicity of hCMV mRNA vaccine A and a descriptive summary of the safety of hCMV mRNA vaccine B in dose-escalation phase A.

Anticipating that approximately 10% of the subjects will be non-evaluable (i.e., lost to follow-up, insufficient samples, or incomplete laboratory test results, etc.), approximately 216 subjects are enrolled to obtain approximately 194 evaluable subjects. Approximately equal numbers of CMV-seropositive and CMV-seronegative subjects are enrolled in dose-selection phase B and the expansion cohorts of sentinel-expansion phase C.

### Statistical Analyses:

Values below the lower limit of quantitation (LLOQ) for each assay are set at 50% of LLOQ. GMT antibody titers are logarithmically transformed (base 10). For each study group, GMTs of the serum neutralizing antibodies along with their associated 95% CIs are computed by exponentiation of the corresponding log-transformed means and 95% CIs.

Fold-rise: Proportions of subjects with a ≥2-fold, 3-fold, and 4-fold increase in serum neutralizing antibody titer (ELISA antibody concentration) against the respective vaccine antigen from baseline are assessed, for each study group, together with their two-sided 95% Clopper-Pearson CIs.

The statistical analyses for GMTs is conducted using an analysis of covariance model with dose level as fixed effects and baseline antibody level as covariates.

The primary immunogenicity analyses are based on a Per-protocol Set. If the number of subjects in the Full Analysis Set (FAS) and Per-protocol Set differ (defined as the difference divided by the total number of subjects in the PP set) by more than 10%, primary immunogenicity analyses will also be conducted on the FAS.

Planned analyses: Statistical analyses are performed as follows:
1. A 3-month interim analysis of safety, reactogenicity, and immunogenicity data collected from Visit Day 1 through Month 3 may be performed for each study phase. This analysis may be performed separately for Arms 1 and 2 of phase C. The analyses are conducted on cleaned data and are reported on a treatment group level. Available safety and immunogenicity data up to Month 7 and/or Month 12 may be also summarized as part of these interim analyses.
2. Separate 7-month interim analyses of safety, reactogenicity, and immunogenicity data collected from Visit Day 1 through Month 7 may be performed for phase A, phase B, and Arms 1 and 2 of phase C. The analyses are conducted on cleaned data and are reported on a treatment group level. Available safety or immunogenicity data up to Month 12 are also summarized as part of these interim analyses. These are partially unblinded analyses, as access to individual treatment assignments are restricted to pre-identified Sponsor study team members. The Investigators and site personnel remained blinded.
3. The final unblinded analysis of safety, reactogenicity, and immunogenicity data collected from Visit Day 1 through the end of study are conducted when the database is cleaned and locked.

Additional analyses of immunogenicity data may be performed to explore vaccine response.

Study safety oversight: Two safety monitoring boards including an IST and an independent SMC are organized to oversee the safety of the study.

### Results

Top line results of the 3-month interim analysis of the following data are detailed below: Phase A (n=27)
Safety of hCMV mRNA vaccine B and safety and immunogenicity of hCMV mRNA vaccine A for all subjects through end of study (12 months after the 3^{rd} vaccination) at all dose levels
   Dose-Escalation Phase B (n=15)
Safety for all subjects through Month 7 (1 month after the 3^{rd} vaccination) at the 30, 90 and 180 μg dose levels
Immunogenicity for all subjects through Month 3 (1 month after the 2^{nd} vaccination) at the 30, 90 and 180 μg dose levels
   Dose-Selection Phase B (n=104)
Safety and immunogenicity for all subjects through Month 3 (1 month after the 2^{nd} vaccination)

The purpose of this Phase I, first-in-human, randomized, observer-blind, placebo-controlled, dose-ranging study is to evaluate the safety and immunogenicity of hCMV mRNA vaccine A and the safety of hCMV mRNA vaccine B in healthy adults 18-49 years of age.

All subjects in Phase A and in dose-escalation Phase B were CMV-seronegative at enrollment, and approximately equal numbers of subjects in dose-selection Phase B were CMV-seronegative or CMV-seropositive at enrollment. Data are summarized for each phase separately unless otherwise specified. For Phase A, demographic and safety data are presented separately by receipt of hCMV mRNA vaccine B or hCMV mRNA vaccine A. For Phase B, demographic data are presented separately by dose-escalation and dose-selection subjects. Phase B safety and immunogenicity data are presented by dose-escalation and dose-selection subjects combined, and are summarized by CMV serostatus and overall.

### Summary of Results

Demographics

Demographics and baseline characteristics were generally balanced across treatment groups and Phase A and Phase B. The female:male ratio was consistent between Phase A and Phase B and was approximately 3:2.

Safety

Solicited safety data were collected through 7 days after each vaccination and are based on the Solicited Safety Set. Unsolicited events were collected through 28 days after each vaccination and are based on the Exposed Set.

### Overall

In Phase A, the hCMV mRNA vaccine B and hCMV mRNA vaccine A vaccines were generally well-tolerated though subject numbers were low, and the hCMV mRNA vaccine A vaccine in Phase B was generally well-tolerated at the two lower dose levels.

### Solicited Local Adverse Events

### Phase A

In Phase A, injection site pain was the most common solicited local AE reported in up to 100% of hCMV mRNA vaccine B and hCMV mRNA vaccine A recipients after each of the 3 vaccinations in a generally dose-related manner, and all were Grade 1-2.

### Phase B (Dose Escalation and Dose Selection Consolidated Data)

Injection site pain was also the most common solicited local AE in Phase B, reported in 76.7-100% of subjects after the 1^{st} vaccination and in 74.1-84.6% of subjects after the 2^{nd} vaccination and in a generally dose-related manner. Four of the 5 subjects reporting Grade 3 injection site pain after the Pt vaccination were CMV-seropositive, and the 8 reports of Grade 3 injection site pain after the 2^{nd} vaccination were equally distributed between CMV-seronegative and CMV-seropositive participants. Injection site erythema was reported in up to 15.4% of participants after the 1^{st} vaccination and up to 21.4% of participants after the 2^{nd} accination, with the higher rates occurring in the 180 μg treatment groups in both CMV-seronegative and CMV-seropositive participants. The single report of Grade 3 injection site erythema occurred in a CMV-seropositive participant in the 180 μg treatment group after the 2^{nd} vaccination. Rates of injection site swelling were low, reported by 3 subjects after the 1^{st} vaccination and 1 subject after and 2^{nd} vaccination, and all participants were CMV-seropositive.

Solicited local AE data after the 3^{rd} vaccination in Phase B are limited to the dose-escalation cohort. Injection site pain was the only AE reported, occurring only in CMV-seronegative subjects at all dose levels, and all were Grade 1-2.

Solicited Systemic Adverse Events

### Phase A

Headache, fatigue, myalgia, and arthralgia were the most common solicited systemic AEs. In hCMV mRNA vaccine B recipients, rates of solicited systemic AEs in subjects receiving hCMV mRNA vaccine B generally did not appear to be dose-related. Rates of solicited systemic AEs in subjects receiving hCMV mRNA vaccine A were generally higher at the 90 μg and 180 μg dose levels after the 1^{st} and 2^{nd} vaccinations. After the 1^{st} accination, overall rates of solicited systemic AEs were similar between hCMV mRNA vaccine B and hCMV mRNA vaccine A recipients. After the 2^{nd} vaccination, rates of headache, fatigue, myalgia, and arthralgia were higher in hCMV mRNA vaccine B recipients (50-75%) compared to hCMV mRNA vaccine A (16.7-33.3%). After the 3^{rd} vaccination, rates of these AEs were similar between hCMV mRNA vaccine B recipients (36.4-54.5%) and hCMV mRNA vaccine A recipients (36.4-54.5%). Fever was reported in only one hCMV mRNA vaccine A recipient across vaccinations. In hCMV mRNA vaccine B recipients, fever occurred only after the 2^{nd} and 3^{rd} vaccinations in 50% and 27.3% of subjects, respectively, and did not appear to be dose-related. All 17 of the Grade 3 solicited AEs in Phase A were systemic AEs, and all were reported in subjects receiving hCMV mRNA vaccine B after the 2^{nd} or 3^{rd} vaccinations.

It is of note that chills were not reported as solicited systemic AEs in Phase A, but were reported as solicited AEs in Phase B.

### Phase B (Dose Escalation and Dose Selection)

Headache, fatigue, myalgia, and chills were the most common solicited systemic AEs, were more commonly reported after the 2^{nd} vaccination (51.3-64.7%) compared to the 1^{st} vaccination (30.3-48.7%), occurred in a dose-related pattern, and at somewhat higher rates in CMV-seropositive subjects compared to CMV-seronegative subjects.

Rates of solicited systemic AEs were generally higher after the 2^{nd} vaccination compared to the 1^{st} vaccination in CMV-seronegative participants. Rates of fever were dose-related and higher after the 2^{nd} vaccination reported in 2% of CMV-seronegative participants and 33.3% of CMV-seropositive participants after the 1^{st} vaccination, and in 23.9% of CMV-seronegative participants and 41.2% of CMV-seropositive participants after the 2^{nd} vaccination. All 3 reports of Grade 3 fever after the 1^{st} vaccination and 5 of the 6 reports of Grade 3 fever after the 2^{nd} vaccination were reported in CMV-seropositive participants. Of the 56 Grade 3 solicited systemic AEs in Phase B, 19 were after the 1^{st} vaccination and 36 were after the 2^{nd} accination. Only one of the 19 Grade 3 events after the 1^{st} vaccination occurred in CMV-seronegative participants, but 35 of the 56 events after the 2^{nd} vaccination occurred in CMV-seronegative participants. The rates of Grade 3 solicited systemic AEs after the 1^{st} compared to the 2^{nd} vaccinations as related to CMV serostatus may reflect the 2^{nd} vaccination “boost” after the 1^{st} vaccination “prime” in CMV-seronegative participants compared to the immediate “boost” of the 1^{st} vaccination in CMV-seropositive participants.

Solicited systemic AE data after the 3^{rd} vaccination in Phase B are limited to the dose-escalation cohort. As after the 1^{st} and 2^{nd} accinations, headache, fatigue, myalgia, and chills were also the most frequently reported solicited systemic AEs after the 3^{rd} vaccination. All AEs were Grade 1-2, occurred in a generally dose-related manner, and were reported exclusively in the CMV-seronegative participants

Two subjects reported rash, one CMV-seronegative and one CMV-seropositive, and both were in the 180 μg treatment group in dose-selection Phase B. One of the subjects reported rash erroneously on the Diary Card, and the other subject experienced rash attributed to soy allergy and was deemed not related to study vaccine.

As of the data cutoff for this interim analysis, 26 of approximately 181 enrolled subjects had withdrawn from further vaccination. Of the 9 subjects who withdrew due to AEs experienced after study vaccination(s), all but one were enrolled in Phase B, most withdrew after their 2^{nd} vaccination, and approximately ⅔ were CMV-seropositive. Thirteen subjects withdrew from further vaccination for reasons other than AEs, and four subjects were lost to follow-up.

### Unsolicited Adverse Events

### Unsolicited AEs Through 28 Days After Each Vaccination

Of the 24 subjects randomized to vaccine treatment groups in Phase A, 18 reported unsolicited AEs. In the hCMV mRNA vaccine B group, 9 subjects reported 25 unsolicited AEs; of these, 8 subjects reported 23 unsolicited AEs deemed related to study product. In the hCMV mRNA vaccine A group, 9 subjects reported 12 unsolicited AEs; of these, 6 subjects reported 5 unsolicited AEs deemed related to study product. Two hCMV mRNA vaccine B recipients and 1 hCMV mRNA vaccine A recipient reported Grade 3 unsolicited AEs, all of which were deemed related to study product. No subjects experienced AEs that led to study discontinuation. The most common unsolicited AE was chills, reported in 12 participants (7 randomized to hCMV mRNA vaccine B and 5 randomized to hCMV mRNA vaccine A), with ≥1 participant in each of the treatment arms, and all were deemed related to study product A total of 5 subjects reported medically-attended AEs (3 randomized to hCMV mRNA vaccine B and 2 randomized to hCMV mRNA vaccine A), and the 2 medically-attended AEs deemed related to study product occurred in participants randomized to hCMV mRNA vaccine B. One participant randomized to placebo reported an unsolicited AE that was a medically-attended event that was not related to study product.

Of the 89 Phase B participants randomized to vaccine treatment groups, 38 participants (42.7%) reported 99 unsolicited adverse events; of these, 22 participants (24.7%) reported 68 events deemed related to study product. Of the 12 (13.5%) participants reporting Grade 3 unsolicited AEs, 7 (7.9%) reported events deemed related to study product. There was no pattern or difference in the distribution of AE categories between CMV-seronegative and CMV-seropositive subjects.

No subjects experienced AEs that led to study discontinuation.

The most frequent unsolicited AEs (fatigue, arthralgia, chills, myalgia, pyrexia) fell into Preferred Term categories collected as solicited AEs, but were categorized as unsolicited AEs due to being initially reported outside of the Diary Card collection tool. The next most frequent unsolicited AE related to study product was lymphadenopathy. Of the 7 placebo recipients reporting unsolicited AEs, 2 were CMV-seronegative and 5 were CMV-seropositive; 1 placebo recipient in the CMV-seropositive group reported an AE that was deemed related to study product. Six of the 8 subjects who reported medically-attended AEs were CMV-seropositive, with 1 CMV-seropositive subject in the 180 μg treatment group reporting an event that was deemed related to study product. Two subjects randomized to placebo reported medically-attended events, both were in the CMV-seropositive group and the events were not related to study product.

Overall, 9 subjects reported lymph node symptoms, 4 in Phase A and 5 in Phase B, and all were deemed related to study product. The 4 subjects in Phase A were randomized to hCMV mRNA vaccine B at either the 40 μg or 80 ᵢig treatment groups. In Phase B, 2 CMV-seronegative participants (1 each in the 90 μg and 180 μg treatment groups) and 3 CMV-seropositive participants in the 180 μg treatment group reported lymphadenopathy.

There have been no SAEs or AEs of special interest (AESI) in the study.

### Immunogenicity

Immunogenicity data are based on the Per Protocol (PP) immunogenicity set, and are reported as neutralizing antibody (nAb) against fibroblast infection and nAb against epithelial cell infection.

### Overall

Serum nAb responses after the 1^{st} and 2^{nd} accinations were dose-related and of comparable magnitude between Phase A subjects receiving hCMV mRNA vaccine A and Phase B CMV-seronegative subjects receiving hCMV mRNA vaccine A. A single vaccination boosted nAb in in CMV-seropositive subjects.

### Baseline Neutralizing Antibody

The baseline nAb GMTs against fibroblast infection and against epithelial cell infection in all treatment groups of Phase A and all treatment groups of CMV-seronegative subjects in Phase B were below the LLOQ (reported as 8, 0.5×LLOQ), indicating the absence of natural CMV infection prior to immunization.

In the Phase B CMV-seropositive group, the Baseline GMT of nAb against fibroblast infection was 1295.07 (95% CI=1022.39, 1640.48) and the Baseline GMT of nAb against epithelial cell infection was 5588.47 (95% CI=4252.06, 7344.91). These values represent the “natural CMV infection” benchmark against which immune responses in the CMV-seronegative group were compared. The CMV-positive benchmark nAb GMTs are comparable to healthy CMV-seropositive populations in a published report [Wang et al, Vaccine 2011:29].

hCMV mRNA vaccine A Neutralizing Antibody Responses

### Phase A

In Phase A participants, nAb GMT against fibroblast infection remained at baseline after the 1st vaccination, increased to ≥4 fold over baseline in all subjects after the 2nd vaccination in a dose-related manner, and remained at ≥4 fold over baseline after the 3rd vaccination with similar GMTs across dose levels. Decline in neutralizing antibodies titers was slower post 3^{rd} dose. Neutralizing antibodies against epithelial cell infection increased to ≥4 fold over baseline after all three vaccinations in all hCMV mRNA vaccine A treatment groups. At Month 12 (6 months after the 3^{rd} vaccination), nAb GMTs against fibroblast infection approached that of natural CMV infection at the 180 μg dose level (251.0, 418.6, and 1047.3 in the 30, 90, and 180 pg treatment groups, respectively), with GMT ranges overlapping the natural CMV infection benchmark at all dose levels. The nAb GMTs against epithelial cell infection at Month 12 exceeded the natural CMV infection benchmark at the 90 μg and 180 μg dose levels (5078.8, 13089.0, and 18915.9 in the 30, 90, and 180 μg treatment groups, respectively). Seroresponses (percentage of subjects with GMTs ≥4× baseline titer) were 100% across treatment groups for both nAb against fibroblast and against epithelial cell infection at Month 3 (after the 2^{nd} vaccination and Month 7 (after the 3^{rd} vaccination) and remained at 100% at Month 12 (6 months after the 3^{rd} vaccination).

### Phase B CMV-Seronegative Participants

In Phase B CMV-seronegative participants, GMTs against fibroblast and against epithelial cell infection after the P^{t} and 2^{nd} vaccinations were generally similar to or exceeded that of Phase A. Phase B nAb GMT against fibroblast infection increased in a dose-related manner after the 1^{st} vaccination, and increased further in a dose-related manner after the 2^{nd} vaccination to levels similar to the natural CMV infection benchmark in the 90 μg and 180 μg treatment groups (1140.6 and 1263.6, respectively). The nAb GMT against epithelial cell infection also increased in a dose-related manner after the 1^{st} vaccination, and increased further in a dose-related manner after the 2^{nd} vaccination to levels exceeding the natural CMV infection benchmark in the 90 μg and 180 μg treatment groups (15,305.3 and 30,742.9, respectively).

After the 2^{nd} vaccination, the increases in nAb GMTs compared to baseline were robust across all dose levels, with GMRs of 38.15, 142.57, and 157.96 for nAb against fibroblast infection, and 407.86, 1,913.17, and 3,842.87 for nAb against epithelial cell infection in the 30, 90, and 180 μg treatment groups, respectively. Seroresponses (percentage of subjects with GMTs ≥4x baseline titer) at Month 3 were also robust, with 92.9%, 100%, and 100% of subjects having GMTs ≥4× baseline for nAb against fibroblast infection in the 30, 90, and 180 μg treatment groups, respectively, and 100% of subjects having nAb against epithelial cell infection in all treatment groups.

### Phase B CMV-Seropositive Participants

In Phase B CMV-seropositive subjects, the 1^{st} vaccination boosted nAb against fibroblast infection and against epithelial cell infection in a dose-related manner, with nAb GMRs against fibroblast infection of 2.43, 2.66, and 2.83, and against epithelial cell infection of 6.85, 6.93, and 9.26 in the 30, 90, and 180 μg treatment groups, respectively. The 2^{nd} vaccination slightly increased nAb GMRs against fibroblast infection at the two higher dose levels and substantially increased with GMRs against epithelial cell infection at all dose levels, with nAb GMRs against fibroblast infection of 2.30, 3.00, and 4.08, and nAb GMRs against epithelial cell infection of 13.15, 9.91, and 19.36 in the 30, 90, and 180 μg treatment groups, respectively. These results suggest that the 2^{nd} vaccination slightly impacted the nAb response against fibroblast infection, and substantially impacted the nAb response against epithelial cell infection.

### Conclusions

These interim data support the following conclusions:

The overall safety profile of hCMV mRNA vaccine A vaccine is similar to that of licensed vaccines, however rates of Grade 3 solicited AEs were higher in the 180 μg treatment groups. In CMV-seronegative subjects, solicited AE rates were higher after the 2^{nd} vaccination compared to the P^{t} vaccination possibly due to a “boosting” effect of the 2^{nd} vaccination. Solicited AE rates after the P^{t} vaccination in CMV-seropositive subjects were higher than in CMV-seronegative subjects, suggesting a “boosting” effect after a single vaccination in naturally-infected individuals. An unsolicited AE was lymphadenopathy/lymph node pain, reported only in subjects randomized to vaccine treatment groups, was transient in nature, usually occurred within a few days after vaccination, and possibly related to immune activation after vaccination.

Serum nAb GMTs increased after each vaccination in a dose-related manner, and were numerically similar between Phase A subjects receiving hCMV mRNA vaccine A and Phase B CMV-seronegative subjects receiving hCMV mRNA vaccine A. After the 2^{nd} vaccination in Phase B, nAb GMT against fibroblast infection approached the benchmark of natural CMV infection in the 90 μg and 180 μg treatment groups, and nAb GMT against epithelial cell infection exceeded the benchmark of natural CMV infection in all treatment groups. Neutralizing antibody GMTs were sufficiently boosted in CMV-seropositive subjects after a single vaccination, which increased further after the second vaccination for nAb GMTs against epithelial cell infection. In Phase A and Phase B CMV-seronegative participants, seroresponses (percentage of subjects with GMTs ≥4x baseline titer) were robust through the 2^{nd} vaccination, and continued to be robust through 12 months in Phase A, suggesting sustained antibody responses to hCMV mRNA vaccine A through at least 6 months after the 3^{rd} vaccination.

7-Month Interim Analysis of Safety and Immunogenicity of hCMV mRNA Vaccine A

In this interim analysis, the following data were reviewed and analyzed: (i) safety and immunogenicity through Month 7 (1 month after the 3^{rd} vaccination) for the 30, 90, and 180 μg dose groups and placebo in dose-escalation Phase B (n=15); (ii) safety and immunogenicity through Month 7 (1 month after the 3^{rd} vaccination) for the 30, 90, and 180 μg dose groups and placebo in dose-selection Phase B (n=104); and (iii) safety and immunogenicity through Month 3 (1 month after the 2^{nd} vaccination) for the 300 μg dose group and placebo in sentinel-Expansion Phase C (n=35).

Immunogenicity was reported as neutralizing antibody (nAb) against epithelial cell and against fibroblast infection for all subjects in Phase B and Phase C. Cell-mediated immunogenicity as measured by IFN-γ-secreting gB-specific T-cells by ELISpot was reported in 13 dose-escalation Phase B subjects.

### Summary of Results

### 1. Demographics

Demographics and baseline characteristics were generally balanced across the treatment groups of Phase B and Phase C with the exception of higher age in the Phase C placebo group (42.5±6.2 years in the placebo group and 33.3±8.7 years in the 300 μg treatment group). The proportion of females enrolled in Phase B and Phase C was generally consistent across the treatment groups. At least 80% of all treatment groups across Phase B and Phase C were white.

### 2. Safety

Solicited safety data were collected through 7 days after each vaccination and are based on the Solicited Safety Set. Unsolicited events were collected through 28 days after each vaccination and are based on the Exposed Set.

Overall, hCMV mRNA vaccine A was generally well tolerated. The proportion of subjects reporting solicited adverse reactions (ARs) in the Phase C (300 μg) hCMV mRNA vaccine A treatment group was comparable to that of the Phase B 180 μg hCMV mRNA vaccine A treatment group for both CMV-seronegative and CMV-seropositive groups.

1. Solicited Local Adverse Reactions (AR):

The most commonly reported solicited local AR after the 1^{st} or 2^{nd} accinations was injection site pain, which was generally reported in CMV-seronegative subjects as frequently as in CMV-seropositive subjects and reported by higher proportions of subjects in either the 180 μg or 300 μg hCMV mRNA vaccine A treatment groups. The subjects were generally distributed across hCMV mRNA vaccine A treatment groups. In Phase B subjects after the 3^{rd} vaccination, the rate and severity of injection site pain reported was generally decreased compared to the 2^{nd} vaccination. Injection site pain was reported in 0-14% of placebo recipients across treatment groups, and none were of Grade 3 severity.

The proportions of subjects reporting injection site erythema after the Pt or 2^{nd} vaccinations were generally low, with rates ranging 0-22% in CMV-seronegative subjects and 0-18% in CMV-seropositive subjects across treatment groups. All 7 subjects reporting injection site erythema after either the 1^{st} or 2^{nd} _{vacc}iₙₐtions were in the 180 μg or 300 μg hCMV mRNA vaccine A treatment groups. One CMV-seronegative subject in the 300 μg treatment group and one CMV-seropositive subject in the 180 μg treatment group reported Grade 3 injection site erythema after the 2^{nd} vaccination. In Phase B subjects after the 3^{rd} ᵥaccination, the rate and severity of injection site erythema reported did not substantially increase after the 3^{rd} vaccination compared to the 2nd vaccination. No subjects in the placebo group reported injection site erythema.

The proportions of subjects reporting injection site swelling after the 1^{st} or 2^{nd} vaccinations were also low, with rates ranging 0-25% in CMV-seronegative subjects and 0-14% in CMV-seropositive subjects across treatment groups. More subjects reporting injection site swelling after either the 1^{st} or 2^{nd} _{vacc}iₙₐtions were in the 180 μg or 300 μg hCMV mRNA vaccine A treatment groups. In Phase B subjects after the 3^{rd} vaccination, the rates of injection site swelling remained low after the 3^{rd} vaccination. One CMV-seronegative subject in the 300 μg treatment group reported Grade 3 injection site swelling after the 2^{nd} vaccination. No subjects in the placebo group reported injection site swelling.

2. Solicited Systemic Adverse Reactions:

Headache, fatigue, myalgia, and chills were the most common solicited systemic ARs across all vaccinations and all hCMV mRNA vaccine A CMV-seronegative and CMV-seropositive hCMV mRNA vaccine A treatment groups. There were more Grade 3 ARs reported in CMV-seropositive subjects. The majority of Grade 3 ARs occurred after the 2nd vaccination in CMV-seronegative subjects but was more balanced between the 1st and 2nd vaccinations in CMV-seropositive subjects, which may reflect an immunologic “boost” of a 2nd vaccination after the “prime” of a 1^{st} vaccination in CMV-seronegative hCMV mRNA vaccine A recipients, compared to the immediate “boost” after the 1^{st} vaccination in CMV-seropositive hCMV mRNA vaccine A recipients. See (Table 1 and Table 2).

As of the data cutoff for this interim analysis, 10 of 181 enrolled subjects in this study had withdrawn from further vaccination due to ARs, and all of these subjects continued to be followed in the study for safety. Of the 10 subjects, 3 were in the 30 μg treatment group (2 of these were CMV-seropositive), no subjects were in the 90 μg treatment group, 5 were in the 180 μg treatment group (4 of these were CMV-seropositive), 1 was a CMV-seropositive subject in the Dose-selection Phase B placebo group, 1 was a CMV-seronegative subject in the hCMV mRNA vaccine B treatment group of Phase A. Seven of the 10 subjects reported >1 Grade 3 solicited AR after the previous vaccination, and the remaining 3 reported ARs that were Grade 2 or lower after the previous vaccination. All but one of the 10 subjects were enrolled in Phase B, most subjects withdrew after the 2^{nd} vaccination, and the majority were CMV-seropositive.

Of the subjects who have discontinued from the study, 11 were in Phase B mRNA treatment groups, 6 were in Phase C mRNA treatment groups, and 5 were Phase B placebo recipients. Subject discontinuations were distributed across treatment groups, and the majority of the reasons for discontinuation were “lost to follow-up” or “withdrawal by subject”.

3. Unsolicited Adverse Events:

### Unsolicited AEs Through 28 Days After Each Vaccination

One CMV-seronegative placebo recipient in Phase C experienced 7 unrelated SAEs due to a motor vehicle accident. There have been no AEs of special interest (AESI) in the study.

Lymph node symptoms were reported in 7 subjects: 6 (7%) Phase B subjects and 1 (3.4%) Phase C subject. All 7 subjects received hCMV mRNA vaccine A. Five of the 7 subjects were in either the 180 μg or 300 μg treatment groups. Four of the 7 subjects were CMV-seronegative and reported symptoms after the 2^{nd} or 3^{rd} ᵥaccination, and the 3 of the 7 subjects were CMV-seropositive and reported symptoms after the 1^{st} or 2^{nd} vaccination. All events were assessed as related to vaccination. At safety reviews, this AE was generally described as transient axillary swelling ±tenderness of mild to moderate severity that always occurred on the same side as the vaccinated arm.

Laboratory Abnormalities:

In Phase B, the following Grade 3 shifts from baseline of safety laboratory parameters were reported: hemoglobin in 2 placebo and 2 hCMV mRNA vaccine A recipients, hypoglycemia in 2 hCMV mRNA vaccine A recipients, high leukocytes in 1 placebo recipient, and elevated PTT in 1 hCMV mRNA vaccine A recipient. In Phase C, the following Grade 3 shifts from baseline of safety laboratory parameters were reported: hemoglobin in 1 placebo and hCMV mRNA vaccine A recipients, and hyperglycemia in 2 hCMV mRNA vaccine A recipients.

There were 2 subjects reporting Grade 4 shifts from baseline of safety laboratory parameters: hypoglycemia in a Phase C placebo recipient with Grade 3 hypoglycemia at baseline and, as reported in the Interim Analysis, one subject reported asymptomatic Grade 4 PTT elevation at 7 days post-2^{nd} vaccination which was deemed related and triggered study pause. This subject was noted to have persistent Grade 1 elevations in PTT values prior to the Grade 4 PTT. On re-testing, this subject's PTT result was within normal limits. The Safety Monitoring Committee reviewed the event and recommended to continue the study without modifications.

### 3. Immunogenicity

Neutralizing antibody data against epithelial cell infection and against fibroblast infection were based on the Per Protocol (PP) Immunogenicity Set, and were reported as geometric mean titer (GMT) and geometric mean ratio (GMR, defined as the ratio of baseline/post-baseline titers). The microneutralization assay for measurement of nAb titers against epithelial cell infection utilized CMV isolate VR1814 and ARPE-19 cells, and for measurement of nAb titers against fibroblast infection CMV isolate AD169 and HEL299 cells were utilized.

Cell mediated immunogenicity data were based on the Cell-mediated Immunogenicity Set and were reported as SFC/10⁶ PBMC.

1. Overall

In CMV-seronegative subjects, serum nAb titers increased with hCMV mRNA vaccine A dose after each of the first 2 vaccinations and continued to increase after the 3^{rd} vaccination in Phase B. In CMV-seropositive subjects, a single vaccination boosted nAb titers in a dose-related manner across all hCMV mRNA vaccine A treatment groups, which further boosted after the 2^{nd} and 3^{rd} vaccinations in Phase B and after the 2^{nd} accination in Phase C. In the Phase B dose-escalation subjects, post-vaccination gB-specific T-cell activation was observed at all dose levels.

Neutralizing antibodies against epithelial cell infection and against fibroblast infection increased in a dose-related manner and increased with subsequent hCMV mRNA vaccine A vaccinations within each hCMV mRNA vaccine A treatment group through Month 7 (1 month after the 3rd vaccination) in both CMV-seronegative and CMV-seropositive participants.

In CMV-seronegative participants at Month 12 (6 months after the 3rd vaccination), nAb GMTs against epithelial infection remained at least 3.5-fold higher than the natural infection benchmark, and nAb GMTs against fibroblast infection approximated that of the natural infection benchmark in the in the 90pg and 180pg treatment groups. In CMV-seropositive participants at Month 12, nAb GMRs against epithelial infection were 14-fold to 31-fold over baseline and against fibroblast infection were 6-fold to 8-fold over baseline.

2. Natural CMV Infection Neutralizing Antibody Titer Benchmarks:

The baseline nAb GMT of all per-protocol CMV-seropositive subjects in Phase B and C was used as the “natural infection” benchmark values against which immune responses in the CMV-seronegative group were compared. The benchmark nAb GMT against epithelial cell infection was 5,917 (95%CI 4644,7540) and the benchmark nAb GMT against fibroblast infection was 1,449 (95%CI 1167,1800). These values are comparable to healthy CMV-seropositive populations. (Wang et al, Vaccine 2011:29.)

The baseline nAb GMTs against fibroblast infection and against epithelial cell infection in all CMV-seronegative treatment groups were below the LLOQ (reported as 8, 0.5 x LLOQ), indicating the absence of natural CMV infection prior to immunization.

3. CMV-Seronegative Subjects:

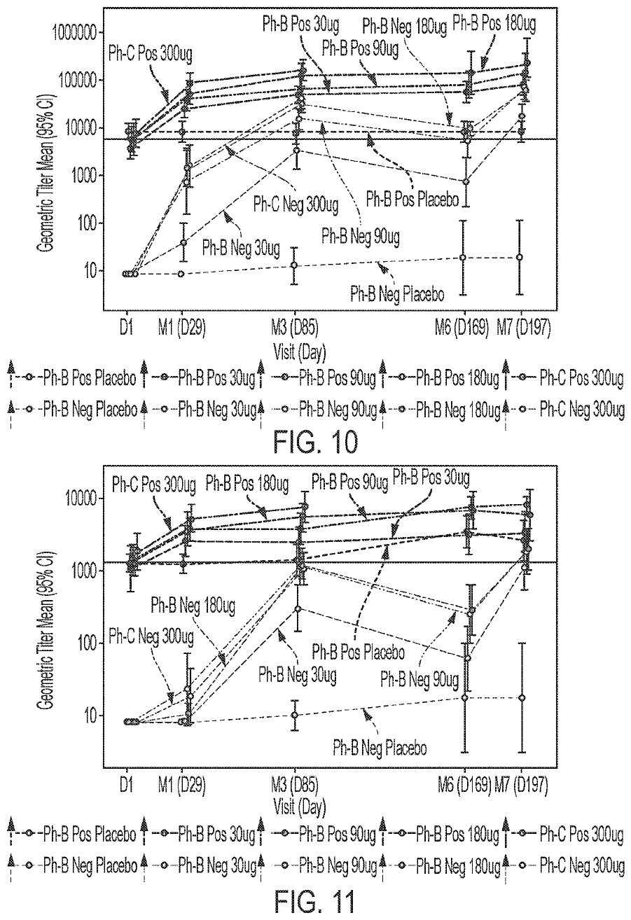

Baseline nAb GMTs against fibroblast infection and against epithelial cell infection in all treatment groups of Phase B and Phase C were below lower limits of quantitation (LLOQ) and were reported as 8 (0.5×LLOQ), indicating the absence of natural CMV infection prior to enrollment. Neutralizing antibody responses were reported after each of the 3 vaccinations for Phase B and after the first 2 vaccinations for Phase C (Table 3, FIG. 10, FIG. 11).

Neutralizing antibody GMT against epithelial cell infection increased in a dose-related manner and after each subsequent vaccination within hCMV mRNA vaccine A treatment groups. One month after the 2^{nd} vaccination, nAb GMT against epithelial cell infection were 3,263; 15,305; 30,743; and 43,564 in the 30 μg, 90 μg, 180 μg, and 300 μg treatment groups, respectively, which exceeded the natural infection benchmark in the 90 μg, 180 μg, and 300 μg treatment groups. One month after the 3^{rd} ᵥaccination, nAb GMTs against epithelial cell infection increased further to 16,587; 63,929; and 62,118 in the 30 μg, 90 μg, 180 μg treatment groups, respectively, which exceeded the natural infection benchmark in all hCMV mRNA vaccine A treatment groups.

Neutralizing antibody against fibroblast infection generally increased in a dose-related manner and after each subsequent vaccination within hCMV mRNA vaccine A treatment groups. One month after the 3^{rd} vaccination, nAb GMTs against fibroblast infection were 1,131; 1,890; and 2,029 in the 30 μg, 90 μg, 180 μg treatment groups, respectively, exceeding the natural infection benchmark in the 90 μg, 180 μg treatment groups.

At 1 month after the 2^{nd} vaccination, nAb seroresponse (percentage of subjects with nAb titer ≥4x baseline titer) against epithelial cell infection was achieved in 100% of subjects in all treatment groups across Phase B and Phase C, and nAb seroresponse against fibroblast infection was achieved in 93% of subjects in the 30 μg treatment group and in 100% of subjects in the 90 μg, 180 μg, and 300 μg treatment groups.

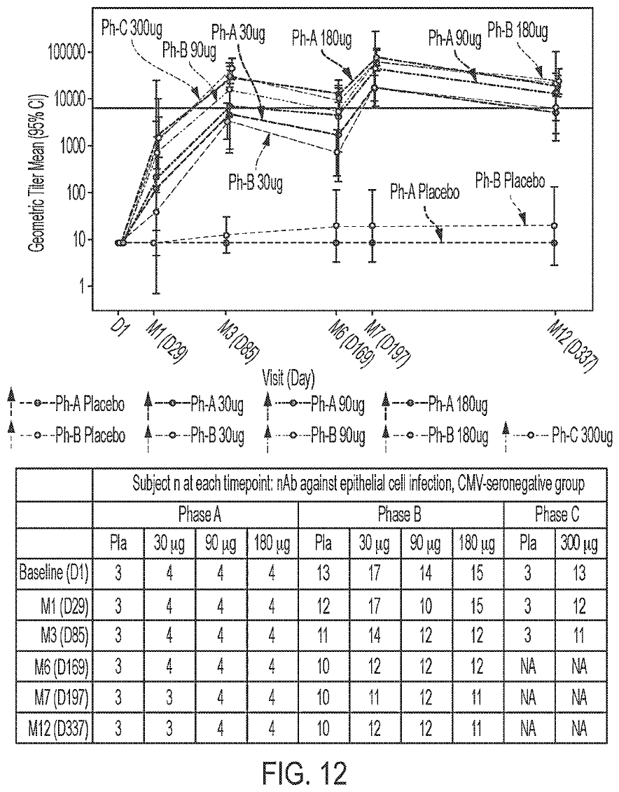

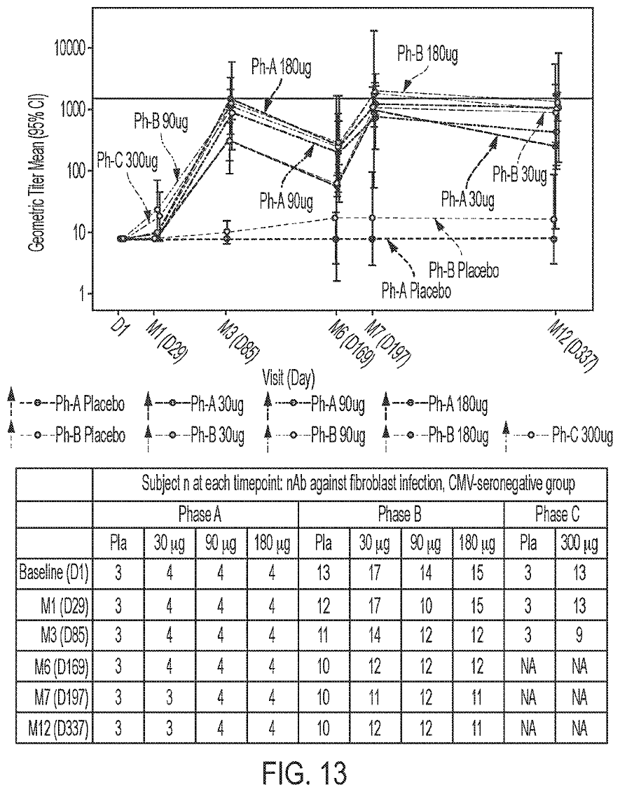

Table 3, FIG. 12 and FIG. 13 summarize nAb data through Month 12 in the Phase B treatment groups. At Month 7 (one month after the 3^{rd} ᵥaccination) GMTs of nAbs against epithelial cell infection in the 30 μg, 90 μg and 180 μg treatment groups were 16,587 (95% CI 9,186; 29,952), 63,929 (95% CI 38,441; 106,317), and 62,118 (95% CI 33,829; 114,065), which represented GMTs that were 2.8-fold, 10.8-fold, and 10.5-fold higher than the natural infection benchmark At Month 12 (6 months after the 3rd vaccination), GMTs in the 30 μg, 90 μg and 180 μg treatment groups were 6,414 (95% CI 3,457; 10,908), 21,211 (95% CI 13,689; 32,865), and 23,020 (95%CI 12,473; 42,484), respectively, indicating GMTs were maintained at 3.6-fold and 3.9-fold higher than the natural infection benchmark 90 μg and 180g treatment groups at 6 months after the last vaccination (Table 3).

At Month 7 (one month after the 3rd vaccination), GMTs of nAb against fibroblast infection in the 30 μg, 90 μg and 180 μg treatment groups were 1,131 (95%CI 531; 2,408), 1,890 (95%CI 918; 3,890), and 2,029 (95%CI 1,042; 3,953), exceeding the natural infection benchmark by 1.3-fold to 1.4-fold in the 90 μg and 180 μg treatment groups. At Month 12 (6 months after the 3rd vaccination) GMTs of nAb against fibroblast infection in the 30 μg, 90 μg and 180m treatment groups were 911 (95%CI 457; 1,816), 970 (95%CI 516; 1,823), and 1,308 (95%CI 680; 2,518), approximating the natural infection benchmark for the 90 μg and 180 μg treatment groups at 6 months after the last vaccination (Table 3).

At 12 months, individual titers of ≥4-fold over baseline were maintained in 100% of participants for both nAbs against epithelial cell infection as well as nAbs against fibroblast infection in the 30 μg, 90 μg and 180 μg hCMV mRNA vaccine A treatment groups.

TABLE 3
Neutralizing Antibody Responses, CMV-seronegative Participants, Per-protocol Set
Neutralizing Antibodies Against Epithelial Ce11 Infection, CMV-seronegative Participants
natural infection benchmark GMT = 5,917

| | Phase B | | | | Phase C | |
|---|---|---|---|---|---|---|
| | Placebo* | 30 μg | 90 μg | 180 μg | Placebo | 300 μg |
| | N = 13 | N = 17 | N = 14 | N = 15 | N = 3 | N = 13 |
| GMT baseline | 8 | 8 | 8 | 8 | 8 | 8 |
| | n = 13 | n = 17 | n = 14 | n = 15 | n = 3 | n = 13 |
| GMT Month 1 | 8 | 37 | 708 | 1,387 | 8 | 1,481 |
| | n = 12 | n = 17 | n = 10 | n = 15 | n = 3 | n = 12 |
| GMT Month 3 | 12 | 3,263 | 15,305 | 30,743 | 8 | 43,564 |
| | n = 11 | n = 14 | n = 12 | n = 12 | n = 3 | n = 11 |
| GMT Month 7 | 18 | 16,587 | 63,929 | 62,118 | NA | NA |
| | n = 10 | n = 11 | n = 12 | n = 11 | | |
| GMT Month 12 | 19 | 6,141 | 21,211 | 23,020 | NA | NA |
| | n = 10 | n = 12 | n = 12 | n = l 1 | | |
| GMT/benchmark Month 3 | — | 0.6 | 2.6 | 5.2 | — | 7.4 |
| GMT/benchmark Month 7 | — | 2.8 | 10.8 | 10.5 | NA | NA |
| GMT/benchmark Month 12 | — | 1.0 | 3.6 | 3.9 | NA | NA |

Neutralizing Antibodies Against Fibroblast Infection, CMV-seronegative Participants
natural infection benchmark GMT = 1,449

| | Phase B | | | | Phase C | |
|---|---|---|---|---|---|---|
| | Placebo* | 30 μg | 90 μg | 180 μg | Placebo | 300 μg |
| | N = 13 | N = 17 | N = 14 | N = 15 | N = 3 | N = 13 |
| GMT baseline | 8 | 8 | 8 | 8 | 8 | 8 |
| | n = 13 | n = 17 | n = 14 | n = 15 | n = 3 | n = 13 |
| GMT Month 1 | 8 | 8 | 24 | 10 | 8 | 18 |
| | n = 12 | n = 17 | n = 10 | n = 15 | n = 3 | n = 12 |
| GMT Month 3 | 10 | 305 | 1,141 | 1,264 | 8 | 1,065 [symbol] |
| | n = 11 | n = 14 | n = 12 | n = 12 | n = 3 | n = 11 |
| GMT Month 7 | 17 | 1,131 | 1,890 | 2,029 | NA | NA |
| | n = 10 | n = 11 | n = 12 | n = 11 | | |
| GMT Month 12 | 17 | 911 | 970 | 1,308 | NA | NA |
| | n = 10 | n = 12 | n = 12 | n = 11 | | |
| GMT/benchmark Month 3 | — | 0.2 | 0.8 | 0.9 | — | 0.8 |
| GMT/benchmark Month 7 | — | 0.8 | 1.3 | 1.4 | NA | NA |
| GMT/benchmark Month 12 | — | 0.6 | 0.7 | 0.9 | NA | NA |

Natural infection benchmark GMTs derived from all CMV-seropositive participant titers (Phase B and Phase C) at baseline.
GMT = geometric mean titer;
GMR = geometric mean ratio;
N = number of participants in any Per Protocol set;
n = number of participants contributing data at the corresponding timepoint.
*One CMV-seronegative placebo recipient has a seroconversion pattern onset between Month 1 and Month 3.
†One participant had indeterminate result at previous interim analysis, sample re-run per protocol and included in this interim analysis.

4. CMV-seropositive Subjects:

Baseline nAb GMTs against epithelial cell infection ranged 3,614-7,179 in the hCMV mRNA vaccine A treatment groups and 6,900-8,169 in the placebo groups, indicating presence of natural CMV infection prior to enrollment. Neutralizing antibody responses were reported after each of the 3 vaccinations for Phase B and after the first 2 vaccinations for Phase C (Table 4, FIG. 10, FIG. 11).

The first vaccination boosted nAb in a dose-related manner across all hCMV mRNA vaccine A treatment groups, with nAb GMTs against epithelial cell infection of 24,752; 39,020; 52,775; and 84,628 and nAb GMTs against fibroblast infection of 2,654; 3,885; 3,879; and 5,419 in the 30 μg, 90 μg, 180 μg, and 300 μg treatment groups, respectively. Subsequent vaccinations further boosted nAb across hCMV mRNA vaccine A treatment groups with nAb GMTs against epithelial cell infection of 49,390; 62,400; 119,829, and 156,583 and nAb GMTs against fibroblast infection of 2,517; 3,891; 5,578; and 7,788 at 1 month after the 2^{nd} vaccination in the 30 μg, 90 μg, 180 μg, and 300 μg treatment groups, respectively. One month after the 3^{rd} vaccination, nAb GMTs against epithelial cell infection were 76,914; 141,020; and 211,503 and nAb GMTs against fibroblast infection were 3,412; 8,433; and 6,098 in the 30 μg, 90 μg, and 180 μg treatment groups, respectively.

Accordingly, GMRs also increased. At 1 month after the 2^{nd} vaccination, nAb GMRs against epithelial cell infection were 14.4, 9.9, 19.4, and 17.3 and nAb GMRs against fibroblast infection were 2.5, 3.0, 4.1, and 3.8 in the 30 μg, 90 μg, 180 μg, and 300 μg treatment groups, respectively. After the 3^{rd} vaccination, nAb GMRs against epithelial cell infection were 26.2, 22.4, and 40.8 and nAb GMR against fibroblast infection were 4.0, 6.5, and 3.9 in the 30 μg, 90 .t.g, 180 μg treatment groups, respectively.

Table 4 summarizes nAb data through Month 12 in CMV-seropositive participants in the Phase B treatment groups. At Month 7 (one month after the 3rd vaccination), GMTs of nAbs against epithelial cell infection in the 30 μg, 90 μg and 180 μg treatment groups were 76,914 (95%CI 49,001; 120,727), 141,020 (95%CI 57,649; 344,960), and 211,503 (95%CI 58,207; 768,525), yielding corresponding GMRs of 26.2, 22.4, and 40.8. At Month 12 (6 months after the 3rd vaccination) GMTs were 44,186 (95%CI 26,281; 74,287), 87,999 (95%CI 44,012; 175,948), and 162,749 (95%CI 69,529;380,953) in the 30m, 90.tg and 180 μg treatment groups, respectively, resulting in GMRs of 14.6, 13.98, and 31.4, respectively, at 6 months after the last vaccination (Table 4).

At Month 7 (one month after the 3rd vaccination), GMTs of nAbs against fibroblast infection were 3,412 (95%CI 1,924; 6,052); 8,433 (95%CI 6,582; 10,804); and 6,427 (95%CI 3,426; 12,057) in the 30m, 90 μg, and 180 μg treatment groups, respectively, resulting in GMRs ranging 4-6.5 at the Month 7 timepoint. At Month 12 (6 months after the 3rd vaccination), GMTs were 7,170 (95%CI 4,052; 12,686), 7,640 (95%CI 4,602; 12,685), and 10,030 (95%CI 7,577;13,276) in the 30 μg, 90 μg and 180 μg treatment groups, respectively, resulting in GMRs of 8.1, 5.9, and 6.3, respectively, after the 3rd vaccination (Table 4).

For all participants, the Month 12 nAb data were generated in assay runs that were separate from the assay runs generating the nAb data at all previous timepoints (Baseline through Month 7). In 3 of the 4 CMV-seropositive Phase B treatment groups (30 μg and 180 μg hCMV mRNA vaccine A and placebo), the GMT of nAb against fibroblast infection trended higher at Month 12 compared to Month 7, whereas the GMT of nAb against epithelial cells trended lower (Table 4).

TABLE 4
Neutralizing Antibody Responses, CMV-Seropositive Participants, Per-protocol Set

| Phase B | | | | Phase C | |
|---|---|---|---|---|---|
| Placebo | 30 μg | 90 μg | 180 μg | Placebo | 300 μg |
| N = 14 | N = 13 | N = 12 | N = 13 | N = 3 | N = 13 |

Neutralizing Antibodies Against Epithelial Cell Infection, CMV-seronegative Participants
natural infection benchmark GMT = 5,917

| | | | | | | |
|---|---|---|---|---|---|---|
| GMT baseline | 8,169 | 3,614 | 5,634 | 5,700 | 6,900 | 7,179 |
| | n = 14 | n = 13 | n = 12 | n = 13 | n = 3 | n = 13 |
| GMT Month1 | 7,891 | 24,752 | 39,020 | 52,775 | 6,673 | 184,628 |
| | n = 14 | n = 13 | n = 12 | n = 13 | n = 3 | n = 13 |
| GMT Month 3 | 7,490 | 49,390 | 62,400 | 119,829 | 5,974 | 156,583 |
| | n = 12 | n = 11 | n = 9 | n = 9 | n = 3 | n = 9 |
| GMT Month 7 | 7,647 | 76,914 | 141,020 | 211,503 | NA | NA |
| | n = 12 | n = 10 | n = 9 | n = 6 | | |
| GMT Month 12 | 7,803 | 44,186 | 87,999 | 162,749 | NA | NA |
| | n = 11 | n = 11 | n = 9 | n = 6 | | |
| GMT/benchmark Month 3 | 0.9 | 14.4 | 9.9 | 19.4 | 0.9 | 17.33 |
| | nₐ = 12 | nₐ = 11 | nₐ = 9 | nₐ = 9 | nₐ = 3 | nₐ = 9 |
| GMT/benchmark Month 7 | 0.98 | 26.2 | 22.4 | 40.8 | NA | NA |
| | nₐ = 12 | nₐ = 10 | nₐ = 9 | nₐ = 6 | | |
| GMT/benchmark Month 12 | 1.0 | 14.6 | 13.98 | 31.4 | NA | NA |
| | nₐ = 11 | nₐ = 11 | nₐ = 9 | nₐ = 6 | | |

Neutralizing Antibodies Against Fibroblast Infection, CMV-seronegative Participants
natural infection benchmark GMT = 1,449

| | | | | | | |
|---|---|---|---|---|---|---|
| GMT baseline | 1,298 | 1,094 | 1,458 | 1,371 | 3,631 | 1,836 |
| | n = 14 | n = 13 | n = 12 | n = 13 | n = 3 | n = 13 |
| GMT Month1 | 1,278 | 2,654 | 3,885 | 3,879 | 3,382 | 5,419 |
| | n = 14 | n = 13 | n = 12 | n = 13 | n = 3 | n = 13 |
| GMT Month 3 | 1,451 | 2,517 | 3,891 | 5,578 | 2,797 | 7,788 |
| | n = 12 | n = 11 | n = 9 | n = 9 | n = 3 | n = 9 |
| GMT Month 7 | 2,673 | 3,412 | 8,433 | 6,427* | NA | NA |
| | n = 12 | n = 10 | n = 9 | n = 6 | | |
| GMT Month 12 | 5,240 | 7,170 | 7,640 | 10,030 | NA | NA |
| | n = 11 | n = 11 | n = 9 | n = 6 | | |
| GMT/benchmark Month 3 | 1.1 | 2.5 | 3.0 | 4.1 | 0.8 | 3.8 |
| | nₐ = 12 | nₐ = 11 | nₐ = 9 | nₐ = 9 | nₐ = 3 | nₐ = 9 |
| GMT/benchmark Month 7 | 2.2 | 4.0 | 6.5 | 4.1* | NA | NA |
| | nₐ = 11 | nₐ = 10 | nₐ = 9 | nₐ = 6 | | |
| GMT/benchmark Month 12 | 4.4 | 8.1 | 5.9 | 6.3 | NA | NA |
| | nₐ = 11 | nₐ = 11 | nₐ = 9 | nₐ = 6 | | |

GMT = geometric mean titer; natural infection benchmark GMTs derived from all CMV-seropositive participant titers (Phase B and Phase C) at baseline,
GMR = geometric mean ratio;
N = number of participants in any Per Protocol set;
n = number of participants contributing data at the corresponding time point;
na = number of participants with non-missing data at baseline and the corresponding time point
*One participant had indeterminate result at previous interim analysis, sample re-run per protocol and included in this interim analysis.

5. Cell-Mediated Immunogenicity

The hCMV mRNA vaccine A elicited detectable gB-specific T-cell responses in the small subset of 13 Dose-escalation Phase B subjects (30 μg, 90 μg, and 180 μg treatment groups). One week after the 2^{nd} vaccination, mean responses across hCMV mRNA vaccine A treatment groups ranged 153-720 SFC/10⁶PBMC (mean response 146-687 SFC/10⁶PBMC over baseline). One week after the 3^{rd} vaccination, mean responses across treatment groups ranged 72-971 SFC/10⁶ PBMC (mean response 65-922 SFC/10⁶ PBMC over baseline).

### Conclusions

These interim data support the following conclusions:
Safety conclusions:
   a. The overall rates of solicited systemic ARs generally numerically increased with hCMV mRNA vaccine A dose through 180 μg and were generally similar between the 180 μg and 300 μg treatment groups.
   b. In general, higher proportions of CMV-seropositive subjects reported solicited ARs compared to CMV-seronegative subjects.
   c. In CMV-seronegative subjects, the frequencies of solicited ARs were generally numerically higher after the 2^{nd} vaccination compared to the 1st vaccination, a possible clinical indication of an immunologic “boosting” effect with the 2nd vaccination after the “prime” of the 1st vaccination.
   d. In CMV-seropositive subjects, the frequencies of solicited ARs were generally numerically higher after the 1^{st} vaccination, a possible clinical manifestation of an immunologic “boosting” effect in subjects with a history of natural CMV infection prior to enrollment.
   e. Seven subjects reported transient, mild-moderate axillary lymph node symptoms ipsilateral to the vaccinated arm, which may represent a benign clinical manifestation of vaccine-induced immune activation.
   f. In CMV-seronegative participants after the 3rd vaccination, the frequency of solicited local and systemic ARs was generally higher in the 300 μg treatment group compared to the 180 μg treatment group. Proportions of participants reporting severe solicited ARs were similar between the 180 μg and 300 μg treatment groups.
   g. In CMV-seropositive participants after the 3rd vaccination, there was no overall difference in the frequency of solicited local and systemic ARs in the 300 μg treatment group compared to the 180 μg treatment group. Proportions of participants reporting severe solicited ARs was generally numerically higher in the 300 μg treatment groups compared to the 180 μg treatment groups.

### Immunogenicity Conclusions:

a. Neutralizing antibodies against epithelial cell infection (a measure of immune response to intact CV Pentamer) and against fibroblast infection (a measure of immune response to CMV gB antigen) responded in a dose-related manner and increased with subsequent hCMV mRNA vaccine A vaccinations within each hCMV mRNA vaccine A treatment group.
b. Neutralizing antibody responses against epithelial cell infection were robust. In CMV-seronegative subjects, GMTs exceeded the natural infection benchmark in the 90 μg, 180 lig, and 30014 treatment groups at 1 month after the 2^{nd} vaccination and in all Phase B treatment groups after the 3^{rd} vaccination. In CMV-seropositive subjects, a single hCMV mRNA vaccine A vaccination boosted nAb GMT in a dose-related manner which further increased after the 2^{nd} vaccination to GMTs 10-fold to 19-fold over baseline across all hCMV mRNA vaccine A treatment groups, and up to 40-fold over baseline after the 3^{rd} vaccination in the Phase B hCMV mRNA vaccine A treatment groups.
c. Neutralizing antibody responses against fibroblast infection were dose-related and increased with subsequent vaccinations. In CMV-seronegative subjects, nAb GMTs generally met the natural infection benchmark after the 2^{nd} vaccination in the 90 μg, 180 μg, and 300 μg treatment groups and generally met or exceeded the natural infection benchmark after the 3^{rd} vaccination in all hCMV mRNA vaccine A treatment groups of Phase B. In CMV-seropositive subjects, a single hCMV mRNA vaccine A vaccination boosted GMTs in a dose-related manner and further boosted nAb titers to GMRs at least 2.5-fold over baseline across all hCMV mRNA vaccine A treatment groups the 2^{nd} vaccination and to GMRs at least 3.9-fold after the 3^{rd} vaccination in the Phase B hCMV mRNA vaccine A treatment groups.
d. Post-vaccination gB-specific T cell activation was observed across Phase B dose levels.
e. In Phase B CMV-seronegative participants at Month 12 (6 months after the 3^{rd} vaccination), GMTs of nAb against epithelial cell infection (a measure of immune response to intact CMV pentamer antigen) were 3.6-fold and 3.9-fold higher than the natural infection benchmark in the 90 μg and 180 μg treatment groups, and GMTs of nAb against fibroblast infection (a measure of immune response to CMV gB antigen) approximated the natural infection benchmark in the 90 μg and 180 μg treatment groups.
f. In Phase B CMV-seropositive participants at Month 12 (6 months after the 3^{rd} vaccination), GMRs of nAb against epithelial cell infection ranged 14-31 across the 30.tg, 90 μg and 180 μg treatment groups, and GMRs of nAb against fibroblast infection ranged 6-8.

### Sequences

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a polyA tail and/or cap (e.g., 7 mG(5′)ppp(5′)NlmpNp). Further, while some of the mRNAs and encoded antigen sequences described herein may include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 13)
GGGAAAUAAGAGAGAAAAGAAGAGUAAGAAGAAAUAUAAGAGCCACC
3′ UTR:
(SEQ ID NO: 14)
UGAUAAUAGGCUGGAGCCUCGGUGGCCAUGCUUCUUGCCCCUUGGGC
CUCCCCCCAGCCCCUCCUCCCCUUCCUGCACCCGUACCCCCGUGGUC
UUUGAAUAAAGUCUGAGUGGGCGGC

TABLE 5
hCMV mRNA and antigen sequences

| | | SEQ |
|---|---|---|
| | | ID |
| hCMV gB mRNA | | NO: |
| SEQ ID NO: 1 consists of, from 5′ end to 3′ end, 5′ UTR | | 1 |
| SEQ ID NO: 13, mRNA ORF SEQ ID NO: 7, and 3′ UTR SEQ ID NO: 14. | | |
| Chemistry | 1-methylpseudouridine | |
| Cap | 7mG(5′)ppp(′)NlmpNp | |
| 5′ UTR | GGGAAAUAAGAGAGAAAAGAAGAGUAAGAAGAAAUAUA | 13 |
| | AGAGCCACC | |
| ORF of mRNA | AUGGAAUCCAGGAUCUGGUGCCUGGUAGUCUGCGUUAAC | 7 |
| Construct | UUGUGUAUCGUCUGUCUGGGUGCUGCGGUUUCCUCAUCU | |
| (excluding | UCUACUCGUGGAACUUCUGCUACUCACAGUCACCAUUCC | |
| the stop | UCUCAUACGACGUCUGCUGCUCACUCUCGAUCCGGUUCA | |
| codon) | GUCUCUCAACGCGUAACUUCUUCCCAAACGGUCAGCCAU | |
| | GGUGUUAACGAGACCAUCUACAACACUACCCUCAAGUAC | |
| | GGAGAUGUGGUGGGGGUCAAUACCACCAAGUACCCCUAU | |
| | CGCGUGUGUUCUAUGGCCCAGGGUACGGAUCUUAUUCGC | |
| | UUUGAACGUAAUAUCGUCUGCACCUCGAUGAAGCCCAUC | |
| | AAUGAAGACCUGGACGAGGGCAUCAUGGUGGUCUACAAA | |
| | CGCAACAUCGUCGCGCACACCUUUAAGGUACGAGUCUAC | |
| | CAGAAGGUUUUGACGUUUCGUCGUAGCUACGCUUACAUC | |
| | CACACCACUUAUCUGCUGGGCAGCAACACGGAAUACGUG | |
| | GCGCCUCCUAUGUGGGAGAUUCAUCAUAUCAACAGCCAC | |
| | AGUCAGUGCUACAGUUCCUACAGCCGCGUUAUAGCAGGC | |
| | ACGGUUUUCGUGGCUUAUCAUAGGGACAGCUAUGAAAAC | |
| | AAAACCAUGCAAUUAAUGCCCGACGAUUAUUCCAACACC | |
| | CACAGUACCCGUUACGUGACGGUCAAGGAUCAAUGGCAC | |
| | AGCCGCGGCAGCACCUGGCUCUAUCGUGAGACCUGUAAU | |
| | CUGAAUUGUAUGGUGACCAUCACUACUGCGCGCUCCAAA | |
| | UAUCCUUAUCAUUUUUUCGCCACUUCCACGGGUGACGUG | |
| | GUUGACAUUUCUCCUUUCUACAACGGAACCAAUCGCAAU | |
| | GCCAGCUACUUUGGAGAAAACGCCGACAAGUUUUUCAUU | |
| | UUUCCGAACUACACUAUCGUCUCCGACUUUGGAAGACCG | |
| | AAUUCUGCGUUAGAGACCCACAGGUUGGUGGCUUUUCUU | |
| | GAACGUGCGGACUCGGUGAUCUCCUGGGAUAUACAGGAC | |
| | GAAAAGAAUGUCACUUGUCAACUCACUUUCUGGGAAGCC | |
| | UCGGAACGCACCAUUCGUUCCGAAGCCGAGGACUCGUAU | |
| | CACUUUUCUUCUGCCAAAAUGACCGCCACUUUCUUAUCU | |
| | AAGAAGCAAGAGGUGAACAUGUCCGACUCUGCGCUGGAC | |
| | UGCGUACGUGAUGAGGCUAUAAAUAAGUUACAGCAGAUU | |
| | UUCAAUACUUCAUACAAUCAAACAUAUGAAAAAUAUGGA | |
| | AACGUGUCCGUCUUUGAAACCACUGGUGGUUUGGUAGUG | |
| | UUCUGGCAAGGUAUCAAGCAAAAAUCUCUGGUGGAACUC | |
| | GAACGUUUGGCCAACCGCUCCAGUCUGAAUCUUACUCAU | |
| | AAUAGAACCAAAAGAAGUACAGAUGGCAACAAUGCAACU | |
| | CAUUUAUCCAACAUGGAAUCGGUGCACAAUCUGGUCUAC | |
| | GCCCAGCUGCAGUUCACCUAUGACACGUUGCGCGGUUAC | |
| | AUCAACCGGGCGCUGGCGCAAAUCGCAGAAGCCUGGUGU | |
| | GUGGAUCAACGGCGCACCCUAGAGGUCUUCAAGGAACUC | |
| | AGCAAGAUCAACCCGUCAGCCAUUCUCUCGGCCAUUUAC | |
| | AACAAACCGAUUGCCGCGCGUUUCAUGGGUGAUGUCUUG | |
| | GGCCUGGCCAGCUGCGUGACCAUCAACCAAACCAGCGUCA | |
| | AGGUGCUGCGUGAUAUGAACGUGAAGGAGUCGCCAGGAC | |
| | GCUGCUACUCACGACCCGUGGUCAUCUUUAAUUUCGCCA | |
| | ACAGCUCGUACGUGCAGUACGGUCAACUGGGCGAGGACA | |
| | ACGAAAUCCUGUUGGGCAACCACCGCACUGAGGAAUGUC | |
| | AGCUUCCCAGCCUCAAGAUCUUCAUCGCCGGGAACUCGGC | |
| | CCACGAGUACGUGGACUACCUCUUCAAACGCAUGAUUGA | |
| | CCUCAGCAGUAUCUCCACCGUCGACAGCAUGAUCGCCCUG | |
| | GAUAUCGACCCGCUGGAAAAUACCGACUUCAGGGUACUG | |
| | GAACUUUACUCGCAGAAAGAGCUGCGUUCCAGCAACGUU | |
| | UUUGACCUCGAAGAGAUCAUGCGCGAAUUCAACUCGUAC | |
| | AAGCAGCGGGUAAAGUACGUGGAGGACAAGGUAGUCGAC | |
| | CCGCUACCGCCCUACCUCAAGGGUCUGGACGACCUCAUGA | |
| | GCGGCCUGGGCGCCGCGGGAAAGGCCGUUGGCGUAGCCA | |
| | UUGGGGCCGUGGGUGGCGCGGUGGCCUCCGUGGUCGAAG | |
| | GCGUUGCCACCUUCCUCAAAAACCCCUUCGGAGCGUUCAC | |
| | CAUCAUCCUCGUGGCCAUAGCUGUAGUCAUUAUCACUUA | |
| | UUUGAUCUAUACUCGACAGCGGCGUUUGUGCACGCAGCC | |
| | GCUGCAGAACCUCUUUCCCUAUCUGGUGUCCGCCGACGG | |
| | GACCACCGUGACGUCGGGCAGCACCAAAGACACGUCGUU | |
| | ACAGGCUCCGCCUUCCUACGAGGAAAGUGUUUAUAAUUC | |
| | UGGUCGCAAAGGACCGGGACCACCGUCGUCUGAUGCAUC | |
| | CACGGCGGCUCCGCCUUACACCAACGAGCAGGCUUACCAG | |
| | AUGCUUCUGGCCCUGGCCCGUCUGGACGCAGAGCAGCGA | |
| | GCGCAGCAGAACGGUACAGAUUCUUUGGACGGACGGACU | |
| | GGCACGCAGGACAAGGGACAGAAGCCCAACCUACUAGAC | |
| | CGACUGCGACAUCGCAAAAACGGCUACCGACACUUGAAA | |
| | GACUCUGACGAAGAAGAGAACGUC | |
| 3′ UTR | UGAUAAUAGGCUGGAGCCUCGGUGGCCAUGCUUCUUGCC | 14 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MESRIWCLVVCVNLCIVCLGAAVSSSSTRGTSATHSHHSSHTT | 15 |
| amino acid | SAAHSRSGSVSQRVTSSQTVSHGVNETIYNTTLKYGDVVGVN | |
| sequence | TTKYPYRVCSMAQGTDLIRFERNIVCTSMKPINEDLDEGIMVV | |
| | YKRNIVAHTFKVRVYQKVLTFRRSYAYIHTTYLLGSNTEYVA | |
| | PPMWEIHHINSHSQCYSSYSRVIAGTVFVAYHRDSYENKTMQ | |
| | LMPDDYSNTHSTRYVTVKDQWHSRGSTWLYRETCNLNCMV | |
| | TITTARSKYPYHFFATSTGDVVDISPFYNGTNRNASYFGENAD | |
| | KFFIFPNYTIVSDFGRPNSALETHRLVAFLERADSVISWDIQDE | |
| | KNVTCQLTFWEASERTIRSEAEDSYHFSSAKMTATFLSKKQEV | |
| | NMSDSALDCVRDEAINKLQQIFNTSYNQTYEKYGNVSVFETT | |
| | GGLVVFWQGIKQKSLVELERLANRSSLNLTHNRTKRSTDGNN | |
| | ATHLSNMESVHNLVYAQLQFTYDTLRGYINRALAQIAEAWC | |
| | VDQRRTLEVFKELSKINPSAILSAIYNKPIAARFMGDVLGLASC | |
| | VTINQTSVKVLRDMNVKESPGRCYSRPVVIFNFANSSYVQYG | |
| | QLGEDNEILLGNHRTEECQLPSLKIFIAGNSAYEYVDYLFKRMI | |
| | DLSSISTVDSMIALDIDPLENTDFRVLELYSQKELRSSNVFDLE | |
| | EIMREFNSYKQRVKYVEDKVVDPLPPYLKGLDDLMSGLGAA | |
| | GKAVGVAIGAVGGAVASVVEGVATILKNPFGAITIILVAIAV | |
| | VIITYLIYTRQRRLCTQPLQNLFPYLVSADGTTVTSGSTKDTSL | |
| | QAPPSYEESVYNSGRKGPGPPSSDASTAAPPYTNEQAYQMLL | |
| | ALARLDAEQRAQQNGTDSLDGRTGTQDKGQKPNLLDRLRHR | |
| | KNGYRHLKDSDEEENV | |
| PolyA tail | 100 nt | |
| | | SEQ ID |
| hCMV UL128 | | NO: |
| SEQ ID NO: 2 consists of, from 5′ end to 3′ end, 5′ UTR | | 2 |
| SEQ ID NO: 13, mRNA ORF SEQ ID NO: 8, and 3′ UTR SEQ ID NO: 14. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 13 |
| | AGAGCCACC | |
| ORF of mRNA | AUGAGUCCCAAAGAUCUGACGCCGUUCUUGACGGCGUUG | 8 |
| Construct | UGGCUGCUAUUGGGUCACAGCCGCGUGCCGCGGGUGCGC | |
| (excluding | GCAGAAGAAUGUUGCGAAUUCAUAAACGUCAACCACCCG | |
| the stop | CCGGAACGCUGUUACGAUUUCAAAAUGUGCAAUCGCUUC | |
| codon) | ACCGUCGCGCUGCGGUGUCCGGACGGCGAAGUCUGCUAC | |
| | AGUCCCGAGAAAACGGCUGAGAUUCGCGGGAUCGUCACC | |
| | ACCAUGACCCAUUCAUUGACACGCCAGGUCGUACACAAC | |
| | AAACUGACGAGCUGCAACUACAAUCCGUUAUACCUCGAA | |
| | GCUGACGGGCGAAUACGCUGCGGCAAAGUAAACGACAAG | |
| | GCGCAGUACCUGCUGGGCGCCGCUGGCAGCGUUCCCUAUC | |
| | GAUGGAUCAAUCUGGAAUACGACAAGAUAACCCGGAUCG | |
| | UGGGCCUGGAUCAGUACCUGGAGAGCGUUAAGAAACACA | |
| | AACGGCUGGAUGUGUGCCGCGCUAAAAUGGGCUAUAUGC | |
| | UGCAG | |
| 3′ UTR | UGAUAAUAGGCUGGAGCCUCGGUGGCCAUGCUUCUUGCC | 14 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MSPKDLTPFLTALWLLLGHSRVPRVRAEECCEFINVNHPPERC | 16 |
| amino acid | YDFKMCNRFTVALRCPDGEVCYSPEKTAEIRGIVTTMTHSLTR | |
| sequence | QVVHNKLTSCNYNPLYLEADGRIRCGKVNDKAQYLLGAAGS | |
| | VPYRWINLEYDKITRIVGLDQYLESVKKHKRLDVCRAKMGY | |
| | MLQ | |
| PolyA tail | 100 nt | |
| | | SEQ ID |
| hCMV UL130 | | NO: |
| SEQ ID NO: 3 consists of, from 5′ end to 3′ end, 5′ UTR | | 3 |
| SEQ ID NO: 13, mRNA ORF SEQ ID NO: 9, and 3′ UTR SEQ ID NO: 14. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 13 |
| | AGAGCCACC | |
| ORF of mRNA | AUGCUGCGGCUUCUGCUUCGUCACCACUUUCACUGCCUGC | 9 |
| Construct | UUCUGUGCGCGGUUUGGGCAACGCCCUGUCUGGCGUCUC | |
| (excluding | CGUGGUCGACGCUAACAGCAAACCAGAAUCCGUCCCCGCC | |
| the stop | AUGGUCUAAACUGACGUAUUCCAAACCGCAUGACGCGGC | |
| codon) | GACGUUUUACUGUCCUUUUCUCUAUCCCUCGCCCCCACGA | |
| | UCCCCCUUGCAAUUCUCGGGGUUCCAGCGGGUAUCAACG | |
| | GGUCCCGAGUGUCGCAACGAGACCCUGUAUCUGCUGUAC | |
| | AACCGGGAAGGCCAGACCUUGGUGGAGAGAAGCUCCACC | |
| | UGGGUGAAAAAGGUGAUCUGGUACCUGAGCGGUCGGAAC | |
| | CAAACCAUCCUCCAACGGAUGCCCCGAACGGCUUCGAAAC | |
| | CGAGCGACGGAAACGUGCAGAUCAGCGUGGAAGACGCCA | |
| | AGAUUUUUGGAGCGCACAUGGUGCCCAAGCAGACCAAGC | |
| | UGCUACGCUUCGUCGUCAACGAUGGCACACGUUAUCAGA | |
| | UGUGUGUGAUGAAGCUGGAGAGCUGGGCUCACGUCUUCC | |
| | GGGACUACAGCGUGUCUUUUCAGGUGCGAUUGACGUUCA | |
| | CCGAGGCCAAUAACCAGACUUACACCUUCUGCACCCAUCC | |
| | CAAUCUCAUCGUU | |
| 3′ UTR | UGAUAAUAGGCUGGAGCCUCGGUGGCCAUGCUUCUUGCC | 14 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MLRLLLRHHFHCLLLCAVWATPCLASPWSTLTANQNPSPPWSK | 17 |
| amino acid | LTYSKPHDAATFYCPFLYPSPPRSPLQFSGFQRVSTGPECRNE | |
| sequence | TLYLLYNREGQTLVERSSTWVKKVIWYLSGRNQTILQRMPRT | |
| | ASKPSDGNVQ1SVEDAKIFGAHMVPKQTKLLRFVVNDGTRYQ | |
| | MCVMKLESWAHVFRDYSVSFQVRLTFTEANNQTYTFCTHPN | |
| | LIV | |
| PolyA tail | 100 nt | |
| | | SEQ ID |
| hCMV UL131 | | NO: |
| SEQ ID NO: 4 consists of. from 5′ end to 3′ end, 5′ UTR | | 4 |
| SEQ ID NO: 13, mRNA ORF SEQ ID NO: 10. and 3′ UTR SEQ ID NO: 14. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 13 |
| | AGAGCCACC | |
| ORF of mRNA | AUGCGGCUGUGUCGGGUGUGGCUGUCUGUUUGUCUGUGC | 10 |
| Construct | GCCGUGGUGCUGGGUCAGUGCCAGCGGGAAACCGCGGAA | |
| (excluding | AAGAACGAUUAUUACCGAGUACCGCAUUACUGGGACGCG | |
| the stop | UGCUCUCGCGCGCUGCCCGACCAAACCCGUUACAAGUAUG | |
| | UGGAACAGCUCGUGGACCUCACGUUGAACUACCACUACG | |
| | AUGCGAGCCACGGCUUGGACAACUUUGACGUGCUCAAGA | |
| | GAAUCAACGUGACCGAGGUGUCGUUGCUCAUCAGCGACU | |
| | UUAGACGUCAGAACCGUCGCGGCGGCACCAACAAAAGGA | |
| | CCACGUUCAACGCCGCCGGUUCGCUGGCGCCACACGCCCG | |
| | GAGCCUCGAGUUCAGCGUGCGGCUCUUUGCCAAC | |
| 3′ UTR | UGAUAAUAGGCUGGAGCCUCGGUGGCCAUGCUUCUUGCC | 14 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MRLCRVWLSVCLCAVVLGQCQRETAEKNDYYRVPHYWDAC | 18 |
| amino acid | SRALPDQTRYKYVEQLVDLTLNYHYDASHGLDNFDVLKRIN | |
| sequence | VTEVSLLISDFRRQNRRGGTNKRTTFNAAGSLAPHARSLEF | |
| | SVRLFAN | |
| PolyA tail | 100 nt | |
| | | SEQ ID |
| hCMV gH | | NO: |
| SEQ ID NO: 5 consists of, from 5′ end to 3′ end, 5′ UTR | | 5 |
| SEQ ID NO: 13, mRNA ORF SEQ ID NO: 11, and 3′ UTR SEQ ID NO: 14. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 13 |
| | AGAGCCACC | |
| ORF of mRNA | AUGCGGCCAGGCCUCCCCUCCUACCUCAUCAUCCUCGCCG | 11 |
| Construct | UCUGUCUCUUCAGCCACCUACUUUCGUCACGAUAUGGCG | |
| (excluding | CAGAAGCCGUAUCCGAACCGCUGGACAAAGCGUUUCACC | |
| the stop | UACUGCUCAACACCUACGGGAGACCCAUCCGCUUCCUGCG | |
| codon) | UGAAAAUACCACCCAGUGUACCUACAACAGCAGCCUCCG | |
| | UAACAGCACGGUCGUCAGGGAAAACGCCAUCAGUUUCAA | |
| | CUUCUUCCAAAGCUAUAAUCAAUACUAUGUAUUCCAUAU | |
| | GCCUCGAUGUCUCUUUGCGGGUCCUCUGGCGGAGCAGUU | |
| | UCUGAACCAGGUAGAUCUGACCGAAACCCUGGAAAGAUA | |
| | CCAACAGAGACUUAACACUUACGCGCUGGUAUCCAAAGA | |
| | CCUGGCCAGCUACCGAUCUUUCUCGCAGCAGCUAAAGGC | |
| | ACAAGACAGCCUAGGUGAACAGCCCACCACUGUGCCACCG | |
| | CCCAUUGACCUGUCAAUACCUCACGUUUGGAUGCCACCGC | |
| | AAACCACUCCACACGGCUGGACAGAAUCACAUACCACCUC | |
| | AGGACUACACCGACCACACUUUAACCAGACCUGUAUCCUC | |
| | UUUGAUGGACACGAUCUACUAUUCAGCACCGUCACACCU | |
| | UGUUUGCACCAAGGCUUUUACCUCAUCGACGAACUACGU | |
| | UACGUUAAAAUAACACUGACCGAGGACUUCUUCGUAGUU | |
| | ACGGUGUCCAUAGACGACGACACACCCAUGCUGCUUAUC | |
| | UUCGGCCAUCUUCCACGCGUACUUUUCAAAGCGCCCUAUC | |
| | AACGCGACAACUUUAUACUACGACAAACUGAGAAACACG | |
| | AGCUCCUGGUGCUAGUUAAGAAAGAUCAACUGAACCGUC | |
| | ACUCUUAUCUCAAAGACCCGGACUUUCUUGACGCCGCAC | |
| | UUGACUUCAACUACCUAGACCUCAGCGCACUACUACGUA | |
| | ACAGCUUUCACCGUUACGCCGUGGAUGUACUCAAGAGCG | |
| | GUCGAUGUCAGAUGCUGGACCGCCGCACGGUAGAAAUGG | |
| | CCUUCGCCUACGCAUUAGCACUGUUCGCAGCAGCCCGACA | |
| | AGAAGAGGCCGGCGCCCAAGUCUCCGUCCCACGGGCCCUA | |
| | GACCGCCAGGCCGCACUCUUACAAAUACAAGAAUUUAUG | |
| | AUCACCUGCCUCUCACAAACACCACCACGCACCACGUUGC | |
| | UGCUGUAUCCCACGGCCGUGGACCUGGCCAAACGAGCCCU | |
| | UUGGACACCGAAUCAGAUCACCGACAUCACCAGCCUCGU | |
| | ACGCCUGGUCUACAUACUCUCUAAACAGAAUCAGCAACA | |
| | UCUCAUCCCCCAAUGGGCACUACGACAGAUCGCCGACUUU | |
| | GCCCUAAAACUACACAAAACGCACCUGGCCUCUUUUCUU | |
| | UCAGCCUUCGCACGCCAAGAACUCUACCUCAUGGGCAGCC | |
| | UCGUCCACUCCAUGCUGGUACAUACGACGGAGAGACGCG | |
| | AAAUCUUCAUCGUAGAAACGGGCCUCUGUUCAUUGGCCG | |
| | AGCUAUCACACUUUACGCAGUUGUUAGCUCAUCCACACC | |
| | ACGAAUACCUCAGCGACCUGUACACACCCUGUUCCAGUA | |
| | GCGGGCGACGCGAUCACUCGCUCGAACGCCUCACGCGUCU | |
| | CUUCCCCGAUGCCACCGUCCCCGCUACCGUUCCCGCCGCC | |
| | CUCUCCAUCCUAUCUACCAUGCAACCAAGCACGCUGGAAA | |
| | CCUUCCCCGACCUGUUUUGCUUGCCGCUCGGCGAAUCCUU | |
| | CUCCGCGCUGACCGUCUCCGAACACGUCAGUUAUAUCGU | |
| | AACAAACCAGUACCUGAUCAAAGGUAUCUCCUACCCUGU | |
| | CUCCACCACCGUCGUAGGCCAGAGCCUCAUCAUCACCCAG | |
| | ACGGACAGUCAAACUAAAUGCGAACUGACGCGCAACAUG | |
| | CAUACCACACACAGCAUCACAGUGGCGCUCAACAUUUCGC | |
| | UAGAAAACUGCGCCUUUUGCCAAAGCGCCCUGCUAGAAU | |
| | ACGACGACACGCAAGGCGUCAUCAACAUCAUGUACAUGC | |
| | ACGACUCGGACGACGUCCUUUUCGCCCUGGAUCCCUACAA | |
| | CGAAGUGGUGGUCUCAUCUCCGCGAACUCACUACCUCAU | |
| | GCUUUUGAAGAACGGUACGGUACUAGAAGUAACUGACGU | |
| | CGUCGUGGACGCCACCGACAGUCGUCUCCUCAUGAUGUCC | |
| | GUCUACGCGCUAUCGGCCAUCAUCGGCAUCUAUCUGCUC | |
| | UACCGCAUGCUCAAGACAUGC | |
| 3′ UTR | UGAUAAUAGGCUGGAGCCUCGGUGGCCAUGCUUCUUGCC | 14 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MRPGLPSYLIILAVCLFSHLLSSRYGAEAVSEPLDKAFHLLLNT | 19 |
| amino acid | YGRPIRFLRENTTQCTYNSSLRNSTVVRENAISFNFFQSYNQY | |
| sequence | YVFHMPRCLFAGPLAEQFLNQVDLTETLERYQQRLNTYALVS | |
| | KDLASYRSFSQQLKAQDSLGEQPTTVPPPIDLSIPHVWMPPQT | |
| | TPHGWTESHTTSGLHRPHFWQTCILFDGHDLLFSTVTPCLHQG | |
| | FYLIDELRYVKITLTEDFFVVTVSIDDDTPMLLIFGHLPRVLFK | |
| | APYQRDNFILRQTEKHELLVLVKKDQLNRHSYLKDPDFLDAA | |
| | LDFNYLDLSALLRNSFHRYAVDVLKSGRCQMLDRRTVEMAF | |
| | AYALALFAAARQEEAGAQVSVPRALDRQAALLQIQEFMITCLS | |
| | QTPPRTTLLLYPTAVDLAKRALWTPNQITDITSLVRLVYILSK | |
| | QNQQHLIPQWALRQIADFALKLHKTHLASFLSAFARQELYLM | |
| | GSLVHSMLVHTTERREIFIVETGLCSLAELSHFTQLLAHPHHEY | |
| | LSDLYTPCSSSGRRDHSLERLTRLFPDATVPATVPAALSILSTM | |
| | QPSTLETFPDLFCLPLGESFSALTVSEHVSYIVTNQYLIKGISY | |
| | PVSTTVVGQSLIITQTDSQTKCELTRNMHTTHSITVALNISLEN | |
| | ACFCQSALLEYDDTQGVINIMYMHDSDDVLFALDPYNEVVVSS | |
| | PRTHYLMLLKNGTVLEVTDVVVDATDSRLLMMSVYALSAIIG | |
| | IYLLYRMLKTC | |
| PolyA tail | 100 nt | |
| | | SEQ ID |
| hCMV gL | | NO: |
| SEQ ID NO: 6 consists of, from 5′ end to 3′ end, 5′ UTR | | 6 |
| SEQ ID NO: 13. mRNA ORF SEQ ID NO: 12. and 3′ UTR SEQ ID NO: 14. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 13 |
| | AGAGCCACC | |
| ORF of mRNA | AUGUGCCGCCGCCCGGAUUGCGGCUUCUCUUUCUCACCUG | 12 |
| Construct | GACCGGUGAUACUGCUGUGGUGUUGCCUUCUGCUGCCCA | |
| (excluding | UUGUUUCCUCAGCCGCCGUCAGCGUCGCUCCUACCGCCGC | |
| the stop | CGAGAAAGUCCCCGCGGAGUGCCCCGAACUAACGCGCCGA | |
| codon) | UGCUUGUUGGGUGAGGUGUUUGAGGGUGACAAGUAUGAA | |
| | AGUUGGCUGCGCCCGUUGGUGAAUGUUACCGGGCGCGAU | |
| | GGCCCGCUAUCGCAACUUAUCCGUUACCGUCCCGUUACGC | |
| | CGGAGGCCGCCAACUCCGUGCUGUUGGACGAGGCUUUCC | |
| | UGGACACUCUGGCCCUGCUGUACAACAAUCCGGAUCAAU | |
| | UGCGGGCCCUGCUGACGCUGUUGAGCUCGGACACAGCGC | |
| | CGCGCUGGAUGACGGUGAUGCGCGGCUACAGCGAGUGCG | |
| | GCGAUGGCUCGCCGGCCGUGUACACGUGCGUGGACGACC | |
| | UGUGCCGCGGCUACGACCUCACGCGACUGUCAUACGGGC | |
| | GCAGCAUCUUCACGGAACACGUGUUAGGCUUCGAGCUGG | |
| | UGCCACCGUCUCUCUUUAACGUGGUGGUGGCCAUACGCA | |
| | ACGAAGCCACGCGUACCAACCGCGCCGUGCGUCUGCCCGU | |
| | GAGCACCGCUGCCGCGCCCGAGGGCAUCACGCUCUUUUAC | |
| | GGCCUGUACAACGCAGUGAAGGAAUUCUGCCUGCGUCAC | |
| | CAGCUGGACCCGCCGCUGCUACGCCACCUAGAUAAAUACU | |
| | ACGCCGGACUGCCGCCCGAGCUGAAGCAGACGCGCGUCAA | |
| | CCUGCCGGCUCACUCGCGCUAUGGCCCUCAAGCAGUGGAU | |
| | GCUCGC | |
| 3′ UTR | UGAUAAUAGGCUGGAGCCUCGGUGGCCAUGCUUCUUGCC | 14 |
| | CCUUGGGCCUCCCCCCAGCCCCUCCUCCCCUUCCUGCACC | |
| | CGUACCCCCGUGGUCUUUGAAU AAAGUCUGAGUGGGCGG | |
| | C | |
| Corresponding | MCRRPDCGFSFSPGPVILLWCCLLLPIVSSAAVSVAPTAAEKV | 20 |
| amino acid | PAECPELTRRCLLGEVFEGDKYESWLRPLVNVTGRDGPLSQLI | |
| sequence | RYRPVTPEAANSVLLLDEAFLDTLALLLYNNPDQLRALLTLLLS | |
| | SDTAPRWMTVMRGYSECGDGSPAVYTCVDDLCRGYDLTRLSYG | |
| | RSIFTEHVLGFELVPPSLFNVVVAIRNEATRTNRAVRLPVSTAA | |
| | APEGITLFYGLYNAVKEFCLRHQLDPPLLRHLDKYYAGLPPEL | |
| | KQTRVNLPAHSRYGPQAVDAR | |
| PolyA tail | 100 nt | |
| | | SEQ ID |
| hCMV pp65mut | | NO: |
| SEQ ID NO: 21 consists of from 5′ end to 3′ end, 5′ UTR | | 21 |
| SEQ ID NO: 13, mRNA ORE SEQ ID NO: 22, and 3′ UTR SEQ ID NO: 14. | | |
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUA | 13 |
| | AGAGCCACC | |
| ORF of mRNA | AUGGAGUCGCGCGGUCGCCGUUGUCCCGAAAUGAUAUCC | 23 |
| Construct | GUACUGGGUCCCAUUUCGGGGCACGUGCUGAAAGCCGUG | |
| (excluding | UUUAGUCGCGGCGAUACGCCGGUGCUGCCGCACGAGACG | |
| the stop | AGACUCCUGCAGACGGGUAUCCACGUACGCGUGAGCCAG | |
| codon) | CCCUCGCUGAUCCUGGUGUCGCAGUACACGCCCGACUCGA | |
| | CGCCAUGCCACCGCGGCGACAAUCAGCUGCAGGUGCAGCA | |
| | CACGUACUUUACGGGCAGCGAGGUGGAGAACGUGUCGGU | |
| | CAACGUGCACAACCCCACGGGCCGAAGCAUCUGCCCCAGC | |
| | CAAGAGCCCAUGUCGAUCUAUGUGUACGCGCUGCCGCUC | |
| | AAGAUGCUGAACAUCCCCAGCAUCAACGUGCACCACUACC | |
| | CGUCGGCGGCCGAGCGCAAACACCGACACCUGCCCGUAGC | |
| | CGACGCUGUUAUUCACGCGUCGGGCAAGCAGAUGUGGCA | |
| | GGCGCGUCUCACGGUCUCGGGACUGGCCUGGACGCGUCA | |
| | GCAGAACCAGUGGAAAGAGCCCGACGUCUACUACACGUC | |
| | AGCGUUCGUGUUUCCCACCAAGGACGUGGCACUGCGGCA | |
| | CGUGGUGUGCGCGCACGAGCUGGUUUGCUCCAUGGAGAA | |
| | CACGCGCGCAACCAAGAUGCAGGUGAUAGGUGACCAGUA | |
| | CGUCAAGGUGUACCUGGAGUCCUUCUGCGAGGACGUGCC | |
| | CUCCGGCAAGCUCUUUAUGCACGUCACGCUGGGCUCUGA | |
| | CGUGGAAGAGGACCUAACGAUGACCCGCAACCCGCAACCC | |
| | UUCAUGCGCCCCCACGAGCGCAACGGCUUUACGGUGUUG | |
| | UGUCCCAAAAAUAUGAUAAUCAAACCGGGCAAGAUCUCG | |
| | CACAUCAUGCUGGAUGUGGCUUUUACCUCACACGAGCAU | |
| | UUUGGGCUGCUGUGUCCCAAGAGCAUCCCGGGCCUGAGC | |
| | AUCUCAGGUAACCUGUUGAUGAACGGGCAGCAAAUCUUC | |
| | CUGGAGGUACAAGCGAUACGCGAGACCGUGGAACUGCGU | |
| | CAGUACGAUCCCGUGGCUGCGCUCUUCUUUUUCGAUAUC | |
| | GACUUGUUGCUGCAGCGCGGGCCUCAGUACAGCGAGCAC | |
| | CCCACCUUCACCAGCCAGUAUCGCAUCCAGGGCAAGCUUG | |
| | AGUACCGACACACCUGGGACCGGCACGACGAGGGUGCCG | |
| | CCCAGGGCGACGACGACGUCUGGACCAGCGGAUCGGACU | |
| | CCGACGAAGAACUCGUAACCACCGAGCGUAAGACGCCCCG | |
| | CGUCACCGGCGGCGGAGCCAUGGCGAGCGCCUCCACUUCC | |
| | GCGGGCUCAGCAUCCUCGGCGACGGCGUGCACGGCGGGC | |
| | GUUAUGACACGCGGCCGCCUUAAGGCCGAGUCCACCGUC | |
| | GCGCCCGAAGAGGACACCGACGAGGAUUCCGACAACGAA | |
| | AUCCACAAUCCGGCCGUGUUCACCUGGCCGCCCUGGCAGG | |
| | CCGGCAUCCUGGCCCGCAACCUGGUGCCCAUGGUGGCUAC | |
| | GGUUCAGGGUCAGAAUCUGAAGUACCAGGAGUUCUUCUG | |
| | GGACGCCAACGACAUCUACCGCAUCUUCGCCGAAUUGGA | |
| | AGGCGUAUGGCAGCCCGCUGCGCAACCCAAACGUCGCCGC | |
| | CACCGGCAAGACGCCUUGCCCGGGCCAUGCAUCGCCUCGA | |
| | CGCCCAAAAAGCACCGAGGU | |
| 3′ UTR | UGAUAAUAGGCUGGAGCCUCGGUGGCCAUGCUUCUUGCC | 14 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding | MESRGRRCPEMISVLGPISGHVLKAVFSRGDTPVLPHETRLLQ | 24 |
| amino acid | TGIHVRVSQPSLILVSQYTPDSTPCHRGDNQLQVQHTYFTGSE | |
| sequence | VENVSVNVHNPTGRSICPSQEPMSIYVYALPLKMLNIPSINVH | |

### Equivalents

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

What is claimed is:

1. A method for producing an antigen-specific immune response to human cytomegalovirus (hCMV) in a human subject comprising:
administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle,
thereby inducing an antigen-specific immune response to hCMV in the subject, wherein the geometric mean titer (GMT) of neutralizing antibodies against epithelial cell infection increases in the human subject at least 3-fold relative to baseline following administration of the immunogenic composition.

2. A method for producing an antigen-specific immune response to human cytomegalovirus (hCMV) in a human subject comprising:
administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle,
thereby inducing an antigen-specific immune response to hCMV in the subject, wherein the geometric mean ratio (GMR) of neutralizing antibodies against epithelial cell infection in the human subject is about 9-41 following administration of the immunogenic composition.

3. A method for producing an antigen-specific immune response to human cytomegalovirus (hCMV) in a human subject comprising:
administering to a human subject a 30 μg to 200 μg dose of an immunogenic composition comprising (a) a messenger ribonucleic acid (mRNA) polynucleotide comprising an open reading frame encoding a hCMV gH polypeptide; (b) a mRNA polynucleotide comprising an open reading frame encoding a hCMV gL polypeptide; (c) a mRNA polynucleotide comprising an open reading frame encoding a hCMV UL128 polypeptide; (d) a mRNA polynucleotide comprising an open reading frame encoding a hCMV UL130 polypeptide; (e) a mRNA polynucleotide comprising an open reading frame encoding a hCMV UL131A polypeptide; and (f) a mRNA polynucleotide comprising an open reading frame encoding a hCMV gB polypeptide, wherein the mRNA polynucleotides of (a)-(f) are formulated in at least one lipid nanoparticle,
thereby inducing an antigen-specific immune response to hCMV in the subject, wherein the geometric mean ratio (GMR) of neutralizing antibodies against fibroblast infection in the human subject is about 4-8 following administration of the immunogenic composition.

4. The method of any one of claims 1-3, wherein the immunogenic composition is administered at a dose of 30 μg.

5. The method of any one of claims 1-3, wherein the immunogenic composition is administered at a dose of 90 μg.

6. The method of any one of claims 1-3, wherein the immunogenic composition is administered at a dose of 180 μg.

7. The method of any one of claims 1-3, wherein the immunogenic composition is administered at a dose of 300 μg.

8. The method of any one of claims 1-7, wherein at least two doses or at least three doses of the immunogenic composition are administered.

9. The method of claim 8, wherein three doses of the immunogenic composition are administered.

10. The method of claim 9, wherein doses of the immunogenic composition are administered on: Day 1; around the beginning of month 2; and around the beginning of month 6.

11. The method of any one of claims 1-10, wherein administration of a single dose of the immunogenic composition elicits serum neutralizing antibody titers against hCMV.

12. The method of claim 1, wherein the GMT of neutralizing antibodies against epithelial cell infection increases in the human subject at least 3-fold relative to baseline following a single dose, following two doses, or following three doses of the immunogenic composition.

13. The method of claim 12, wherein the GMT of neutralizing antibodies against epithelial cell infection increases in the human subject at least 3-fold relative to baseline following two doses or following three doses of the immunogenic composition.

14. The method of claim 12, wherein the GMT of neutralizing antibodies against epithelial cell infection increases in the human subject 9-20 fold relative to baseline following two doses of the vaccine composition.

15. The method of claim 12, wherein the GMT of neutralizing antibodies against epithelial cell infection increases in the subject 20-40-fold relative to baseline following three doses of the vaccine composition.

16. The method of claim 2, wherein the GMR of neutralizing antibodies against epithelial cell infection in seropositive subjects administered at least 2 doses of ≥30 μg of the immunogenic composition is in the range of 14-26.

17. The method of claim 2, wherein the GMR of neutralizing antibodies against epithelial cell infection in seropositive subjects administered at least 3 doses of ≥30 μg of the immunogenic composition is in the range of 14-26. 2

18. The method of claim 2, wherein the GMR of neutralizing antibodies against epithelial cell infection in seropositive subjects administered at least 3 doses of ≥30 μg of the immunogenic composition is in the range of 14-41.

19. The method of claim 18, wherein the GMR is in the range of 30-41.

20. The method of claim 19, wherein at least 3 doses of about 180 μg are administered to the seropositive subjects.

21. The method of any one of claims 1-20, wherein the lipid nanoparticle comprises: an ionizable cationic lipid; cholesterol; 1,2-distearoyl-sn-glycero-3-phosphocholine (DSPC); and 1,2 dimyristoyl-sn-glycerol, methoxypolyethyleneglycol (DMG-PEG).

22. The method of claim 21, wherein the ionizable cationic lipid comprises Compound I:

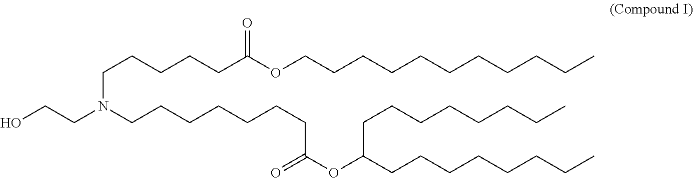 [SMILES: CCCCCCCCCCCOC(=O)CCCCCN(CCO)CCCCCCCC(=O)OC(CCCCCCCC)CCCCCCCC]

23. The method of any one of claims 1-22, wherein the lipid nanoparticle comprises a mixture of lipids comprising 20-60 mol % ionizable cationic lipid, 25-55 mol % cholesterol, 5-25 mol % DSPC, and 0.5-15 mol % DMG-PEG.

24. The method of claim 23, wherein the lipid nanoparticle comprises a mixture of lipids comprising 45-55 mol % ionizable cationic lipid, 35-40 mol % cholesterol, 5-15 mol % DSPC, and 1-2 mol % DMG-PEG.

25. The method of claim 24, wherein the lipid nanoparticle comprises a mixture of lipids comprising 50 mol % ionizable cationic lipid, 38.5 mol % cholesterol, 10 mol % DSPC, and 1.5 mol % DMG-PEG.

26. The method of any one of claims 1-25, wherein the weight ratio of the mRNA encoding hCMV gH, gL, UL128, UL130, UL131A, and gB proteins in the vaccine composition is 1:1:1:1:1:1.

27. The method of any one of claims 1-26, wherein the mRNA encoding hCMV gH, gL, UL128, UL130, UL131A, and gB proteins comprise a 1-methylpseudourine chemical modification.

28. The method of any one of claims 1-27, wherein the mRNA encoding hCMV gH protein comprises a nucleotide sequence having at least 90% identity to the sequence of SEQ ID NO: 5.

29. The method of claim 28, wherein the mRNA encoding hCMV gH protein comprises the nucleotide sequence of sequence of SEQ ID NO: 5.

30. The method of any one of claims 1-29, wherein the mRNA encoding hCMV gL protein comprises a nucleotide sequence having at least 90% identity to the sequence of SEQ ID NO: 6.

31. The method of claim 30, wherein the mRNA encoding hCMV gL protein comprises the nucleotide sequence of sequence of SEQ ID NO: 6.

32. The method of any one of claims 1-31, wherein the mRNA encoding hCMV UL128 protein comprises a nucleotide sequence having at least 90% identity to the sequence of SEQ ID NO: 2.

33. The method of claim 32, wherein the mRNA encoding hCMV UL128 protein comprises the nucleotide sequence of sequence of SEQ ID NO: 2.

34. The method of any one of claims 1-33, wherein the mRNA encoding hCMV UL130 protein comprises a nucleotide sequence having at least 90% identity to the sequence of SEQ ID NO: 3.

35. The method of claim 34, wherein the mRNA encoding hCMV UL130 protein comprises the nucleotide sequence of sequence of SEQ ID NO: 3.

36. The method of any one of claims 1-35, wherein the mRNA encoding hCMV UL131A protein comprises a nucleotide sequence having at least 90% identity to the sequence of SEQ ID NO: 4.

37. The method of claim 36, wherein the mRNA encoding hCMV UL131A protein comprises the nucleotide sequence of sequence of SEQ ID NO: 4.

38. The method of any one of claims 1-37, wherein the mRNA encoding hCMV gB protein comprises a nucleotide sequence having at least 90% identity to the sequence of SEQ ID NO: 1.

39. The method of claim 38, wherein the mRNA encoding hCMV gB protein comprises the nucleotide sequence of sequence of SEQ ID NO: 1.

40. The method of any one of claims 1-39, wherein the mRNA encoding hCMV gH protein comprises the nucleotide sequence of sequence of SEQ ID NO: 5, the mRNA encoding hCMV gL protein comprises the nucleotide sequence of sequence of SEQ ID NO: 6, the mRNA encoding hCMV UL128 protein comprises the nucleotide sequence of sequence of SEQ ID NO: 2, the mRNA encoding hCMV UL130 protein comprises the nucleotide sequence of sequence of SEQ ID NO: 3, the mRNA encoding hCMV UL131A protein comprises the nucleotide sequence of sequence of SEQ ID NO: 4, and the mRNA encoding hCMV gB protein comprises the nucleotide sequence of sequence of SEQ ID NO: 1.

41. The method of any one of claims 1-40, wherein the open reading frame encoding the hCMV gH polypeptide comprises a sequence having at least 90% identity to the sequence of SEQ ID NO: 11, the open reading frame encoding the hCMV gL polypeptide comprises a sequence having at least 90% identity to the sequence of SEQ ID NO: 12, the open reading frame encoding the hCMV UL128 polypeptide comprises a sequence having at least 90% identity to the sequence of SEQ ID NO: 8, the open reading frame encoding the hCMV UL130 polypeptide comprises a sequence having at least 90% identity to the sequence of SEQ ID NO: 9, the open reading frame encoding the hCMV UL131A polypeptide comprises a sequence having at least 90% identity to the of sequence of SEQ ID NO: 10, and/or the open reading frame encoding the hCMV gB polypeptide comprises a sequence having at least 90% identity to the sequence of SEQ ID NO: 7.

42. The method of any one of claims 1-41, wherein the immunogenic composition is administered via intramuscular injection.

43. The method of any one of claims 1-42, wherein the human subject is CMV-seropositive prior to being administered the hCMV mRNA immunogenic composition.

44. The method of any one of claims 1-42, wherein the human subject is CMV-seronegative prior to being administered the hCMV mRNA immunogenic composition.

45. The method of any one of claims 1-44, further comprising administering to a human subject a dose of 5 μg to 100 μg of a second immunogenic composition comprising at least one messenger ribonucleic acid (mRNA) polynucleotide comprising an open reading frame encoding a hCMV pp65 polypeptide, wherein the mRNA polynucleotide is formulated in at least one lipid nanoparticle.

46. The method of claim 45, wherein the second immunogenic composition is administered at a dose of 10 μg.

47. The method of claim 45, wherein the second immunogenic composition is administered ata dose of 40 μg.

48. The method of claim 45, wherein the second immunogenic composition is administered at a dose of 80 μg.

49. The method of any one of claims 45-48, wherein the mRNA encoding hCMV pp65 protein comprises a nucleotide sequence having at least 90% identity to the sequence of SEQ ID NO: 21.

50. The method of claim 49, wherein the mRNA encoding hCMV pp65 protein comprises the nucleotide sequence of sequence of SEQ ID NO: 21.

51. The method of any one of claims 45-48, wherein the open reading frame encoding the hCMV pp65 polypeptide comprises a sequence having at least 90% identity to the sequence of SEQ ID NO: 23.

52. The method of any one of claims 45-51, wherein the second vaccine composition is administered via intramuscular injection.

53. The method of claim 41, wherein the open reading frame encoding the hCMV gH polypeptide comprises SEQ ID NO: 11, the open reading frame encoding the hCMV gL polypeptide comprises SEQ ID NO: 12, the open reading frame encoding the hCMV UL128 polypeptide comprises SEQ ID NO: 8, the open reading frame encoding the hCMV UL130 polypeptide comprises SEQ ID NO: 9, the open reading frame encoding the hCMV UL131A polypeptide comprises SEQ ID NO: 10, and/or the open reading frame encoding the hCMV gB polypeptide comprises the sequence of SEQ ID NO: 7.

54. The method of claim 51, wherein the open reading frame encoding the hCMV pp65 polypeptide comprises SEQ ID NO: 23.


SEQUENCE LISTING (32 sequences)

SEQ ID NO: 1 - RNA, 2887 nt, Artificial Sequence
  Synthetic
gggaaauaagagagaaaagaagaguaagaagaaauauaagagccaccauggaauccaggaucuggugccugguagucugcguuaacuuguguaucgucugucugggugcugcgguuuccucaucuucuacucguggaacuucugcuacucacagucaccauuccucucauacgacgucugcugcucacucucgauccgguucagucucucaacgcguaacuucuucccaaacggucagccaugguguuaacgagaccaucuacaacacuacccucaaguacggagauguggugggggucaauaccaccaaguaccccuaucgcguguguucuauggcccaggguacggaucuuauucgcuuugaacguaauaucgucugcaccucgaugaagcccaucaaugaagaccuggacgagggcaucaugguggucuacaaacgcaacaucgucgcgcacaccuuuaagguacgagucuaccagaagguuuugacguuucgucguagcuacgcuuacauccacaccacuuaucugcugggcagcaacacggaauacguggcgccuccuaugugggagauucaucauaucaacagccacagucagugcuacaguuccuacagccgcguuauagcaggcacgguuuucguggcuuaucauagggacagcuaugaaaacaaaaccaugcaauuaaugcccgacgauuauuccaacacccacaguacccguuacgugacggucaaggaucaauggcacagccgcggcagcaccuggcucuaucgugagaccuguaaucugaauuguauggugaccaucacuacugcgcgcuccaaauauccuuaucauuuuuucgccacuuccacgggugacgugguugacauuucuccuuucuacaacggaaccaaucgcaaugccagcuacuuuggagaaaacgccgacaaguuuuucauuuuuccgaacuacacuaucgucuccgacuuuggaagaccgaauucugcguuagagacccacagguugguggcuuuucuugaacgugcggacucggugaucuccugggauauacaggacgaaaagaaugucacuugucaacucacuuucugggaagccucggaacgcaccauucguuccgaagccgaggacucguaucacuuuucuucugccaaaaugaccgccacuuucuuaucuaagaagcaagaggugaacauguccgacucugcgcuggacugcguacgugaugaggcuauaaauaaguuacagcagauuuucaauacuucauacaaucaaacauaugaaaaauauggaaacguguccgucuuugaaaccacuggugguuugguaguguucuggcaagguaucaagcaaaaaucucugguggaacucgaacguuuggccaaccgcuccagucugaaucuuacucauaauagaaccaaaagaaguacagauggcaacaaugcaacucauuuauccaacauggaaucggugcacaaucuggucuacgcccagcugcaguucaccuaugacacguugcgcgguuacaucaaccgggcgcuggcgcaaaucgcagaagccugguguguggaucaacggcgcacccuagaggucuucaaggaacucagcaagaucaacccgucagccauucucucggccauuuacaacaaaccgauugccgcgcguuucaugggugaugucuugggccuggccagcugcgugaccaucaaccaaaccagcgucaaggugcugcgugauaugaacgugaaggagucgccaggacgcugcuacucacgacccguggucaucuuuaauuucgccaacagcucguacgugcaguacggucaacugggcgaggacaacgaaauccuguugggcaaccaccgcacugaggaaugucagcuucccagccucaagaucuucaucgccgggaacucggccuacgaguacguggacuaccucuucaaacgcaugauugaccucagcaguaucuccaccgucgacagcaugaucgcccuggauaucgacccgcuggaaaauaccgacuucaggguacuggaacuuuacucgcagaaagagcugcguuccagcaacguuuuugaccucgaagagaucaugcgcgaauucaacucguacaagcagcggguaaaguacguggaggacaagguagucgacccgcuaccgcccuaccucaagggucuggacgaccucaugagcggccugggcgccgcgggaaaggccguuggcguagccauuggggccguggguggcgcgguggccuccguggucgaaggcguugccaccuuccucaaaaaccccuucggagcguucaccaucauccucguggccauagcuguagucauuaucacuuauuugaucuauacucgacagcggcguuugugcacgcagccgcugcagaaccucuuucccuaucugguguccgccgacgggaccaccgugacgucgggcagcaccaaagacacgucguuacaggcuccgccuuccuacgaggaaaguguuuauaauucuggucgcaaaggaccgggaccaccgucgucugaugcauccacggcggcuccgccuuacaccaacgagcaggcuuaccagaugcuucuggcccuggcccgucuggacgcagagcagcgagcgcagcagaacgguacagauucuuuggacggacggacuggcacgcaggacaagggacagaagcccaaccuacuagaccgacugcgacaucgcaaaaacggcuaccgacacuugaaagacucugacgaagaagagaacgucugauaauaggcuggagccucgguggccaugcuucuugccccuugggccuccccccagccccuccuccccuuccugcacccguacccccguggucuuugaauaaagucugagugggcggc

SEQ ID NO: 2 - RNA, 679 nt, Artificial Sequence
  Synthetic
gggaaauaagagagaaaagaagaguaagaagaaauauaagagccaccaugagucccaaagaucugacgccguucuugacggcguuguggcugcuauugggucacagccgcgugccgcgggugcgcgcagaagaauguugcgaauucauaaacgucaaccacccgccggaacgcuguuacgauuucaaaaugugcaaucgcuucaccgucgcgcugcgguguccggacggcgaagucugcuacagucccgagaaaacggcugagauucgcgggaucgucaccaccaugacccauucauugacacgccaggucguacacaacaaacugacgagcugcaacuacaauccguuauaccucgaagcugacgggcgaauacgcugcggcaaaguaaacgacaaggcgcaguaccugcugggcgccgcuggcagcguucccuaucgauggaucaaucuggaauacgacaagauaacccggaucgugggccuggaucaguaccuggagagcguuaagaaacacaaacggcuggaugugugccgcgcuaaaaugggcuauaugcugcagugauaauaggcuggagccucgguggccaugcuucuugccccuugggccuccccccagccccuccuccccuuccugcacccguacccccguggucuuugaauaaagucugagugggcggc

SEQ ID NO: 3 - RNA, 808 nt, Artificial Sequence
  Synthetic
gggaaauaagagagaaaagaagaguaagaagaaauauaagagccaccaugcugcggcuucugcuucgucaccacuuucacugccugcuucugugcgcgguuugggcaacgcccugucuggcgucuccguggucgacgcuaacagcaaaccagaauccguccccgccauggucuaaacugacguauuccaaaccgcaugacgcggcgacguuuuacuguccuuuucucuaucccucgcccccacgaucccccuugcaauucucgggguuccagcggguaucaacgggucccgagugucgcaacgagacccuguaucugcuguacaaccgggaaggccagaccuugguggagagaagcuccaccugggugaaaaaggugaucugguaccugagcggucggaaccaaaccauccuccaacggaugccccgaacggcuucgaaaccgagcgacggaaacgugcagaucagcguggaagacgccaagauuuuuggagcgcacauggugcccaagcagaccaagcugcuacgcuucgucgucaacgauggcacacguuaucagaugugugugaugaagcuggagagcugggcucacgucuuccgggacuacagcgugucuuuucaggugcgauugacguucaccgaggccaauaaccagacuuacaccuucugcacccaucccaaucucaucguuugauaauaggcuggagccucgguggccaugcuucuugccccuugggccuccccccagccccuccuccccuuccugcacccguacccccguggucuuugaauaaagucugagugggcggc

SEQ ID NO: 4 - RNA, 553 nt, Artificial Sequence
  Synthetic
gggaaauaagagagaaaagaagaguaagaagaaauauaagagccaccaugcggcugugucggguguggcugucuguuugucugugcgccguggugcugggucagugccagcgggaaaccgcggaaaagaacgauuauuaccgaguaccgcauuacugggacgcgugcucucgcgcgcugcccgaccaaacccguuacaaguauguggaacagcucguggaccucacguugaacuaccacuacgaugcgagccacggcuuggacaacuuugacgugcucaagagaaucaacgugaccgaggugucguugcucaucagcgacuuuagacgucagaaccgucgcggcggcaccaacaaaaggaccacguucaacgccgccgguucgcuggcgccacacgcccggagccucgaguucagcgugcggcucuuugccaacugauaauaggcuggagccucgguggccaugcuucuugccccuugggccuccccccagccccuccuccccuuccugcacccguacccccguggucuuugaauaaagucugagugggcggc

SEQ ID NO: 5 - RNA, 2392 nt, Artificial Sequence
  Synthetic
gggaaauaagagagaaaagaagaguaagaagaaauauaagagccaccaugcggccaggccuccccuccuaccucaucauccucgccgucugucucuucagccaccuacuuucgucacgauauggcgcagaagccguauccgaaccgcuggacaaagcguuucaccuacugcucaacaccuacgggagacccauccgcuuccugcgugaaaauaccacccaguguaccuacaacagcagccuccguaacagcacggucgucagggaaaacgccaucaguuucaacuucuuccaaagcuauaaucaauacuauguauuccauaugccucgaugucucuuugcggguccucuggcggagcaguuucugaaccagguagaucugaccgaaacccuggaaagauaccaacagagacuuaacacuuacgcgcugguauccaaagaccuggccagcuaccgaucuuucucgcagcagcuaaaggcacaagacagccuaggugaacagcccaccacugugccaccgcccauugaccugucaauaccucacguuuggaugccaccgcaaaccacuccacacggcuggacagaaucacauaccaccucaggacuacaccgaccacacuuuaaccagaccuguauccucuuugauggacacgaucuacuauucagcaccgucacaccuuguuugcaccaaggcuuuuaccucaucgacgaacuacguuacguuaaaauaacacugaccgaggacuucuucguaguuacgguguccauagacgacgacacacccaugcugcuuaucuucggccaucuuccacgcguacuuuucaaagcgcccuaucaacgcgacaacuuuauacuacgacaaacugagaaacacgagcuccuggugcuaguuaagaaagaucaacugaaccgucacucuuaucucaaagacccggacuuucuugacgccgcacuugacuucaacuaccuagaccucagcgcacuacuacguaacagcuuucaccguuacgccguggauguacucaagagcggucgaugucagaugcuggaccgccgcacgguagaaauggccuucgccuacgcauuagcacuguucgcagcagcccgacaagaagaggccggcgcccaagucuccgucccacgggcccuagaccgccaggccgcacucuuacaaauacaagaauuuaugaucaccugccucucacaaacaccaccacgcaccacguugcugcuguaucccacggccguggaccuggccaaacgagcccuuuggacaccgaaucagaucaccgacaucaccagccucguacgccuggucuacauacucucuaaacagaaucagcaacaucucaucccccaaugggcacuacgacagaucgccgacuuugcccuaaaacuacacaaaacgcaccuggccucuuuucuuucagccuucgcacgccaagaacucuaccucaugggcagccucguccacuccaugcugguacauacgacggagagacgcgaaaucuucaucguagaaacgggccucuguucauuggccgagcuaucacacuuuacgcaguuguuagcucauccacaccacgaauaccucagcgaccuguacacacccuguuccaguagcgggcgacgcgaucacucgcucgaacgccucacgcgucucuuccccgaugccaccguccccgcuaccguucccgccgcccucuccauccuaucuaccaugcaaccaagcacgcuggaaaccuuccccgaccuguuuugcuugccgcucggcgaauccuucuccgcgcugaccgucuccgaacacgucaguuauaucguaacaaaccaguaccugaucaaagguaucuccuacccugucuccaccaccgucguaggccagagccucaucaucacccagacggacagucaaacuaaaugcgaacugacgcgcaacaugcauaccacacacagcaucacaguggcgcucaacauuucgcuagaaaacugcgccuuuugccaaagcgcccugcuagaauacgacgacacgcaaggcgucaucaacaucauguacaugcacgacucggacgacguccuuuucgcccuggaucccuacaacgaagugguggucucaucuccgcgaacucacuaccucaugcuuuugaagaacgguacgguacuagaaguaacugacgucgucguggacgccaccgacagucgucuccucaugauguccgucuacgcgcuaucggccaucaucggcaucuaucugcucuaccgcaugcucaagacaugcugauaauaggcuggagccucgguggccaugcuucuugccccuugggccuccccccagccccuccuccccuuccugcacccguacccccguggucuuugaauaaagucugagugggcggc

SEQ ID NO: 6 - RNA, 1000 nt, Artificial Sequence
  Synthetic
gggaaauaagagagaaaagaagaguaagaagaaauauaagagccaccaugugccgccgcccggauugcggcuucucuuucucaccuggaccggugauacugcugugguguugccuucugcugcccauuguuuccucagccgccgucagcgucgcuccuaccgccgccgagaaaguccccgcggagugccccgaacuaacgcgccgaugcuuguugggugagguguuugagggugacaaguaugaaaguuggcugcgcccguuggugaauguuaccgggcgcgauggcccgcuaucgcaacuuauccguuaccgucccguuacgccggaggccgccaacuccgugcuguuggacgaggcuuuccuggacacucuggcccugcuguacaacaauccggaucaauugcgggcccugcugacgcuguugagcucggacacagcgccgcgcuggaugacggugaugcgcggcuacagcgagugcggcgauggcucgccggccguguacacgugcguggacgaccugugccgcggcuacgaccucacgcgacugucauacgggcgcagcaucuucacggaacacguguuaggcuucgagcuggugccaccgucucucuuuaacguggugguggccauacgcaacgaagccacgcguaccaaccgcgccgugcgucugcccgugagcaccgcugccgcgcccgagggcaucacgcucuuuuacggccuguacaacgcagugaaggaauucugccugcgucaccagcuggacccgccgcugcuacgccaccuagauaaauacuacgccggacugccgcccgagcugaagcagacgcgcgucaaccugccggcucacucgcgcuauggcccucaagcaguggaugcucgcugauaauaggcuggagccucgguggccaugcuucuugccccuugggccuccccccagccccuccuccccuuccugcacccguacccccguggucuuugaauaaagucugagugggcggc

SEQ ID NO: 7 - RNA, 2721 nt, Artificial Sequence
  Synthetic
auggaauccaggaucuggugccugguagucugcguuaacuuguguaucgucugucugggugcugcgguuuccucaucuucuacucguggaacuucugcuacucacagucaccauuccucucauacgacgucugcugcucacucucgauccgguucagucucucaacgcguaacuucuucccaaacggucagccaugguguuaacgagaccaucuacaacacuacccucaaguacggagauguggugggggucaauaccaccaaguaccccuaucgcguguguucuauggcccaggguacggaucuuauucgcuuugaacguaauaucgucugcaccucgaugaagcccaucaaugaagaccuggacgagggcaucaugguggucuacaaacgcaacaucgucgcgcacaccuuuaagguacgagucuaccagaagguuuugacguuucgucguagcuacgcuuacauccacaccacuuaucugcugggcagcaacacggaauacguggcgccuccuaugugggagauucaucauaucaacagccacagucagugcuacaguuccuacagccgcguuauagcaggcacgguuuucguggcuuaucauagggacagcuaugaaaacaaaaccaugcaauuaaugcccgacgauuauuccaacacccacaguacccguuacgugacggucaaggaucaauggcacagccgcggcagcaccuggcucuaucgugagaccuguaaucugaauuguauggugaccaucacuacugcgcgcuccaaauauccuuaucauuuuuucgccacuuccacgggugacgugguugacauuucuccuuucuacaacggaaccaaucgcaaugccagcuacuuuggagaaaacgccgacaaguuuuucauuuuuccgaacuacacuaucgucuccgacuuuggaagaccgaauucugcguuagagacccacagguugguggcuuuucuugaacgugcggacucggugaucuccugggauauacaggacgaaaagaaugucacuugucaacucacuuucugggaagccucggaacgcaccauucguuccgaagccgaggacucguaucacuuuucuucugccaaaaugaccgccacuuucuuaucuaagaagcaagaggugaacauguccgacucugcgcuggacugcguacgugaugaggcuauaaauaaguuacagcagauuuucaauacuucauacaaucaaacauaugaaaaauauggaaacguguccgucuuugaaaccacuggugguuugguaguguucuggcaagguaucaagcaaaaaucucugguggaacucgaacguuuggccaaccgcuccagucugaaucuuacucauaauagaaccaaaagaaguacagauggcaacaaugcaacucauuuauccaacauggaaucggugcacaaucuggucuacgcccagcugcaguucaccuaugacacguugcgcgguuacaucaaccgggcgcuggcgcaaaucgcagaagccugguguguggaucaacggcgcacccuagaggucuucaaggaacucagcaagaucaacccgucagccauucucucggccauuuacaacaaaccgauugccgcgcguuucaugggugaugucuugggccuggccagcugcgugaccaucaaccaaaccagcgucaaggugcugcgugauaugaacgugaaggagucgccaggacgcugcuacucacgacccguggucaucuuuaauuucgccaacagcucguacgugcaguacggucaacugggcgaggacaacgaaauccuguugggcaaccaccgcacugaggaaugucagcuucccagccucaagaucuucaucgccgggaacucggccuacgaguacguggacuaccucuucaaacgcaugauugaccucagcaguaucuccaccgucgacagcaugaucgcccuggauaucgacccgcuggaaaauaccgacuucaggguacuggaacuuuacucgcagaaagagcugcguuccagcaacguuuuugaccucgaagagaucaugcgcgaauucaacucguacaagcagcggguaaaguacguggaggacaagguagucgacccgcuaccgcccuaccucaagggucuggacgaccucaugagcggccugggcgccgcgggaaaggccguuggcguagccauuggggccguggguggcgcgguggccuccguggucgaaggcguugccaccuuccucaaaaaccccuucggagcguucaccaucauccucguggccauagcuguagucauuaucacuuauuugaucuauacucgacagcggcguuugugcacgcagccgcugcagaaccucuuucccuaucugguguccgccgacgggaccaccgugacgucgggcagcaccaaagacacgucguuacaggcuccgccuuccuacgaggaaaguguuuauaauucuggucgcaaaggaccgggaccaccgucgucugaugcauccacggcggcuccgccuuacaccaacgagcaggcuuaccagaugcuucuggcccuggcccgucuggacgcagagcagcgagcgcagcagaacgguacagauucuuuggacggacggacuggcacgcaggacaagggacagaagcccaaccuacuagaccgacugcgacaucgcaaaaacggcuaccgacacuugaaagacucugacgaagaagagaacguc

SEQ ID NO: 8 - RNA, 513 nt, Artificial Sequence
  Synthetic
augagucccaaagaucugacgccguucuugacggcguuguggcugcuauugggucacagccgcgugccgcgggugcgcgcagaagaauguugcgaauucauaaacgucaaccacccgccggaacgcuguuacgauuucaaaaugugcaaucgcuucaccgucgcgcugcgguguccggacggcgaagucugcuacagucccgagaaaacggcugagauucgcgggaucgucaccaccaugacccauucauugacacgccaggucguacacaacaaacugacgagcugcaacuacaauccguuauaccucgaagcugacgggcgaauacgcugcggcaaaguaaacgacaaggcgcaguaccugcugggcgccgcuggcagcguucccuaucgauggaucaaucuggaauacgacaagauaacccggaucgugggccuggaucaguaccuggagagcguuaagaaacacaaacggcuggaugugugccgcgcuaaaaugggcuauaugcugcag

SEQ ID NO: 9 - RNA, 642 nt, Artificial Sequence
  Synthetic
augcugcggcuucugcuucgucaccacuuucacugccugcuucugugcgcgguuugggcaacgcccugucuggcgucuccguggucgacgcuaacagcaaaccagaauccguccccgccauggucuaaacugacguauuccaaaccgcaugacgcggcgacguuuuacuguccuuuucucuaucccucgcccccacgaucccccuugcaauucucgggguuccagcggguaucaacgggucccgagugucgcaacgagacccuguaucugcuguacaaccgggaaggccagaccuugguggagagaagcuccaccugggugaaaaaggugaucugguaccugagcggucggaaccaaaccauccuccaacggaugccccgaacggcuucgaaaccgagcgacggaaacgugcagaucagcguggaagacgccaagauuuuuggagcgcacauggugcccaagcagaccaagcugcuacgcuucgucgucaacgauggcacacguuaucagaugugugugaugaagcuggagagcugggcucacgucuuccgggacuacagcgugucuuuucaggugcgauugacguucaccgaggccaauaaccagacuuacaccuucugcacccaucccaaucucaucguu

SEQ ID NO: 10 - RNA, 387 nt, Artificial Sequence
  Synthetic
augcggcugugucggguguggcugucuguuugucugugcgccguggugcugggucagugccagcgggaaaccgcggaaaagaacgauuauuaccgaguaccgcauuacugggacgcgugcucucgcgcgcugcccgaccaaacccguuacaaguauguggaacagcucguggaccucacguugaacuaccacuacgaugcgagccacggcuuggacaacuuugacgugcucaagagaaucaacgugaccgaggugucguugcucaucagcgacuuuagacgucagaaccgucgcggcggcaccaacaaaaggaccacguucaacgccgccgguucgcuggcgccacacgcccggagccucgaguucagcgugcggcucuuugccaac

SEQ ID NO: 11 - RNA, 2226 nt, Artificial Sequence
  Synthetic
augcggccaggccuccccuccuaccucaucauccucgccgucugucucuucagccaccuacuuucgucacgauauggcgcagaagccguauccgaaccgcuggacaaagcguuucaccuacugcucaacaccuacgggagacccauccgcuuccugcgugaaaauaccacccaguguaccuacaacagcagccuccguaacagcacggucgucagggaaaacgccaucaguuucaacuucuuccaaagcuauaaucaauacuauguauuccauaugccucgaugucucuuugcggguccucuggcggagcaguuucugaaccagguagaucugaccgaaacccuggaaagauaccaacagagacuuaacacuuacgcgcugguauccaaagaccuggccagcuaccgaucuuucucgcagcagcuaaaggcacaagacagccuaggugaacagcccaccacugugccaccgcccauugaccugucaauaccucacguuuggaugccaccgcaaaccacuccacacggcuggacagaaucacauaccaccucaggacuacaccgaccacacuuuaaccagaccuguauccucuuugauggacacgaucuacuauucagcaccgucacaccuuguuugcaccaaggcuuuuaccucaucgacgaacuacguuacguuaaaauaacacugaccgaggacuucuucguaguuacgguguccauagacgacgacacacccaugcugcuuaucuucggccaucuuccacgcguacuuuucaaagcgcccuaucaacgcgacaacuuuauacuacgacaaacugagaaacacgagcuccuggugcuaguuaagaaagaucaacugaaccgucacucuuaucucaaagacccggacuuucuugacgccgcacuugacuucaacuaccuagaccucagcgcacuacuacguaacagcuuucaccguuacgccguggauguacucaagagcggucgaugucagaugcuggaccgccgcacgguagaaauggccuucgccuacgcauuagcacuguucgcagcagcccgacaagaagaggccggcgcccaagucuccgucccacgggcccuagaccgccaggccgcacucuuacaaauacaagaauuuaugaucaccugccucucacaaacaccaccacgcaccacguugcugcuguaucccacggccguggaccuggccaaacgagcccuuuggacaccgaaucagaucaccgacaucaccagccucguacgccuggucuacauacucucuaaacagaaucagcaacaucucaucccccaaugggcacuacgacagaucgccgacuuugcccuaaaacuacacaaaacgcaccuggccucuuuucuuucagccuucgcacgccaagaacucuaccucaugggcagccucguccacuccaugcugguacauacgacggagagacgcgaaaucuucaucguagaaacgggccucuguucauuggccgagcuaucacacuuuacgcaguuguuagcucauccacaccacgaauaccucagcgaccuguacacacccuguuccaguagcgggcgacgcgaucacucgcucgaacgccucacgcgucucuuccccgaugccaccguccccgcuaccguucccgccgcccucuccauccuaucuaccaugcaaccaagcacgcuggaaaccuuccccgaccuguuuugcuugccgcucggcgaauccuucuccgcgcugaccgucuccgaacacgucaguuauaucguaacaaaccaguaccugaucaaagguaucuccuacccugucuccaccaccgucguaggccagagccucaucaucacccagacggacagucaaacuaaaugcgaacugacgcgcaacaugcauaccacacacagcaucacaguggcgcucaacauuucgcuagaaaacugcgccuuuugccaaagcgcccugcuagaauacgacgacacgcaaggcgucaucaacaucauguacaugcacgacucggacgacguccuuuucgcccuggaucccuacaacgaagugguggucucaucuccgcgaacucacuaccucaugcuuuugaagaacgguacgguacuagaaguaacugacgucgucguggacgccaccgacagucgucuccucaugauguccgucuacgcgcuaucggccaucaucggcaucuaucugcucuaccgcaugcucaagacaugc

SEQ ID NO: 12 - RNA, 834 nt, Artificial Sequence
  Synthetic
augugccgccgcccggauugcggcuucucuuucucaccuggaccggugauacugcugugguguugccuucugcugcccauuguuuccucagccgccgucagcgucgcuccuaccgccgccgagaaaguccccgcggagugccccgaacuaacgcgccgaugcuuguugggugagguguuugagggugacaaguaugaaaguuggcugcgcccguuggugaauguuaccgggcgcgauggcccgcuaucgcaacuuauccguuaccgucccguuacgccggaggccgccaacuccgugcuguuggacgaggcuuuccuggacacucuggcccugcuguacaacaauccggaucaauugcgggcccugcugacgcuguugagcucggacacagcgccgcgcuggaugacggugaugcgcggcuacagcgagugcggcgauggcucgccggccguguacacgugcguggacgaccugugccgcggcuacgaccucacgcgacugucauacgggcgcagcaucuucacggaacacguguuaggcuucgagcuggugccaccgucucucuuuaacguggugguggccauacgcaacgaagccacgcguaccaaccgcgccgugcgucugcccgugagcaccgcugccgcgcccgagggcaucacgcucuuuuacggccuguacaacgcagugaaggaauucugccugcgucaccagcuggacccgccgcugcuacgccaccuagauaaauacuacgccggacugccgcccgagcugaagcagacgcgcgucaaccugccggcucacucgcgcuauggcccucaagcaguggaugcucgc

SEQ ID NO: 13 - RNA, 47 nt, Artificial Sequence
  Synthetic
gggaaauaagagagaaaagaagaguaagaagaaauauaagagccacc

SEQ ID NO: 14 - RNA, 119 nt, Artificial Sequence
  Synthetic
ugauaauaggcuggagccucgguggccaugcuucuugccccuugggccuccccccagccccuccuccccuuccugcacccguacccccguggucuuugaauaaagucugagugggcggc

SEQ ID NO: 15 - protein, 907 aa, Artificial Sequence
  Synthetic
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 16 - protein, 171 aa, Artificial Sequence
  Synthetic
MSPKDLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 17 - protein, 214 aa, Artificial Sequence
  Synthetic
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 18 - protein, 129 aa, Artificial Sequence
  Synthetic
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 19 - protein, 742 aa, Artificial Sequence
  Synthetic
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 20 - protein, 278 aa, Artificial Sequence
  Synthetic
MCRRPDCGFSFSPGPVILLWCCLLLPIVSSAAVSVAPTAAEKVPAECPELTRRCLLGEVFEGDKYESWLRPLVNVTGRDGPLSQLIRYRPVTPEAANSVLLDEAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 21 - RNA, 1837 nt, Artificial Sequence
  Synthetic
gggaaauaagagagaaaagaagaguaagaagaaauauaagagccaccauggagucgcgcggucgccguugucccgaaaugauauccguacugggucccauuucggggcacgugcugaaagccguguuuagucgcggcgauacgccggugcugccgcacgagacgagacuccugcagacggguauccacguacgcgugagccagcccucgcugauccuggugucgcaguacacgcccgacucgacgccaugccaccgcggcgacaaucagcugcaggugcagcacacguacuuuacgggcagcgagguggagaacgugucggucaacgugcacaaccccacgggccgaagcaucugccccagccaagagcccaugucgaucuauguguacgcgcugccgcucaagaugcugaacauccccagcaucaacgugcaccacuacccgucggcggccgagcgcaaacaccgacaccugcccguagccgacgcuguuauucacgcgucgggcaagcagauguggcaggcgcgucucacggucucgggacuggccuggacgcgucagcagaaccaguggaaagagcccgacgucuacuacacgucagcguucguguuucccaccaaggacguggcacugcggcacguggugugcgcgcacgagcugguuugcuccauggagaacacgcgcgcaaccaagaugcaggugauaggugaccaguacgucaagguguaccuggaguccuucugcgaggacgugcccuccggcaagcucuuuaugcacgucacgcugggcucugacguggaagaggaccuaacgaugacccgcaacccgcaacccuucaugcgcccccacgagcgcaacggcuuuacgguguugugucccaaaaauaugauaaucaaaccgggcaagaucucgcacaucaugcuggauguggcuuuuaccucacacgagcauuuugggcugcugugucccaagagcaucccgggccugagcaucucagguaaccuguugaugaacgggcagcaaaucuuccuggagguacaagcgauacgcgagaccguggaacugcgucaguacgaucccguggcugcgcucuucuuuuucgauaucgacuuguugcugcagcgcgggccucaguacagcgagcaccccaccuucaccagccaguaucgcauccagggcaagcuugaguaccgacacaccugggaccggcacgacgagggugccgcccagggcgacgacgacgucuggaccagcggaucggacuccgacgaagaacucguaaccaccgagcguaagacgccccgcgucaccggcggcggagccauggcgagcgccuccacuuccgcgggcucagcauccucggcgacggcgugcacggcgggcguuaugacacgcggccgccuuaaggccgaguccaccgucgcgcccgaagaggacaccgacgaggauuccgacaacgaaauccacaauccggccguguucaccuggccgcccuggcaggccggcauccuggcccgcaaccuggugcccaugguggcuacgguucagggucagaaucugaaguaccaggaguucuucugggacgccaacgacaucuaccgcaucuucgccgaauuggaaggcguauggcagcccgcugcgcaacccaaacgucgccgccaccggcaagacgccuugcccgggccaugcaucgccucgacgcccaaaaagcaccgagguugauaauaggcuggagccucgguggccaugcuucuugccccuugggccuccccccagccccuccuccccuuccugcacccguacccccguggucuuugaauaaagucugagugggcggc

SEQ ID NO: 22 - protein, 20 aa, Artificial Sequence
  Synthetic
METPAQLLFLLLLWLPDTTG

SEQ ID NO: 23 - RNA, 1671 nt, Artificial Sequence
  Synthetic
auggagucgcgcggucgccguugucccgaaaugauauccguacugggucccauuucggggcacgugcugaaagccguguuuagucgcggcgauacgccggugcugccgcacgagacgagacuccugcagacggguauccacguacgcgugagccagcccucgcugauccuggugucgcaguacacgcccgacucgacgccaugccaccgcggcgacaaucagcugcaggugcagcacacguacuuuacgggcagcgagguggagaacgugucggucaacgugcacaaccccacgggccgaagcaucugccccagccaagagcccaugucgaucuauguguacgcgcugccgcucaagaugcugaacauccccagcaucaacgugcaccacuacccgucggcggccgagcgcaaacaccgacaccugcccguagccgacgcuguuauucacgcgucgggcaagcagauguggcaggcgcgucucacggucucgggacuggccuggacgcgucagcagaaccaguggaaagagcccgacgucuacuacacgucagcguucguguuucccaccaaggacguggcacugcggcacguggugugcgcgcacgagcugguuugcuccauggagaacacgcgcgcaaccaagaugcaggugauaggugaccaguacgucaagguguaccuggaguccuucugcgaggacgugcccuccggcaagcucuuuaugcacgucacgcugggcucugacguggaagaggaccuaacgaugacccgcaacccgcaacccuucaugcgcccccacgagcgcaacggcuuuacgguguugugucccaaaaauaugauaaucaaaccgggcaagaucucgcacaucaugcuggauguggcuuuuaccucacacgagcauuuugggcugcugugucccaagagcaucccgggccugagcaucucagguaaccuguugaugaacgggcagcaaaucuuccuggagguacaagcgauacgcgagaccguggaacugcgucaguacgaucccguggcugcgcucuucuuuuucgauaucgacuuguugcugcagcgcgggccucaguacagcgagcaccccaccuucaccagccaguaucgcauccagggcaagcuugaguaccgacacaccugggaccggcacgacgagggugccgcccagggcgacgacgacgucuggaccagcggaucggacuccgacgaagaacucguaaccaccgagcguaagacgccccgcgucaccggcggcggagccauggcgagcgccuccacuuccgcgggcucagcauccucggcgacggcgugcacggcgggcguuaugacacgcggccgccuuaaggccgaguccaccgucgcgcccgaagaggacaccgacgaggauuccgacaacgaaauccacaauccggccguguucaccuggccgcccuggcaggccggcauccuggcccgcaaccuggugcccaugguggcuacgguucagggucagaaucugaaguaccaggaguucuucugggacgccaacgacaucuaccgcaucuucgccgaauuggaaggcguauggcagcccgcugcgcaacccaaacgucgccgccaccggcaagacgccuugcccgggccaugcaucgccucgacgcccaaaaagcaccgaggu

SEQ ID NO: 24 - protein, 557 aa, Artificial Sequence
  Synthetic
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 25 - protein, 30 aa, Artificial Sequence
  Synthetic
MDSKGSSQKGSRLLLLLVVSNLLLPQGVVG

SEQ ID NO: 26 - protein, 18 aa, Artificial Sequence
  Synthetic
MDWTWILFLVAAATRVHS

SEQ ID NO: 27 - protein, 24 aa, Artificial Sequence
  Synthetic
MLGSNSGQRVVFTILLLLVAPAYS

SEQ ID NO: 28 - protein, 17 aa, Artificial Sequence
  Synthetic
MKCLLYLAFLFIGVNCA

SEQ ID NO: 29 - protein, 15 aa, Artificial Sequence
  Synthetic
MWLVSLAIVTACAGA

SEQ ID NO: 30 - RNA, 9 nt, Artificial Sequence
  Synthetic
ccrccaugg

SEQ ID NO: 31 - RNA, 11 nt, Artificial Sequence
  Synthetic
gggauccuacc

SEQ ID NO: 32 - RNA, 9 nt, Artificial Sequence
  Synthetic
  misc_feature (8)..(9): may be absent
uuauuuaww